(19) United States
(12) Patent Application Publication
(10) Pub. No.: US 2012/0114646 A1
(43) Pub. Date: May 10, 2012

(54) LYOPHILIZED FORMULATIONS FOR SMALL MODULAR IMMUNOPHARMACEUTICALS

(75) Inventors: Serguei Tchessalov, Andover, MA (US); Angela Kantor, Pepperell, MA (US); Li Li, Sudbury, MA (US); Nicholas Luksha, Andover, MA (US); Nicholas Warne, Andover, MA (US)

(73) Assignee: WYETH LLC, Madison, NJ (US)

(21) Appl. No.: 13/378,751

(22) PCT Filed: Jun. 18, 2010
(86) PCT No.: PCT/US10/39227

§ 371 Date: Jan. 18, 2012

Related U.S. Application Data
(60) Provisional application No. 61/218,386, filed on Jun. 18, 2009.
(60) Provisional application No. 61/218,388, filed on Jun. 18, 2009.

(51) Int. Cl. A61K 39/395; A61P 37/00

(52) U.S. Cl. 424/134.1

(57) ABSTRACT

The present invention provides, among other things, stable formulations for small modular immunopharmaceutical (SMIP™) proteins. In some embodiments, the present invention provides a formulation containing a lyophilized mixture of a small modular immunopharmaceutical protein, wherein less than 7% of the lyophilized small modular immunopharmaceutical protein exists in aggregated form. Formulations according to the invention may contain buffering agents, stabilizers, bulking agents, surfactants and/or other excipients. The present invention also provides formulations for lyophilization, reconstitution and methods of use thereof.

### CROSS REFERENCE TO RELATED APPLICATIONS

This application claims priority to U.S. Provisional Patent Application Ser. Nos. 61/218,388 and 61/218,386 both filed on Jun. 18, 2009; the entirety of each of which is hereby incorporated by reference.

### BACKGROUND OF THE INVENTION

In the past ten years, advances in biotechnology have made it possible to produce a variety of proteins for pharmaceutical applications. Because proteins are larger and more complex than traditional organic and inorganic drugs (i.e., possessing multiple functional groups in addition to complex three-dimensional structures), the formulation, packaging and preservation of such proteins poses special problems. A liquid formulation is generally desirable due to clinical convenience, patient convenience and manufacturing ease. For many proteins, however, a liquid formulation is not feasible. The complexity of the protein leads to protein degradation from the stresses encountered during manufacturing, packaging and shipping. Certain small modular immunopharmaceuticals belong to this category.

As a result, when a liquid formulation is not an option, lyophilization provides reasonable assurance of producing a stable dosage form under acceptable shipping and storage conditions. Lyophilization generally includes three main stages: freezing, primary drying and secondary drying. Freezing converts water to ice or some amorphous formulation components to the crystalline form. Primary drying is the process step when ice is removed from the frozen product by direct sublimation at low pressure and temperature. Secondary drying is the process step when bounded water is removed from the product matrix utilizing the diffusion of residual water to the evaporation surface. Therefore, appropriate choice of excipients and other formulation components is needed to prevent proteins from freezing and dehydration stresses and to enhance protein stability during freeze-drying and/or to improve stability of lyophilized product during storage.

### SUMMARY OF THE INVENTION

The present invention encompasses the discovery that stable lyophilized formulations can be prepared using combinations of buffering agents, stabilizers, bulking agents and/or surfactants for small modular immunopharmaceutical proteins. Thus, the present invention provides, among other things, stable formulations containing a lyophilized small modular immunopharmaceutical protein.

In one aspect, the present invention provides formulations containing a lyophilized mixture of a small modular immunopharmaceutical protein. In some embodiments, less than 10%, 9%, 8%, 7%, 6%, 5%, 4%, 3%, 2%, 1%, or 0.5% of the lyophilized small modular immunopharmaceutical protein exists in aggregated form. In certain embodiments, less than 10%, 9%, 8%, 7%, 6%, 5%, 4%, 3%, 2%, 1%, or 0.5% of the lyophilized small modular immunopharmaceutical protein exists in aggregated form upon storage at 2-8° C. for at least 1 month, 3 months, 6 months, 1 year or 2 years. In certain embodiments, less than 10%, 9%, 8%, 7%, 6%, 5%, 4%, 3%, 2%, 1%, or 0.5% of the lyophilized small modular immunopharmaceutical protein exists in aggregated form upon storage at 25° C. or room temperature for at least 1 month, 3 months, 6 months, 1 year or 2 years. In certain embodiments, less than 10%, 9%, 8%, 7%, 6%, 5%, 4%, 3%, 2%, 1%, or 0.5% of the lyophilized small modular immunopharmaceutical protein exists in aggregated form upon storage at 40° C. for at least 2 weeks, 1 month, 3 months, or 6 months.

In some embodiments, a formulation according to the present invention contains a bulking agent, a stabilizing agent and/or a buffering agent. In some embodiments, a bulking agent suitable for the invention is selected from the group consisting of sucrose, mannitol, glycine, sodium chloride, dextran, trehalose, and combinations thereof. In some embodiments, a buffering agent suitable for the invention is selected from the group consisting of histidine, sodium acetate, citrate, phosphate, succinate, Tris, and combinations thereof. In some embodiments, a stabilizing agent suitable for the invention is selected from the group consisting of sucrose, sorbitol, mannitol, glycine, trehalose, and combinations thereof.

In some embodiments, a formulation of the invention further includes an isotonicity agent. In some embodiments, an isotonicity agent suitable for the inventions is selected from the group consisting of glycine, sorbitol, sucrose, mannitol, sodium chloride, dextrose, arginine, and combinations thereof.

In some embodiments, a formulation of the invention includes a non-reducing sugar. In some embodiments, the non-reducing sugar is sucrose or trehalose. In some embodiments, the mass ratio of the non-reducing sugar to the small modular immunopharmaceutical protein is about 0.1:1, 0.2:1, 0.25:1, 0.4:1, 0.5:1, 1:1, 2:1, 2.6:1, 3:1, 4:1, or 5:1.

In some embodiments, a formulation of the invention further includes a surfactant. In some embodiments, a surfactant suitable for the invention is selected from the group consisting of Polysorbate 20, Polysorbate 80, poloxamers, Triton, and combinations thereof.

In certain embodiments, the present invention provides a formulation that includes a lyophilized mixture of a small modular immunopharmaceutical protein, sucrose, histidine and Polysorbate 80. In certain embodiments, the present invention provides a formulation that includes a lyophilized mixture of a small modular immunopharmaceutical protein, sucrose, mannitol, and a buffering agent selected from histidine and/or sodium acetate

In some embodiments, a mass ratio of mannitol to sucrose in a formulation of the invention is about 0.1:1, 0.5:1, 1:1, 2:1, 3:1, 4:1, 5:1, or 10:1.

In some embodiments, the present invention provides a lyophilized mixture of a small modular immunopharmaceutical protein, sucrose, glycine and sodium acetate.

In some embodiments, inventive formulations of the invention contain a small modular immunopharmaceutical protein that includes a binding domain that specifically targets CD20. In some embodiments, the small modular immunopharmaceutical protein has an amino acid sequence having at least 80% identity to any one of SEQ ID NOs: 1-59 and 67-76.

In various embodiments, the lyophilized small modular immunopharmaceutical protein according to the invention is stable during storage, for example, at 2-8° C. (e.g., 5° C.) or room temperature (e.g., 25° C.).

A formulation comprising a lyophilized mixture of a small modular immuno-pharmaceutical protein, sucrose, histidine, and Polysorbate 80.

In another aspect, the present invention provides reconstituted formulations of lyophilized formulations as described herein. In some embodiments, a reconstituted formulation includes a diluent, and the small modular immunopharmaceutical protein at a concentration in the range of about 25 mg/ml to about 400 mg/ml (e.g., about 25 mg/ml to about 200 mg/ml; about 50 mg/ml to about 200 mg/ml; about 25 mg/ml to about 150 mg/ml; about 100 mg/ml to about 250 mg/ml, about 100 mg/ml to about 300 mg/ml, about 200 mg/ml to about 400 mg/ml, about 300 mg/ml to about 400 mg/ml). In some embodiments, a reconstituted formulation includes a diluent, and a small modular immunopharmaceutical protein at a concentration of approximately 25 mg/ml, 50 mg/ml, 75 mg/ml, 100 mg/ml, 125 mg/ml, 150 mg/ml, 175 mg/ml, 200 mg/ml, 250 mg/ml, 300 mg/ml, 350 mg/ml, or 400 mg/ml.

In some embodiments, the reconstituted formulation is for intravenous, subcutaneous, or intramuscular administration.

The present invention also provides methods for treating a patient by administering a reconstituted formulation of the invention and kits or other articles of manufacture, including a container which holds a lyophilized formulation of the invention.

In yet another aspect, the present invention provides for a formulation for lyophilization comprising a small modular immunopharmaceutical protein, a non-reducing sugar, and a buffering agent. In some embodiments, the buffering agent is selected from sodium acetate or histidine. In some embodiments, the buffering agent is at a concentration of approximately 5 mM, 10 mM, 15 mM, 20 mM, 25 mM, or 30 mM. In some embodiments, histidine is at a concentration of approximately 5 mM, 10 mM, 15 mM, 20 mM, 25 mM, or 30 mM.

In some embodiments, the formulation further includes mannitol. In some embodiments, the formulation, further includes methionine. In some embodiments, the methionine is at a concentration of approximately 10 mM. In some embodiments, the non-reducing sugar is sucrose. In some embodiments, the sucrose is at a concentration ranging between approximately 0.5% and 15% (e.g., approximately 1% and 10%, 5% and 15%, 5% and 10%). In some embodiments, the sucrose is at a concentration of approximately 5%. In some embodiments, a suitable formulation contains sucrose at a concentration of approximately 10% and histidine at a concentration of approximately 20 mM. In some embodiments, the mass ratio of the non-reducing sugar to the small modular immunopharmaceutical protein is about 0.1:1, 0.2:1, 0.25:1, 0.4:1, 0.5:1, 1:1, 2:1, 2.6:1, 3:1, 4:1, or 5:1.

In some embodiments, a suitable formulation for lyophilization further includes an isotonicity agent. In some embodiments, the isotonicity agent is glycine, sorbitol, sucrose, mannitol, sodium chloride, dextrose, and/or arginine. In some embodiments, a suitable formulation for lyophilization further includes a surfactant. In some embodiments, a suitable surfactant is Polysorbate 20, Polysorbate 80, poloxamers, and/or Triton.

In various embodiments, formulations for lyophilization according to the invention contain the small modular immunopharmaceutical protein at a concentration in the range of about 25 mg/ml to about 400 mg/ml (e.g., about 25 mg/ml to about 200 mg/ml; about 50 mg/ml to about 200 mg/ml; about 25 mg/ml to about 150 mg/ml; about 100 mg/ml to about 250 mg/ml, about 100 mg/ml to about 300 mg/ml, about 200 mg/ml to about 400 mg/ml, about 300 mg/ml to about 400 mg/ml). In some embodiments, formulations for lyophilization according to the invention contain a small modular immunopharmaceutical protein at a concentration of approximately 25 mg/ml, 50 mg/ml, 75 mg/ml, 100 mg/ml, 125 mg/ml, 150 mg/ml, 175 mg/ml, 200 mg/ml, 250 mg/ml, 300 mg/ml, 350 mg/ml, or 400 mg/ml.

In some embodiments, the present invention provides a formulation for lyophilization containing a small modular immunopharmaceutical protein, sucrose at a concentration ranging between approximately 5% and 10%, histidine at a concentration ranging between approximately 10 mM and 20 mM, and Polysorbate 80 at a concentration ranging between approximately 0.001% and 0.1%.

In some embodiments, the present invention provides a formulation for lyophilization containing a small modular immunopharmaceutical protein at a concentration of approximately 25 mg/ml, sucrose at a concentration of approximately 6.5%, glycine at a concentration of approximately 50 mM, and sodium acetate at a concentration of approximately 20 mM.

In some embodiments, the present invention provides a formulation for lyophilization containing a small modular immunopharmaceutical protein at a concentration ranging between approximately 50 mg/ml and 100 mg/ml, histidine at a concentration of approximately 20 mM, mannitol at a concentration of approximately 4%, and sucrose at a concentration of approximately 1%.

In some embodiments, the present invention provides a formulation for lyophilization containing a small modular immunopharmaceutical protein at a concentration of approximately 100 mg/ml, sucrose at a concentration of approximately 10%, histidine at a concentration of approximately 20 mM, Polysorbate-80 at a concentration of approximately 0.01%.

In some embodiments, the present invention provides a formulation for lyophilization containing a small modular immunopharmaceutical protein at a concentration of approximately 100 mg/ml, sucrose at a concentration of approximately 5%, glycine at a concentration of approximately 1%, histidine at a concentration of approximately 20 mM, Polysorbate-80 at a concentration of approximately 0.01%.

In some embodiments, the present invention provides a formulation for lyophilization containing a small modular immunopharmaceutical protein at a concentration of approximately 100 mg/ml, sucrose at a concentration of approximately 5%, sorbitol at a concentration of approximately 2.4%, histidine at a concentration of approximately 20 mM, Polysorbate-80 at a concentration of approximately 0.01%.

In some embodiments, the present invention provides a formulation for lyophilization containing a small modular immunopharmaceutical protein at a concentration of approximately 200 mg/ml, sucrose at a concentration ranging between 5% and 10%, histidine at a concentration of approximately 20 mM, Polysorbate-80 at a concentration of approximately 0.01%.

In some embodiments, the present invention provides a formulation for lyophilization containing a small modular immunopharmaceutical protein, sucrose at a concentration of approximately 5%, histidine at a concentration of approximately 10 mM, methionine at a concentration of approximately 10 mM, and polysorbate 80 at a concentration of approximately 0.01%.

In some embodiments, the formulation has a pH ranging from approximately 5.0 to approximately 7.0.

In some embodiments, wherein the formulation has a pH of 6.0.

In various embodiments, formulations for lyophilization according to the invention include a small modular immunopharmaceutical protein that contains a binding domain that specifically targets CD20. In certain embodiments, the small modular immunopharmaceutical protein has an amino acid sequence having at least 80% identity to any one of SEQ ID NOs: 1-59 and 67-76.

In still another aspect, the present invention provides a method of storing a small modular immunopharmaceutical protein including lyophilizing a formulation containing a small modular immunopharmaceutical protein and storing the lyophilized formulation at a temperature at or lower than room temperature.

In some embodiments, inventive methods of the invention are utilized to store a small modular immunopharmaceutical protein that contains a binding domain that specifically targets CD20. In certain embodiments, the small modular immunopharmaceutical protein has an amino acid sequence having at least 80% identity to any one of SEQ ID NOs: 1-59 and 67-76.

In some embodiments, a method of the invention includes storing the lyophilized formulation at a temperature of about 2-8° C. (e.g., 5° C.). In some embodiments, a method of the invention includes storing the lyophilized formulation at about room temperature.

The present invention also provides lyophilized and/or stored small modular immunopharmaceutical proteins using methods and/or formulations described herein.

As used in this application, the terms “about” and “approximately” are used as equivalents. Any numerals used in this application with or without about/approximately are meant to cover any normal fluctuations appreciated by one of ordinary skill in the relevant art. For example, normal fluctuations of a value of interest may include a range of values that fall within 25%, 20%, 19%, 18%, 17%, 16%, 15%, 14%, 13%, 12%, 11%, 10%, 9%, 8%, 7%, 6%, 5%, 4%, 3%, 2%, 1%, or less in either direction (greater than or less than) of the stated reference value unless otherwise stated or otherwise evident from the context (except where such number would exceed 100% of a possible value).

Other features, objects, and advantages of the present invention are apparent in the detailed description that follows. It should be understood, however, that the detailed description, while indicating embodiments of the present invention, is given by way of illustration only, not limitation. Various changes and modifications within the scope of the invention will become apparent to those skilled in the art from the detailed description.

### BRIEF DESCRIPTION OF THE DRAWINGS

The drawings are for illustration purposes only, not for limitation.

FIG. 1 illustrates the structure of an exemplary small modular immunopharmaceutical protein (SMIP™).

FIG. 2 illustrates exemplary lyophilization cycle for the protein at 25 mg/ml in Acetate-Glycine-Sucrose (“AGS”) formulation performed in the Hull (Hull Co./SP Industries, Warminster, Pa.) clinical lyophilizer. Filling volume is 4 ml in 10 ml tubing vials.

FIG. 3 illustrates exemplary lyophilization cycle for the protein at 25 mg/ml in Acetate-Mannitol-Sucrose (“AMS”) and Histidine-Mannitol-Sucrose (“HMS”) buffer. Cycle was performed on a Genesis (VirTis/SP Industries, Gardiner, N.Y.) laboratory lyophilizer. Fill volume is 4 ml in 10 ml vials.

FIG. 4 illustrates exemplary lyophilization cycle for the protein at 50 mg/ml in HMS buffer. Cycle was performed on a laboratory Genesis (VirTis/SP Industries, Gardiner, N.Y.) lyophilizer. Fill volume is 4 ml in 10 ml vials

FIG. 5 illustrates exemplary data showing the effect of protein concentration on crystallization of mannitol in HMS formulation. Mannitol crystallization peak can be seen during the ramp from −60° C. to −10° C. for protein concentrations up to 89 mg/ml. At a protein concentration between 96 and 115 mg/ml, mannitol crystallization occurs only during isothermal hold at −10° C.

FIG. 6 illustrates exemplary freeze-drying cycle for the protein at 100 mg/ml in HMS buffer.

FIG. 7 illustrates exemplary lyophilization cycle for the protein at 100 mg/ml in 10% sucrose, 5% sucrose+1% glycine, 5% sucrose+2.4% sorbitol formulations. All formulations contain 20 mM histidine.

FIG. 8 illustrates exemplary reconstitution of the protein at 100 mg/ml in 10% sucrose+20 mM histidine buffer. Water injection time was approximately 30 sec. Three minutes of constant swirling was used to dissolve the solids. Solution was cleared from effervescence in less than 30 sec.

FIG. 9 illustrates exemplary reconstitution of the protein at 100 mg/ml in 5% sucrose+1% glycine+20 mM histidine buffer. Water injection time was approximately 30 sec. After injection, water stayed on top of the cake without visible penetration to inside of the tablet. At least 9 minutes of constant swirling was used to dissolve the solids. No effervescence was detected during dissolution.

FIG. 10 illustrates exemplary reconstitution of the protein at 100 mg/ml in 5 sucrose+2.4% sorbitol+20 mM histidine buffer. Water injection time was approximately 30 sec. After injection, water stayed on top of the cake without visible penetration to inside of the tablet. At least 9 minutes of constant swirling was used to dissolve the solids. No effervescence was detected during dissolution.

FIG. 11 illustrates exemplary lyophilization cycle traces for formulation with low sucrose concentration the protein at 200 mg/ml in 5% sucrose, 10 mM histidine, 0.01% Polysorbate 80.

FIG. 12 illustrates exemplary lyophilization cycle traces for formulation with the protein concentration at 200 mg/ml in 10% sucrose, 10 mM histidine, 0.01% Polysorbate 80.

FIG. 13 illustrates an exemplary cake appearance of low (5%) and high (10%) sucrose in formulations containing the protein at 200 mg/ml.

FIG. 14 illustrates exemplary lyophilization cycle for the protein (baseline cycle).

FIG. 15 illustrates an exemplary cake appearance of lyophilized protein.

FIG. 16 illustrates Differential Scanning calorimetry (DSC) scan of lyophilized protein. Ramp rate was 2° C./min, ±0.5° C. modulations every 100 s.

FIG. 17 illustrates effect of pH and excipients on the protein liquid stability at accelerated temperatures.

FIG. 18 illustrates exemplary robustness study for the protein: cycle with elevated moisture.

FIG. 19 illustrates exemplary robustness study for the protein: “aggressive cycle” #4.

FIG. 20 illustrates an exemplary comparison of the cake appearance for lyophilized protein materials: half of cake was collapsed (aggressive cycle #4, right vial) versus intact cake (baseline cycle, left vial).

FIG. 21 illustrates DSC scan of the protein dry powder lyophilized using “aggressive” cycle #1 (Table 15). Ramp rate was 2° C./min, modulation±0.5° C. every 100 s. The shift in baseline on reversible signal (green) represents the glass transition, whereas the exothermic event on non-reversible signal (blue) represents apparent crystallization of some of the formulation components.

### DETAILED DESCRIPTION OF THE INVENTION

The present invention provides, among other things, lyophilized formulations for small modular immunopharmaceutical (SMIP™) proteins based on combinations of buffering agents, stabilizers, bulking agents, surfactants and/or other excipients. Lyophilized formulations according to the invention prevent proteins from freezing and dehydration stresses and preserve or enhance protein stability during freeze-drying and/or preserve or improve stability of lyophilized product during storage. The present invention also provides methods of preparing stable lyophilized formulations and uses thereof.

Various aspects of the invention are described in detail in the following sections. The use of sections is not meant to limit the invention. Each section can apply to any aspect of the invention. In this application, the use of “or” means “and/or” unless stated otherwise.

### Small Modular Immunopharmaceuticals

As used herein, a small modular immunopharmaceutical (SMIP™) protein refers to a protein that contains one or more of the following fused domains: a binding domain, an immunoglobulin hinge region or a domain derived therefrom, an immunoglobulin heavy chain C_{H2} constant region or a domain derived therefrom, and an immunoglobulin heavy chain C_{H3} constant region or a domain derived therefrom. SMIP™ protein therapeutics are preferably mono-specific (i.e., they recognize and attach to a single antigen target to initiate biological activity). The present invention also relates to multi-specific and/or multi-valent molecules such as SCORPION™ therapeutics, which incorporate a SMIP™ protein and also have an additional binding domain located C-terminally to the SMIP™ protein portion of the molecule. Preferably, the binding domains of SCORPION therapeutics each bind to a different target. The domains of small modular immunopharmaceuticals suitable for the present invention are, or are derived from, polypeptides that are the products of human gene sequences, any other natural or artificial sources, including genetically engineered and/or mutated polypeptides. Small modular immunopharmaceuticals are also known as binding domain-immunoglobulin fusion proteins.

In some embodiments, a hinge region suitable for a SMIP™ is derived from an immunoglobulin such as IgG1, IgG2, IgG3, IgG4, IgA, IgE, or the like. For example, a hinge region can be a mutant IgG1 hinge region polypeptide having either zero, one or two cysteine residues.

A binding domain suitable for a SMIP™ may be any polypeptide that possesses the ability to specifically recognize and bind to a cognate biological molecule, such as an antigen, a receptor (e.g., CD20), or complex of more than one molecule or assembly or aggregate.

Binding domains may include at least one immunoglobulin variable region polypeptide, such as all or a portion or fragment of a heavy chain or a light chain V-region, provided it is capable of specifically binding an antigen or other desired target structure of interest. In other embodiments, binding domains may include a single chain immunoglobulin-derived Fv product, which may include all or a portion of at least one immunoglobulin light chain V-region and all or a portion of at least one immunoglobulin heavy chain V-region, and which further comprises a linker fused to the V-regions.

The present invention can be applied to various small modular immunopharmaceuticals. Exemplary small modular immunopharmaceuticals may target receptors or other proteins, such as, CD3, CD4, CD8, CD19, CD20 and CD34; members of the HER receptor family such as the EGF receptor, HER2, HER3 or HER4 receptor; cell adhesion molecules such as LFA-1, Mol, p150, p95, VLA-4, ICAM-1, VCAM, growth factors such as VEGF; IgE; blood group antigens; flk2/flt3 receptor; obesity (OB) receptor; protein C; EGFR, RAGE, P40, Dkk1, NOTCH1, IL-13, IL-21, IL-4, and IL-22, etc.

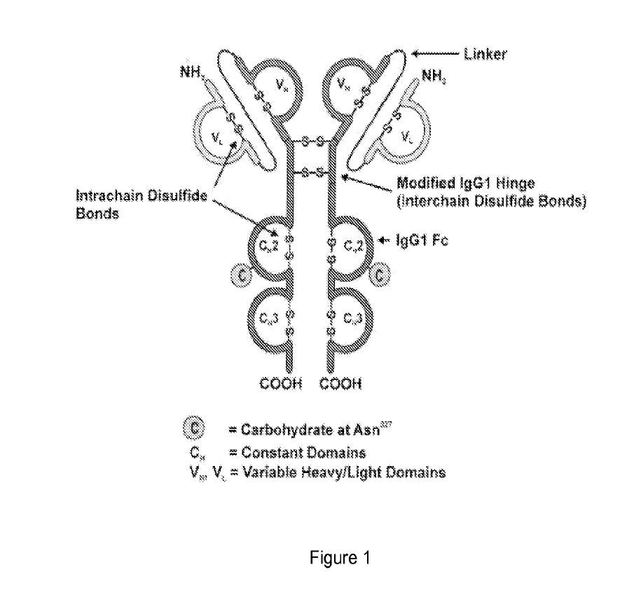

In some embodiments, the present invention is utilized to lyophilize or store small modular immunopharmaceuticals that specifically recognize CD20. An exemplary small modular immunopharmaceutical protein that specifically binds CD20 is shown in FIG. 1. As shown in FIG. 1, an anti-CD20 SMIP™ protein is typically a recombinant homodimeric fusion protein composed of three distinct domains: (1) a chimeric (murine/human) CD20 binding domain including the variable heavy (VH) and light (VL) chain fragments connected by a 15-amino acid linker; (2) a modified human IgG1 hinge domain and, (3) an IgG effector domain consisting of the CH2 and CH3 domains of human IgG1 (see FIG. 1).

Typically, a SMIP™ protein may exist in two distinctly associated homodimeric forms, the major form, which is the predicted interchain disulfide linked covalent homodimer (CD), and a homodimeric form that does not possess interchain disulfide bonds (dissociable dimer, DD). The dissociable dimer is generally fully active. Typically, a dimer has a theoretical molecular weight of approximately 106,000 daltons. SMIP™ proteins can also form multivalent complexes.

Typically, SMIP™ proteins are present as glycoproteins. For example, as shown in FIG. 1, an anti-CD20 SMIP™ protein may be modified with oligosaccharides at the N-linked glycosylation consensus sequence (e.g., ³²⁷NST) in the CH2 domain of each protein chain (see FIG. 1). SMIP™ proteins may also contain a core-fucosylated asialo-agalacto-biantennary N-linked oligosaccharide (G0F); COOH-terminal Gly⁴⁷⁶, and NH2-terminal pyroglutamate on each chain. Two minor glycoforms, G1F/G0F and G1F/G1F, and other expected trace-level N-linked glycoforms may also present. Additionally, low levels of a Core 1 O-glycan modification is also observed in the hinge region of SMIP™ proteins.

In some embodiments, the isoelectric point (pI or IEP) of SMIP™ proteins ranges from approximately 7.0 to 9.0 (e.g., 7.2, 7.4, 7.6, 7.8, 8.0, 8.2, 8.4, 8.6, 8.8).

The present invention can be used to formulate SMIP™ proteins in various forms as discussed herein (e.g., monomeric polypeptide, homodimer, dissociable dimer or multivalent complexes). The present invention can be used to formulate various modified SMIP™ proteins, such as humanized SMIP™, or chimeric SMIP™ proteins. As used herein, the term “humanized SMIP™ proteins” refers to SMIP™ proteins that include at least one humanized immunoglobulin region (e.g., humanized immunoglobulin variable or constant region). In some embodiments, a humanized SMIP™ protein comprises a humanized variable region that includes a variable framework region derived substantially from a human immunoglobulin (e.g., a fully human FR1, FR2, FR3, and/or FR4), while maintaining target-specific one or more complementarity determining regions (CDRs) (e.g., at least one CDR, two CDRs, or three CDRs). In some embodiments, a humanized SMIP™ protein comprises one or more human or humanized constant regions (e.g., human immunoglobulin C_{H2} and/or C_{H3} domains). The term “substantially from a human immunoglobulin or antibody” or “substantially human” means that, when aligned to a human immunoglobulin or antibody amino sequence for comparison purposes, the region shares at least 80-90%, preferably 90-95%, more preferably 95-99% identity (i.e., local sequence identity) with the human framework or constant region sequence, allowing, for example, for conservative substitutions, consensus sequence substitutions, germline substitutions, backmutations, and the like. As used herein, the term “chimeric SMIP™ proteins” refers to SMIP™ proteins whose variable regions derive from a first species and whose constant regions derive from a second species. Chimeric SMIP™ proteins can be constructed, for example by genetic engineering, from immunoglobulin gene segments belonging to different species. Humanized and chimeric SMIP™ proteins are further described in International Application Publication No. WO 2008/156713, which is incorporated by reference herein.

The present invention can also be used to formulate SMIP™ proteins with modified glycosylation patterns and/or mutations to the hinge, C_{H2} and/or C_{H3} domains that alter the effector functions. In some embodiments, SMIP™ proteins may contain mutations on adjacent or close sites in the hinge link region that affect affinity for receptor binding. In addition, the invention can be used to formulate fusion proteins including a small modular immunopharmaceutical polypeptide or a portion thereof.

In some embodiments, the present invention can be used to formulate SMIP™ proteins that include an amino acid sequence of any one of SEQ ID NOs:1-76 (see the Exemplary SMIP™ Sequences section), or a variant thereof. In some embodiments, the present invention can be used to formulate SMIP™ proteins that contain a variable domain having an amino acid sequence of any one of SEQ ID NOs:1-59 or a variant thereof. In some embodiments, the present invention can be used to formulate SMIP™ proteins that contain a variable domain having an amino acid sequence of any one of SEQ ID NOs:1-59 or a variant thereof, a hinge region having an amino acid sequence of any one of SEQ ID NOs:60-64 or a variant thereof, and/or an immunoglobulin constant region having an amino acid sequence of SEQ ID NO:65 or 66 or a variant thereof. In some embodiments, the present invention can be used to formulate SMIP™ proteins that have an amino acid sequence of any one of SEQ ID NOs:67-76, or a variant thereof.

As used herein, variants of a parent sequence include, but are not limited to, amino acid sequences that are at least 70%, 75%, 80%, 85%, 90%, 95%, 96%, 97%, 98%, 99%, identical to the parent sequence. The percent identity of two amino acid sequences can be determined by visual inspection and mathematical calculation, or more preferably, the comparison is done by comparing sequence information using a computer program such as the Genetics Computer Group (GCG; Madison, Wis.) Wisconsin package version 10.0 program, “GAP” (Devereux et al., 1984, Nucl. Acids Res. 12: 387) or other comparable computer programs. The preferred default parameters for the ‘GAP’ program includes: (1) the weighted amino acid comparison matrix of Gribskov and Burgess ((1986), Nucl. Acids Res. 14: 6745), as described by Schwartz and Dayhoff, eds., Atlas of Polypeptide Sequence and Structure, National Biomedical Research Foundation, pp. 353-358 (1979), or other comparable comparison matrices; (2) a penalty of 30 for each gap and an additional penalty of 1 for each symbol in each gap for amino acid sequences; (3) no penalty for end gaps; and (4) no maximum penalty for long gaps. Other programs used by those skilled in the art of sequence comparison can also be used.

Additional small modular immunopharmaceuticals are further described in, e.g., US Patent Publications 20030133939, 20030118592, 20040058445, 20050136049, 20050175614, 20050180970, 20050186216, 20050202012, 20050202023, 20050202028, 20050202534, 20050238646, and 20080213273; International Patent Publications WO 02/056910, WO 2005/037989, and WO 2005/017148, which are all incorporated by reference herein.

### Lyophilized Formulations for Small Modular Immunopharmaceuticals

Lyophilization, or freeze-drying, is a commonly employed technique for preserving proteins which serves to remove water from the protein preparation of interest. Lyophilization, is a process by which the material to be dried is first frozen and then the ice or frozen solvent is removed by sublimation in a vacuum environment.

Lyophilization generally includes three main stages: freezing, primary drying and secondary drying. Freezing is necessary to convert water to ice or some amorphous formulation components to the crystalline form. Primary drying is the process step when ice is removed from the frozen product by direct sublimation at low pressure and temperature. Secondary drying is the process step when bounded water is removed from the product matrix utilizing the diffusion of residual water to the evaporation surface. Product temperature during secondary drying is normally higher than during primary drying. See, Tang X. et al. (2004) “Design of freeze-drying processes for pharmaceuticals: Practical advice,” Pharm. Res., 21:191-200; Nail S. L. et al. (2002) “Fundamentals of freeze-drying,” in Development and manufacture of protein pharmaceuticals. Nail SL editors. New York: Kluwer Academic/Plenum Publishers, pp 281-353; Wang et al. (2000) “Lyophilization and development of solid protein pharmaceuticals,” Int. J. Pharm., 203:1-60; Williams N A et al. (1984) “The lyophilization of pharmaceuticals; A literature review.” J. Parenteral Sci. Technol., 38:48-59.

Because of the variations in temperature and pressure through the lyophilization process, an appropriate choice of excipients or other components such as stabilizers, buffering agents, bulking agents, and surfactants are needed to prevent SMIP™ from degradation (e.g., protein aggregation, deamidation, and/or oxidation) during freeze-drying and storage.

Thus, the present invention provides stable lyophilized formulations containing SMIP™ based on combinations of stabilizers, buffering agents, bulking agents, and/or other excipients. As used herein, a “stable” formulation is one in which the protein therein essentially retains its physical and chemical stability and integrity during lyophilization and upon storage. Various analytical techniques for measuring protein stability are available in the art and are reviewed in Peptide and Protein Drug Delivery, 247-301, Vincent Lee Ed., Marcel Dekker, Inc., New York, N.Y., Pubs. (1991) and Jones, A. Adv. Drug Delivery Rev. 10: 29-90 (1993). Stability can be measured after storage at a selected temperature (e.g., 0° C., 5° C., 25° C. (room temperature), 30° C., 40° C.) for a selected time period (e.g., 2 weeks, 1 month, 1.5 months, 2 months, 3, months, 4 months, 5 months, 6 months, 12 months, 18 months, 24 months, etc.). For rapid screening, the formulation may be kept at 40° C. for 2 weeks to 1 month, at which time stability is measured. Where the formulation is to be stored at 2-8° C., generally the formulation should be stable at 25° C. (i.e., room temperature) or 40° C. for at least 1 month and/or stable at 2-8° C. for at least 3 months, 6 months, 1 year or 2 years. Where the formulation is to be stored at 30° C., generally the formulation should be stable for at least 3 months, 6 months, 1 year or 2 years at 30° C. and/or stable at 40° C. for at least 2 weeks, 1 month, 3 months or 6 months. In some embodiments, the extent of aggregation following lyophilization and storage can be used as an indicator of protein stability (see Examples herein). As used herein, the term “high molecular weight (“HMW”) aggregates” refers to an association of at least two protein monomers. For the purposes of this invention, a monomer refers to the single unit of any biologically active form of the protein of interest. For example, a monomer of a small modular immunopharmaceutical protein can be a monomeric polypeptide, or a homodimer, or a dissociable dimer, or a unit of multivalent complex of SMIP™ protein. The association may be covalent, non-covalent, disulfide, non-reducible crosslinking, or by other mechanism.

For example, a “stable” formulation may be one wherein less than about 10% (e.g., less than 9%, 8%, 7%, 6%, 5%, 4%, 3%, 2%, 1%, 0.5%) and preferably less than about 5% (e.g., less than 4%, 3%, 2%, 1%, 0.5%) of the protein is present as an aggregate in the formulation (also referred to as high molecular weight species (“HMW”)). In some embodiments, stability can be measured by an increase in aggregate formation following lyophilization and storage of the lyophilized formulation. For example, a “stable” lyophilized formulation may be one wherein the increase in aggregate in the lyophilized formulation is less than about 5% (e.g., less than 4%, 3%, 2%, 1%, 0.5%) and preferably less than about 3% (e.g., 2%, 1%, 0.5%, 0.2%, 0.1%) when the lyophilized formulation is stored at 25° C. (i.e., room temperature) or 40° C. for at least 2 weeks, 1 month, 3 months or 6 months, or at 2-8° C. for at least 3 months, 6 months, 1 year or 2 years. Aggregate or HMW species can be analyzed using methods known in the art including, but not limited to, size exclusion HPLC (SE-HPLC), cation exchange-HPLC (CEX-HPLC), reversed phase HPLC(RP-HPLC), multi-angle light scattering (MALS), fluorescence, ultraviolet absorption, nephelometry, capillary electrophoresis (CE), SDS-PAGE, and combinations thereof.

In some embodiments, stability of the protein formulation may be measured using a biological activity assay. For example, a “stable” formulation may be one that retains at 80% (e.g., 85%, 90%, 92%, 94%, 96%, 98%, or 99%) of the original protein activity after lyophilization or storage at a selected temperature (e.g., 0° C., 5° C., 25° C. (room temperature), 30° C., 40° C.) for a selected time period (e.g., 2 weeks, 1 month, 1.5 months, 2 months, 3, months, 4 months, 5 months, 6 months, 12 months, 18 months, 24 months, etc.). Biological activity assays of SMIP™ are known in the art. Exemplary methods are described in US Patent Publications 20030133939, 20030118592, 20050136049, and 20080213273; International Patent Publications WO 02/056910, WO 2005/037989, and WO 2005/017148, which are all incorporated by reference herein.

### Preparation of Formulations

SMIP™ proteins to be formulated can be prepared using techniques which are well established in the art including, but not limited to, recombinant techniques and peptide synthesis or a combination of these techniques. SMIP™ proteins can be obtained from any in vivo or in vitro protein expression systems including, but not limited to, product-producing recombinant cells, bacteria, fungal cells, insect cells, transgenic plants or plant cells, transgenic animals or animal cells, or serum of animals, ascites fluid, hybridoma or myeloma supernatants. Suitable bacterial cells include, but are not limited to, Escherichia coli cells. Examples of suitable E. coli strains include: HB101, DH5α, GM2929, JM109, KW251, NM538, NM539, and any E. coli strain that fails to cleave foreign DNA. Suitable fungal host cells that can be used include, but are not limited to, Saccharomyces cerevisiae, Pichia pastoris and Aspergillus cells. Suitable insect cells include, but are not limited to, S2 Schneider cells, D. Mel-2 cells, SF9, SF21, High-5™, Mimic-SF9, MG1 and KC1 cells. Suitable exemplary recombinant cell lines include, but are not limited to, BALB/c mouse myeloma line, human retinoblasts (PER.C6), monkey kidney cells, human embryonic kidney line (293), baby hamster kidney cells (BHK), Chinese hamster ovary cells (CHO), mouse sertoli cells, African green monkey kidney cells (VERO-76), human cervical carcinoma cells (HeLa), canine kidney cells, buffalo rat liver cells, human lung cells, human liver cells, mouse mammary tumor cells, TRI cells, MRC 5 cells, FS4 cells, and human hepatoma line (Hep G2).

SMIP™ proteins can be expressed using various vectors (e.g., viral vectors) known in the art and cells can be cultured under various conditions known in the art (e.g., fed-batch). Various methods of genetically engineering cells to produce proteins are well known in the art. See e.g., Ausabel et al., eds. (1990), Current Protocols in Molecular Biology (Wiley, New York). Exemplary methods are described in US Patent Publications 20030133939, 20030118592, 20050136049, and 20080213273; International Patent Publications WO 02/056910, WO 2005/037989, and WO 2005/017148, which are all incorporated by reference herein.

After preparation of a SMIP™ of interest, a “pre-lyophilized formulation” (also referred to as “a formulation for lyophilization”) can be produced. The amount of SMIP™ present in the pre-lyophilized formulation is determined taking into account the desired dose volumes, mode(s) of administration etc.

Suitable formulations for lyophilization may contain a SMIP™ of interest at various concentrations. In some embodiments, formulations suitable for lyophilization may contain a protein of interest at a concentration in the range of about 1 mg/ml to 400 mg/ml (e.g., about 1 mg/ml to 50 mg/ml, 1 mg/ml to 60 mg/ml, 1 mg/ml to 70 mg/ml, 1 mg/ml to 80 mg/ml, 1 mg/ml to 90 mg/ml, 1 mg/ml to 100 mg/ml, 100 mg/ml to 150 mg/ml, 100 mg/ml to 200 mg/ml, 100 mg/ml to 250 mg/ml, 100 mg/ml to 300 mg/ml, 100 mg/ml to 350 mg/ml, 100 mg/ml to 400 mg/ml, 25 mg/ml to 350 mg/ml, 25 mg/ml to 400 mg/ml, 25 mg/ml to 250 mg/ml, 25 mg/ml to 200 mg/ml, 50 mg/ml to 200 mg/ml, 25 mg/ml to 150 mg/ml). In some embodiments, formulations suitable for lyophilization may contain a protein of interest at a concentration of approximately 25 mg/ml, 50 mg/ml, 75 mg/ml, 100 mg/ml, 125 mg/ml, 150 mg/ml, 175 mg/ml, 200 mg/ml, 250 mg/ml, 300 mg/ml, 350 mg/ml or 400 mg/ml.

The protein is generally present in solution. For example, SMIP™ proteins may be present in a pH-buffered solution at a pH from about 4-8 (e.g., 4.0, 4.5, 5.0, 5.5, 6.0, 6.5, 7.0, 7.5, and 8.0) and, in some embodiments, from about 5-7. Exemplary buffers include histidine, phosphate, tris(hydroxymethyl)aminomethane (“Tris”), citrate, acetate, sodium acetate, phosphate, succinate and other organic acids. The buffer concentration can be from about 1 mM to about 30 mM, or from about 3 mM to about 20 mM, depending, for example, on the buffer and the desired isotonicity of the formulation (e.g., of the reconstituted formulation). In some embodiments, a suitable buffering agent is present at a concentration of approximately 1 mM, 5 mM, 10 mM, 15 mM, 20 mM, 25 mM, 30 mM, or 50 mM.

In some embodiments, formulations suitable for lyophilization may contain a stabilizing agent to protect the protein. A stabilizing agent is also referred to as a lyoprotectant. Typically, a suitable stabilizing agent is a non-reducing sugar such as sucrose, raffinose, trehalose, or amino acids such as glycine, arginine and methionine. The amount of stabilizing agent or lyoprotectant in the pre-lyophilized formulation is generally such that, upon reconstitution, the resulting formulation will be isotonic. However, hypertonic reconstituted formulations may also be suitable. In addition, the amount of lyoprotectant must not be too low such that an unacceptable amount of degradation/aggregation of the SMIP™ occurs upon lyophilization. Where the lyoprotectant is a sugar (such as sucrose or trehalose) and the protein is a SMIP™, exemplary lyoprotectant concentrations in the pre-lyophilized formulation may range from about 10 mM to about 400 mM (e.g., from about 30 mM to about 300 mM, and from about 50 mM to about 100 mM), or alternatively, from 0.5% to 15% (e.g., from 1% to 10%, from 5% to 15%, from 5% to 10%) by weight. In some embodiments, the ratio of the mass amount of the stabilizing agent and the SMIP™ is about 1:1. In other embodiments, the ratio of the mass amount of the stabilizing agent and the SMIP™ can be about 0.1:1, 0.2:1, 0.25:1, 0.4:1, 0.5:1, 1:1, 2:1, 2.6:1, 3:1, 4:1, 5:1, 10:1, or 20:1.

In some embodiments, suitable formulations for lyophilization may further include one or more bulking agents. A “bulking agent” is a compound which adds mass to the lyophilized mixture and contributes to the physical structure of the lyophilized cake. For example, a bulking agent may improve the appearance of lyophilized cake (e.g., essentially uniform lyophilized cake). Suitable bulking agents include, but are not limited to, sodium chloride, lactose, mannitol, glycine, sucrose, trehalose, hydroxyethyl starch. Exemplary concentrations of bulking agents are from about 1% to about 10% (e.g., 1.0%, 1.5%, 2.0%, 2.5%, 3.0%, 3.5%, 4.0%, 4.5%, 5.0%, 5.5%, 6.0%, 6.5%, 7.0%, 7.5%, 8.0%, 8.5%, 9.0%, 9.5%, and 10.0%).

In some embodiments, formulations for lyophilization contain an isotonicity agent to keep the pre-lyophilization formulations or the reconstituted formulations isotonic. Typically, by “isotonic” is meant that the formulation of interest has essentially the same osmotic pressure as human blood. Isotonic formulations will generally have an osmotic pressure from about 240 mOsm/kg to about 350 mOsm/kg. Isotonicity can be measured using, for example, a vapor pressure or freezing point type osmometers. Exemplary isotonicity agents include, but are not limited to, glycine, sorbitol, mannitol, sodium chloride and arginine. In some embodiments, suitable isotonic agents may be present in pre-lyophilized formulations at a concentration from about 0.01-5% (e.g., 0.05, 0.1, 0.15, 0.2, 0.3, 0.4, 0.5, 0.75, 1.0, 1.25, 1.5, 2.0, 2.5, 3.0, 4.0 or 5.0%) by weight.

In some embodiments, it is desirable to add a surfactant to formulations for lyophilization. Exemplary surfactants include nonionic surfactants such as Polysorbates (e.g., Polysorbates 20 or 80); poloxamers (e.g., poloxamer 188); Triton; sodium dodecyl sulfate (SDS); sodium laurel sulfate; sodium octyl glycoside; lauryl-, myristyl-, linoleyl-, or stearyl-sulfobetaine; lauryl-, myristyl-, linoleyl- or stearyl-sarcosine; linoleyl-, myristyl-, or cetyl-betaine; lauroamidopropyl-, cocamidopropyl-, linoleamidopropyl-, myristamidopropyl-, palmidopropyl-, or isostearamidopropyl-betaine (e.g., lauroamidopropyl); myristamidopropyl-, palmidopropyl-, or isostearamidopropyl-dimethylamine; sodium methyl cocoyl-, or disodium methyl ofeyl-taurate; and the MONAQUAT™ series (Mona Industries, Inc., Paterson, N.J.), polyethyl glycol, polypropyl glycol, and copolymers of ethylene and propylene glycol (e.g., Pluronics, PF68, etc). Typically, the amount of surfactant added is such that it reduces aggregation of the reconstituted protein and minimizes the formation of particulates or effervescences after reconstitution. For example, a surfactant may be present in a pre-lyophilized formulation at a concentration from about 0.001-0.5% (e.g., about 0.005-0.05%, or 0.005-0.01%). In particular, a surfactant may be present in a pre-lyophilized formulation at a concentration of approximately 0.005%, 0.01%, 0.02%, 0.1%, 0.2%, 0.3%, 0.4%, or 0.5%, etc. Alternatively, or in addition, the surfactant may be added to the lyophilized formulation and/or the reconstituted formulation.

In certain embodiments, a mixture of a stabilizing agent (such as sucrose or trehalose) and a bulking agent (e.g., mannitol or glycine) is used in the preparation of the pre-lyophilization formulation. In certain embodiments of the invention, a mixture of a stabilizing agent (such as sucrose or trehalose), a bulking agent (e.g., mannitol or glycine) and a surfactant (e.g., Polysorbate 80) is used in the preparation of the pre-lyophilization formulation.

Other pharmaceutically acceptable carriers, excipients or stabilizers such as those described in Remington's Pharmaceutical Sciences 16th edition, Osol, A. Ed. (1980) may be included in the pre-lyophilized formulation (and/or the lyophilized formulation and/or the reconstituted formulation) provided that they do not adversely affect the desired characteristics of the formulation. Acceptable carriers, excipients or stabilizers are nontoxic to recipients at the dosages and concentrations employed and include, but are not limited to, additional buffering agents; preservatives; co-solvents; antioxidants including ascorbic acid and methionine; chelating agents such as EDTA; metal complexes (e.g., Zn-protein complexes); biodegradable polymers such as polyesters; and/or salt-forming counterions such as sodium.

Formulations described herein may contain more than one protein as appropriate for a particular indication being treated, preferably those with complementary activities that do not adversely affect the other protein.

Formulations to be used for in vivo administration must be sterile. This is readily accomplished by filtration through sterile filtration membranes, prior to, or following, lyophilization and reconstitution.

After the protein, stabilizing agent and other optional components are mixed together, the formulation is lyophilized. Many different freeze-dryers are available for this purpose such as Hull pilot scale dryer (SP Industries, USA), Genesis (SP Industries) laboratory freeze-dryers, or any freeze-dryers capable of controlling the given lyophilization process parameters. Freeze-drying is accomplished by freezing the formulation and subsequently subliming ice from the frozen content at a temperature suitable for primary drying. Initial freezing brings the formulation to a temperature below about −20° C. (e.g., −50° C., −45° C., −40° C., −35° C., −30° C., −25° C., etc.) in typically not more than about 4 hours (e.g., not more than about 3 hours, not more than about 2.5 hours, not more than about 2 hours). Under this condition, the product temperature is typically below the eutectic point or the collapse temperature of the formulation. Typically, the shelf temperature for the primary drying will range from about −30 to 25° C. (provided the product remains below the melting point during primary drying) at a suitable pressure, ranging typically from about 20 to 250 mTorr. The formulation, size and type of the container holding the sample (e.g., glass vial) and the volume of liquid will mainly dictate the time required for drying, which can range from a few hours to several days. A secondary drying stage is carried out at about 0-60° C., depending primarily on the type and size of container and the type of SMIP™ employed. Again, volume of liquid will mainly dictate the time required for drying, which can range from a few hours to several days.

Optionally, an annealing step may be introduced during the initial freezing of the product. The annealing step may reduce the overall cycle time. Without wishing to be bound by any theories, it is contemplated that the annealing step can help promote excipient, particularly mannitol, crystallization, which, in turn, increases the glass transition temperature for the remaining amorphous components of the formulation, allowing for higher shelf temperatures. The annealing step includes an interval or oscillation in the temperature during freezing. For example, the freeze temperature may be −40° C., and the annealing step will increase the temperature to, for example, −10° C. and maintain this temperature for a set period of time. The annealing step time may range from 0.5 hours to 8 hours (e.g., 0.5, 1.0 1.5, 2.0, 2.5, 3, 4, 6, and 8 hours). The annealing temperature may be between the freezing temperature and 0° C.

Lyophilized product in accordance with the present invention can be assessed based on product quality analysis, reconstitution time, quality of reconstitution, high molecular weight, moisture, and glass transition temperature. Typically, protein quality and dry product analysis include product degradation rate analysis using methods including, but not limited to, size exclusion HPLC (SE-HPLC), cation exchange-HPLC (CEX-HPLC), X-ray diffraction (XRD), modulated differential scanning calorimetry (mDSC), reversed phase HPLC(RP-HPLC), multi-angle light scattering (MALS), fluorescence, ultraviolet absorption, nephelometry, capillary electrophoresis (CE), SDS-PAGE, and combinations thereof. In some embodiments, evaluation of lyophilized product in accordance with the present invention include a step of evaluating cake appearance. However, in some embodiments, evaluation of lyophilized product in accordance with the present invention does not include a step of evaluating cake appearance.

Lyophilization may be performed in a container, such as a tube, a bag, a bottle, a tray, a vial (e.g., a glass vial), syringe or any other suitable containers. The containers may be disposable. Lyophilization may also be performed in a large scale or small scale. In some instances, it may be desirable to lyophilize the protein formulation in the container in which reconstitution of the protein is to be carried out in order to avoid a transfer step. The container in this instance may, for example, be a 3, 4, 5, 10, 20, 50 or 100 cc vial.

As a general proposition, lyophilization will result in a lyophilized formulation in which the moisture content thereof is less than about 5%, less than about 4%, less than about 3%, less than about 2%, less than about 1%, and less than about 0.5%.

Examples of SMIP™ formulations according to the present invention include the following:

1. 25 mg/ml SMIP™ (e.g., TRU-015) in 6.5% sucrose, 50 mM glycine, 20 mM sodium acetate, pH6.0.

2. 50 mg/ml SMIP™ (e.g., TRU-015) in 20 mM histidine, 4% mannitol, 1% sucrose, pH 6.0.

3. 100 mg/ml SMIP™ (e.g., TRU-015) in 20 mM histidine, 4% mannitol, 1% sucrose, pH 6.0.

4. 100 mg/ml SMIP™ in 10% sucrose, 20 mM histidine, 0.01% Polysorbate-80.

5. 100 mg/ml SMIP™ in 5% sucrose, 1% glycine, 20 mM histidine, 0.01% Polysorbate-80.

6. 100 mg/ml SMIP™ in 5% sucrose, 2.4% sorbitol, 20 mM histidine, 0.01% Polysorbate-80.

7. 200 mg/ml SMIP™ in 5% or 10% sucrose, 20 mM histidine, 0.01% Polysorbate-80.

Additional exemplary formulations are described in the Example sections.

### Storage of Lyophilized Formulations

Generally, lyophilized products can be stored for extended periods of time at room temperature. Storage temperature may typically range from 0° C. to 45° C. (e.g., 4° C., 20° C., 25° C., 45° C. etc.). Lyophilized product may be stored for a period of months to a period of years. Storage time generally will be 24 months, 12 months, 6 months, 4.5 months, 3 months, 2 months or 1 month. Lyophilized product can be stored directly in the lyophilization container, which may also function as the reconstitution vessel, eliminating transfer steps. Alternatively, lyophilized product formulations may be measured into smaller increments for storage. Storage should generally avoid circumstances that lead to degradation of the proteins, including but not limited to exposure to sunlight, UV radiation, other forms of electromagnetic radiation, excessive heat or cold, rapid thermal shock, and mechanical shock.

### Reconstitution of Lyophilized Formulations

At the desired stage, typically when it is time to administer the protein to the patient, the lyophilized formulation may be reconstituted with a diluent such that the protein concentration in the reconstituted formulation is desirable. For example, a SMIP™ protein can be present in a reconstituted formulation at a concentration of at least 25 mg/ml (e.g., from about 25 mg/ml to about 400 mg/ml). In various embodiments, the protein concentration of the reconstituted formulation is at least 25 mg/ml, at least 50 mg/ml, at least 75 mg/ml, at least 100 mg/ml, at least 150 mg/ml, at least 200 mg/ml, at least 250 mg/ml, at least 300 mg/ml or at least 400 mg/ml. High protein concentrations in the reconstituted formulation are considered to be particularly useful where subcutaneous or intramuscular delivery of the reconstituted formulation is intended. However, for other routes of administration, such as intravenous administration, lower concentrations of the protein in the reconstituted formulation may be desired (for example from about 5-50 mg/ml, or from about 10-40 mg/ml protein in the reconstituted formulation).

Reconstitution generally takes place at a temperature of about 25° C. to ensure complete hydration, although other temperatures may be employed as desired. The time required for reconstitution will depend, e.g., on the type of diluent, amount of excipient(s) and protein. Exemplary diluents include sterile water, bacteriostatic water for injection (BWFI), a pH buffered solution (e.g., phosphate-buffered saline), sterile saline solution, Ringer's solution or dextrose solution. Suitable diluents may optionally contain a preservative. Exemplary preservatives include aromatic alcohols such as benzyl or phenol alcohol. The amount of preservative employed is determined by assessing different preservative concentrations for compatibility with the protein and preservative efficacy testing. For example, if the preservative is an aromatic alcohol (such as benzyl alcohol), it can be present in an amount from about 0.1-2.0%, from about 0.5-1.5%, or about 1.0-1.2%.

### Administration of Reconstituted Formulations

The reconstituted formulation is administered to a subject in need of treatment with the protein (e.g., a small modular immunopharmaceutical protein), for example, a human, in accordance with known methods, such as intravenous administration as a bolus or by continuous infusion over a period of time, by intramuscular, intraperitoneal, intracerebrospinal, subcutaneous, intra-articular, intrasynovial, intrathecal, oral, topical, or inhalation routes.

In some embodiments, the reconstituted formulation is administered to the subject by subcutaneous (i.e., beneath the skin) administration. For such purposes, the formulation may be injected using a syringe. However, other devices for administration of the formulation are available such as injection devices (e.g., the Inject-ease™ and Genject™ devices); injector pens (such as the GenPen™); needleless devices (e.g., MediJector™ and BioJector™); and subcutaneous patch delivery systems.

The appropriate dosage (“therapeutically effective amount”) of the small modular immunopharmaceutical will depend, for example, on the condition to be treated, the severity and course of the condition, whether the protein is administered for preventive or therapeutic purposes, previous therapy, the patient's clinical history and response to the protein, the type of protein used, and the discretion of the attending physician. The small modular immunopharmaceutical is suitably administered to the patient at one time or over a series of treatments and may be administered to the patient at any time from diagnosis onwards. The protein may be administered as the sole treatment or in conjunction with other drugs or therapies useful in treating the condition in question.

### Kits

The present invention provides kits or other articles of manufacture which contains the lyophilized formulation of the present invention and provides instructions for its reconstitution and/or use. Kits or other articles of manufacture may include a container. Suitable containers include, for example, bottles, vials, and syringes. The container may be formed from a variety of materials such as glass or plastic. The container holds the lyophilized formulation and the label on, or associated with, the container may indicate directions for reconstitution and/or use. For example, the label may indicate that the lyophilized formulation is reconstituted to protein concentrations as described above. The label may further indicate that the formulation is useful or intended for, for example, subcutaneous administration. The container holding the formulation may be a multi-use vial, which allows for repeat administrations (e.g., from 2-6 administrations) of the reconstituted formulation. Kits or other articles of manufacture may further include a second container comprising a suitable diluent (e.g., BWFI). Upon mixing of the diluent and the lyophilized formulation, the final protein concentration in the reconstituted formulation will generally be at least 25 mg/ml (e.g., at least 25 mg/ml, at least 50 mg/ml, at least 75 mg/ml, at least 100 mg/ml, at least 150 mg/ml, at least 200 mg/ml, at least 250 mg/ml at least 300 mg/ml, or at least 400 mg/ml). Kits or other articles of manufacture may further include other materials desirable from a commercial and user standpoint, including other buffers, diluents, filters, needles, syringes, and package inserts with instructions for use.

In some embodiments, a kit according to the invention includes a vial or other suitable container containing lyophilized SMIP™ protein and a pre-filled diluent syringe. The pre-filled diluent may be any solution suitable for reconstitution (e.g., BWFI, or 0.9% Sodium Chloride solution, etc.). A suitable syringe may be plastic or glass and may be disposable or re-usable. A suitable syringe may also be of various sizes (e.g., 1 ml, 2 ml, 4 ml, 6 ml, 8 ml, 10 ml). In some embodiments, a syringe may have a plunger rod attached to the syringe tube. In some embodiments, a syringe may have a detached plunger rod that need to be assembled by the user. Typically, a suitable syringe may have a tamper-resistant plastic tip cap that can be taken or broken off before administration. The cap may also be replaced to prevent possible contamination if the reconstituted SMIP™ protein is not immediately used. Suitable vials or other containers containing lyophilized SMIP™ product may be plastic or glass and may be disposable or re-usable. A suitable vial or other container such as an ampoule may be sealed with, e.g., rubber stopper, glass and/or plastic cap. In some embodiments, a kit may include an adapter that can be used to penetrate the vial stopper. In some embodiments, an adapter includes a needle that can be used to penetrate the vial stopper and is adapted to be attached to the syringe for reconstitution of the lyophilized product and injection. In some embodiments, a kit may include multiple prefilled vials, multiple pre-filled syringes, and/or a larger syringe for administering the contents of multiple vials. Typically, components of a kit can be separately packaged and sterilized. In some embodiments, a kit may include an instruction for use including specific reconstitution and/or administration procedures.

The invention will be more fully understood by reference to the following examples. They should not, however, be construed as limiting the scope of the invention. All literature citations are incorporated by reference.

### EXAMPLES

### Example 1

### Acetate-Glycine-Sucrose (“AGS”) Lyophilization Formulation Containing 25 mg/ml TRU-015

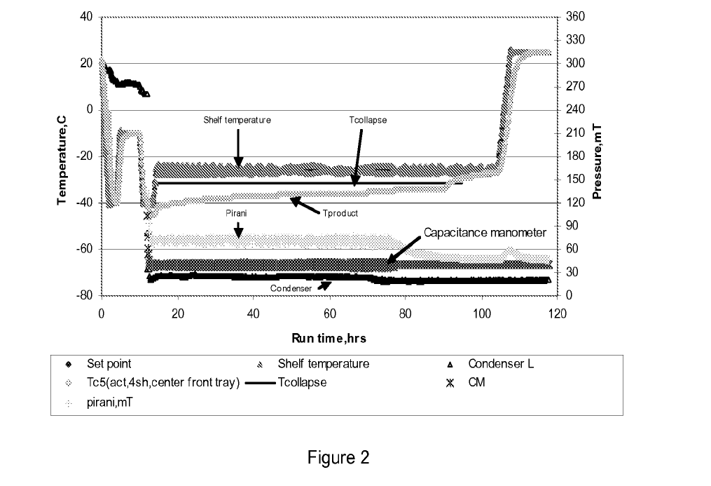

In this example, an AGS formulation was designed for lyophilizing TRU-015 at a concentration of approximately 25 mg/ml. Specifically, an AGS formulation used in this example included 6.5% sucrose, 50 mM glycine, 20 mM sodium acetate at pH 6.0. The protein concentration was 25 mg/ml, giving 100 mg of protein per vial. Sucrose serves as a stabilizer and bulking agent, glycine was added as stabilizer and isotonicity agent. Sodium acetate is the buffer. AGS formulation had a glass transition of −34.2° C. measured by Modulated Differential Scanning calorimeter (“DSC”). The collapse temperature of AGS formulation, as measured by Freeze-Drying Microscope (“FDM”), was found to be −31.4° C. The total lyophilization process in a laboratory scale lyophilizer lasted about 120 hours. An optional annealing step at −10° C. resulted in a decreased cycle time of 90 hours at laboratory scale. The lyophilization cycle was scaled up to run in a GMP clinical facility. The clinical scale lyophilization total cycle time was approximately 117 hours. An exemplary lyophilization program and exemplary cycle traces are shown in Table 1 and FIG. 2.

TABLE 1
Exemplary lyophilization program for
25 mg/ml TRU-015 in AGS formulation

| | | | |
|---|---|---|---|
| Step | | | Total cycle |
| # | Step description | Pressure, mT | time, hrs |

Freezing

| | | | |
|---|---|---|---|
| 1 | Ramp from 22° C. to −40° C. | Atmosphere | 2.00 |
| | in 120 min | | |
| 2 | Hold at −40° C. for 120 min | Atmosphere | 4.00 |
| 3 | Ramp from −40° C. to −10° C. | Atmosphere | 5.00 |
| | in 60 min | | |
| 4 | Hold at −10° C. for 300 min | Atmosphere | 10.00 |
| 5 | Ramp from −10° C. to −40° C. | Atmosphere | 11.00 |
| | in 60 min | | |
| 6 | Hold at −40° C. for 60 min | Atmosphere | 12.00 |
| 7 | Vacuum initiating | 40 | 13.00 |

Primary drying

| | | | |
|---|---|---|---|
| 8 | Ramp from −40° C. to −26° C. | 40 | 13.50 |
| | in 30 min | | |
| 9 | Hold at −26° C. for 5400 min | 40 | 103.50 |

Secondary drying

| | | | |
|---|---|---|---|
| 10 | Ramp from −26° C. to 25° C. | 40 | 107.00 |
| | in 210 min | | |
| 11 | Hold at 25° C. for 600 min | 40 | 117.00 |

As shown in FIG. 2, the product temperature during primary drying was below collapse temperature (FIG. 2). The thermocouple and Pirani sensor indicated the completion of primary drying prior to the secondary drying ramp. This resulted in a good cake appearance and low residual moisture (1.2%). Glass transition temperature of the dry powder lyophilizate was 65° C. allowing storage at elevated temperature. Reconstitution time for the lyophilized product was less than 1 minute. Exemplary stability data are summarized in Table 2.

TABLE 2
Exemplary stability data of lyophilized TRU-015 in AGS formulation
(% high molecular weight (“HMW”) measured by SE-HPLC)

| Storage | High molecular weight species (HMW) by SE-HPLC, % | | | |
|---|---|---|---|---|
| temperature | To | 1 month | 2 months | 3 months |
| 5° C. | 2.6 | 2.7 | 2.7 | 2.7 |
| 25° C. | 2.6 | 2.7 | 2.8 | 2.7 |

This example suggests that the AGS formulation is suitable to preserve stability of the TRU-015 molecule. The exemplary lyophilization cycle described herein is suitable for lyophilizing TRU-015 in AGS buffer.

### Example 2

### Acetate-Mannitol-Sucrose (“AMS”) or Histidine-Mannitol-Sucrose (“HMS”) Formulations

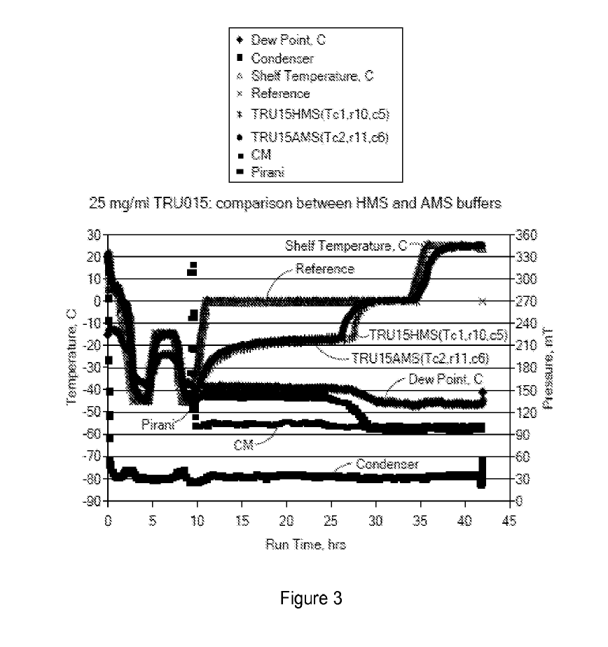

In this example, two formulations were developed, an AMS and an HMS formulation. Acetate-Mannitol-Sucrose (AMS) based formulation contains 20 mM sodium acetate as a buffer, 4% mannitol as a bulking agent and 1% sucrose as stabilizer. In Histidine-Mannitol-Sucrose (HMS) formulation, 20 mM of histidine was used instead of sodium acetate buffer. The remaining components were the same (e.g., 4% mannitol, 1% sucrose). Solution pH was 6.0 for both formulations. Isotonicity of both formulations was 270 mOsm/kg. Filling volume was 4 ml in 10-ml vials for both formulations giving 100 mg protein per vial. An annealing step at −15° C. was used in the lyophilization process. Without wishing to be bound by any theories, it is contemplated that this annealing step promotes mannitol crystallization. Once mannitol is crystallized, glass transition temperature of the remaining amorphous phase may increase from −35° C. to approximately −23° C. for both AMS and HMS formulations. Structural collapse during lyophilization was not detected up to −16° C. (measured for AMS formulation). Higher glass transition and collapse temperatures, as compared to those in Example 1, allow performing lyophilization cycle at higher shelf temperature significantly decreasing the length of the cycle. Exemplary lyophilization program and exemplary cycle traces are shown in Table 3 and FIG. 3.

TABLE 3
Exemplary lyophilization program for 25
mg TRU-015 in AMS and HMS formulations

| | | | |
|---|---|---|---|
| Step | | | Total cycle |
| # | Step description | Pressure, mT | time, hrs |

Freezing

| | | | |
|---|---|---|---|
| 1 | Hold at 5° C. for 60 min | Atmosphere | 1.25 |
| 2 | Ramp from 5° C. to −45° C. | Atmosphere | 2.92 |
| | in 100 min | | |
| 2 | Hold at −45° C. for 60 min | Atmosphere | 3.92 |
| 3 | Ramp from −45° C. to −15° C. | Atmosphere | 4.92 |
| | in 60 min | | |
| 4 | Hold at −15° C. for 120 min | Atmosphere | 6.92 |
| 5 | Ramp from −15° C. to −45° C. | Atmosphere | 7.92 |
| | in 60 min | | |
| 6 | Hold at −45° C. for 30 min | Atmosphere | 8.42 |
| 7 | Vacuum initiating | 100 | 8.75 |

Primary drying

| | | | |
|---|---|---|---|
| 8 | Ramp from −45° C. to 0° C. | 100 | 10.25 |
| | in 90 min | | |
| 9 | Hold at 0° C. for 1380 min | 100 | 33.25 |

Secondary drying

| | | | |
|---|---|---|---|
| 10 | Ramp from 0° C. to 25° C. | 100 | 34.92 |
| | in 100 min | | |
| 11 | Hold at 25° C. for 360 min | 100 | 40.92 |

Data show that primary drying was performed at product temperatures below collapse temperature (e.g., ≦−16° C.). Primary drying was completed before the secondary drying ramp as indicated by Pirani, Dew point sensor and product thermocouples. Cake appearance was acceptable for both formulations. Sub-ambient DSC showed less mannitol crystallinity in HMS buffer as compared to AMS buffer. Protein degradation due to lyophilization was similar for both formulations (0.3% HMW in AMS versus 0.5% HMW in HMS based formulation).

### Example 3

### 50 mg/ml TRU-015 in HMS Formulation

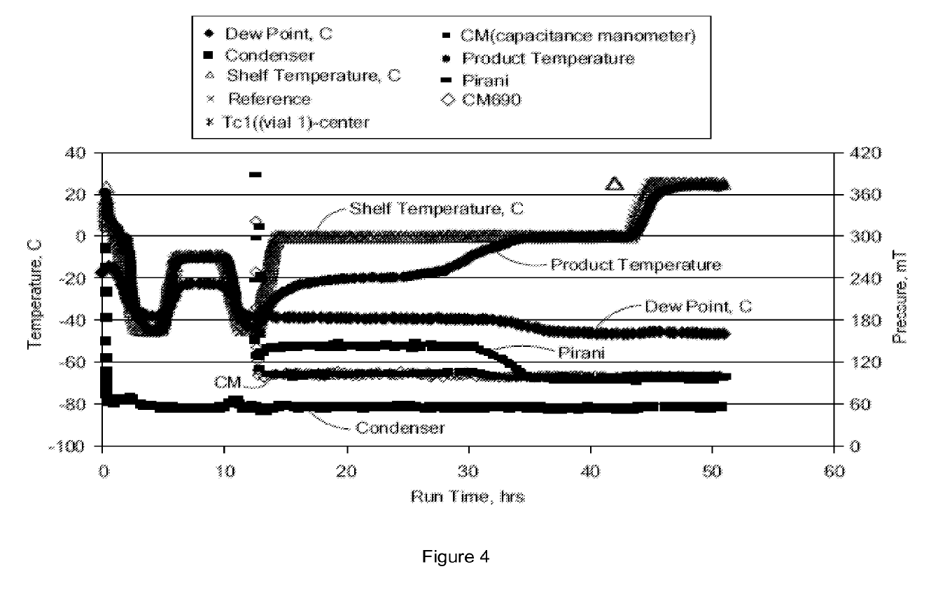

In this example, the concentration of TRU-015 was increased from 25 mg/ml to 50 mg/ml in formulations. Therefore, at a 4.3-ml fill volume in a 10 ml vial, protein content in a vial increased to a calculated value of 215 mg/vial. HMS formulation was employed for the 50-mg/ml-dosage form. The HMS formulation used in this example contained 20 mM histidine as a buffer, 4% mannitol as a bulking agent and 1% sucrose as a stabilizer. The formulation was at pH 6.0. Onset of mannitol crystallization, measured by DSC, was about −23° C. Annealing temperature was approximately −10° C. for this formulation. Annealing time was approximately 4 hours. Glass transition temperature of 50 mg/ml TRU-015 in HMS was −9° C. Primary drying was at a shelf temperature of about 0° C. Exemplary cycle program and exemplary cycle traces are shown in Table 4 and FIG. 4.

TABLE 4
Exemplary lyophilization program for
50 mg/ml TRU-015 in HMS formulation

| | | | |
|---|---|---|---|
| Step | | | Total cycle |
| # | Step description | Pressure, mT | time, hrs |

Freezing

| | | | |
|---|---|---|---|
| 1 | Hold at 5° C. for 45 min | Atmosphere | 1.00 |
| 2 | Ramp to −45° C. in 100 min | Atmosphere | 2.67 |
| 3 | Hold at −45° for 90 min | Atmosphere | 4.17 |
| 4 | Ramp to −10° C. in 70 min | Atmosphere | 5.33 |
| 5 | Hold at −10° C. for 240 min | Atmosphere | 9.33 |
| 6 | Ramp to −45° C. in 70 min | Atmosphere | 10.50 |
| 7 | Hold at −45° C. for 30 min | Atmosphere | 11.00 |
| 8 | Vacuum initiating | 100 | 11.50 |

Primary drying

| | | | |
|---|---|---|---|
| 9 | Ramp to 0° C. in 90 min | 100 | 13.00 |
| 10 | Hold at 0° C. for 1740 min | 100 | 42 |

Secondary drying

| | | | |
|---|---|---|---|
| 11 | Ramp from 0° C. to 25° C. | 100 | 43.67 |
| | in 100 min | | |
| 12 | Hold at 25° C. for 360 min | 100 | 49.67 |

The product temperature during primary drying was below the glass transition temperature. Primary drying was completed prior to secondary drying as indicated by Pirani, Dew point sensor and thermocouples. Cake appearance was acceptable with residual moisture as low as 0.5%. Glass transition temperature of dry powder was above 100° C. Incomplete mannitol crystallization was observed. A small amount of amorphous mannitol was seen crystallizing at onset temperature of approximately 45° C. This still allows accelerated storage at temperatures up to 40° C. Reconstitution time was approximately 2 minutes. Polysorbate-80 may be added to lyophilized solution or to diluent for reconstitution. Increase in fill volume from 4 ml to 4.3 ml allowed delivery of at least 200 mg of TRU-015 from a single vial at protein concentrations above 48 mg/ml. Exemplary percentage of HMW species upon storage was summarized in Table 5.

TABLE 5
Stability of lyophilized TRU-015 in HMS buffer

| | HMW, % | | | | | | | |
|---|---|---|---|---|---|---|---|---|
| Storage | | 1.5 | 3 | 4.5 | 6 | 12 | 18 | 24 |
| temperature | T₀ | month | months | months | months | months | months | months |
| 4° C. | 1.7 | 2.4 | 2.4 | 2.1 | 2.4 | 2.7 | 2.6 | 2.7 |
| 25° C. | | 3.1 | 3.4 | 3.2 | 3.8 | 4.7 | 5.1 | 5.6 |
| 40° C. | | 4.8 | 5.8 | 6.5 | 7.6 | 10.8 | — | — |

Analysis of stability trends show that 50 mg/ml TRU-015 in HMS buffer is predicted to be stable for 2 years at 4° C.

### Example 4

### 100 mg/ml TRU-015 in HMS Formulation

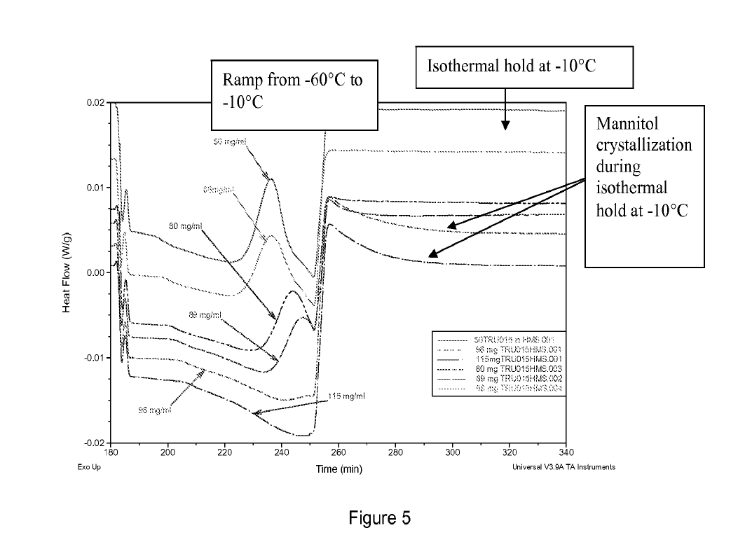

In this example, a formulation was developed suitable for the subcutaneous dosage form (“SQ”), which is typically a valuable option in commercialization of a new drug. Due to a restriction on injection volume (e.g., 1.0 ml), the concentration of protein typically should be at least 100 mg/ml. Another restriction is the isotonicity of buffer, which typically should be in the range between 260 and 320 mOsm/kg. Thus, in this experiment, a formulation for a protein concentration of at least 100 mg/ml was developed. Specifically, an HMS buffer (20 mM histidine, 4% mannitol, 1% sucrose, pH=6.0) with calculated isotonicity value of 270 mOsm/kg was used in this formulation. DSC shows the possible mannitol crystallization in HMS formulation up to 115 mg of protein per ml (FIG. 5).

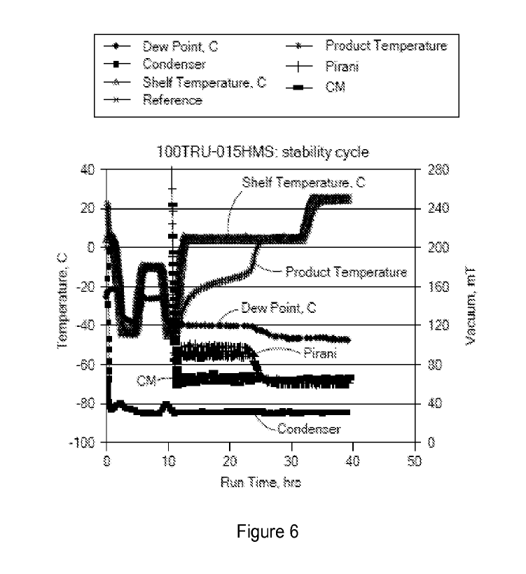

Without wishing to be bound by any theories, it is contemplated that crystalline mannitol is not only a good bulking agent/cake former, but also helps in reconstitution of high concentration proteins. Typically, formulations containing crystalline mannitol dissolved much faster as opposed to amorphous protein-sucrose-mannitol mixtures. Therefore, the evidence of mannitol crystallization at protein concentration of 100 mg/ml indicates that the HMS-based formulation may be particularly suitable for lyophilizing TRU-015 at high concentrations (e.g., 50 mg/ml to 150 mg/ml). DSC also shows that after crystallization of mannitol at −10° C., the glass transition temperature increased to −9° C. allowing aggressive primary drying at the shelf temperature of 5° C. Exemplary lyophilization program and exemplary cycle are shown in Table 6 and FIG. 6 respectively.

TABLE 6
Exemplary lyophilization program for
100 mg/ml TRU-015 in HMS formulation

| | | | |
|---|---|---|---|
| Step | | | Total cycle |
| # | Step description | Pressure, mT | time, hrs |

Freezing

| | | | |
|---|---|---|---|
| 1 | Hold at 5° C. for 45 min | Atmosphere | 1.00 |
| 2 | Ramp to −45° C. in 100 min | Atmosphere | 2.67 |
| 3 | Hold at −45° C. for 120 min | Atmosphere | 4.67 |
| 4 | Ramp to −10° C. in 70 min | Atmosphere | 5.83 |
| 5 | Hold at −10° C. for 180 min | Atmosphere | 8.33 |
| 6 | Ramp to −45° C. in 70 min | Atmosphere | 10.00 |
| 7 | Hold at −45° C. for 30 min | Atmosphere | 10.50 |
| 8 | Vacuum initiating | 75 | 11.00 |

Primary drying

| | | | |
|---|---|---|---|
| 9 | Ramp to 5° C. in 100 min | 75 | 12.67 |
| 10 | Hold at 0° C. for 1740 min | 75 | 31.67 |

Secondary drying

| | | | |
|---|---|---|---|
| 11 | Ramp from 5° C. to 25° C. | 75 | 33.33 |
| | in 100 min | | |
| 12 | Hold at 25° C. for 360 min | 75 | 39.33 |

An annealing step at −10° C. was performed. The time of the annealing step may be 3 to 7 hours. Cake appearance was acceptable with residual moisture of 0.5%. Addition of 0.01% surfactant Polysorbate-80 to the solution before lyophilization allowed reconstitution within 70 sec. The solution became clear within one minute from the moment when reconstitution ends. To account for the vial hold up volume, fill volume in the vial was increased to 1.2 ml. XRD shows that some amorphous mannitol remained in 100 mg/ml TRU-015 in HMS after lyophilization.

Thus, a particularly useful formulation based on this experiment includes 4% mannitol, 1% sucrose, 20 mM histidine, 0.01% Polysorbate-80, 100 mg/ml TRU-015 at pH 6.0 (“HMST” formulation). Exemplary stability data from this formulation during storage is shown in Table 7.

TABLE 7
Exemplary stability data of 100 mg/ml TRU-015 in HMST buffer

| Storage | HMW, % | | | | |
|---|---|---|---|---|---|
| temperature | T₀ | 1 month | 4 months | 6 months | 12 months |
| 4° C. | 3.6 | 4.4 | 4.5 | 4.6 | 4.8 |
| 25° C. | | 6.0 | 7.1 | 7.5 | 9.1 |
| 40° C. | | 9.8 | 13.7 | 15.2 | 20.4 |

### Example 5

### Subcutaneous Formulations Containing TRU-015 at 100 mg/ml

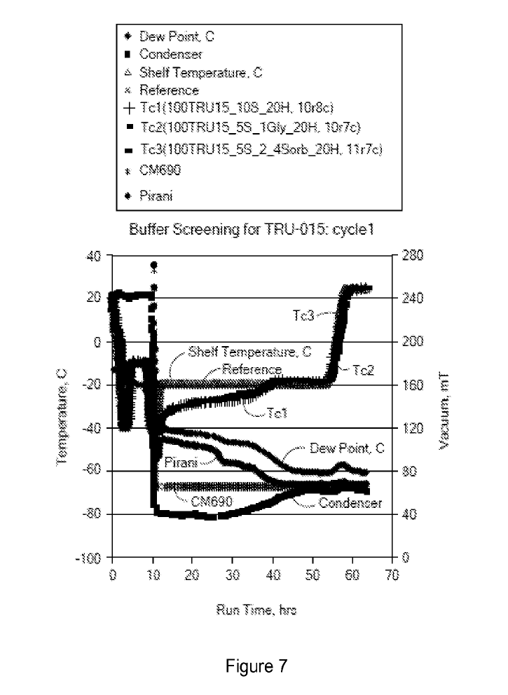

To further improve stability of lyophilized TRU-015, the amount of amorphous stabilizer can be increased while maintaining isotonicity of buffer. It was contemplated that a mass ratio of stabilizer to protein of approximately 1:1 can improve stability at room temperature storage. Thus, the histidine-based formulation used in this experiment included protein at a concentration of 100 mg/ml, sucrose at a concentration of 100 mg/ml (10%), and histidine at a concentration of 20 mM. Isotonicity of this formulation was calculated to be about 312 mOsm/kg. Glass transition temperature of this formulation was approximately −25° C. This 10% sucrose based formulation had a viscosity of (3.9 cPs) compared to HMS formulation (20 mM histidine, 4% mannitol, 1% sucrose, pH 6.0), for which viscosity was determined to be 2.3 cPs. Two alternative formulations were developed, one containing glycine and the other containing sorbitol as stabilizers and isotonicity agents. To decrease viscosity, the concentration of sucrose was decreased from 10% to 5%. To maintain isotonicity of the buffer, the concentration of glycine was about 1% giving 299 mOsm/kg calculated isotonicity in a final formulation. Alternatively, the concentration of sorbitol was about 2.4% giving 298 mOsm/kg calculated isotonicity in a final formulation. The viscosity of the glycine-containing formulation was about 2.7 cPs and the viscosity of the sorbitol-containing formulation was about 3.4 cPs. The glass transition of the glycine-containing formulation was approximately −21° C., and the glass transition of the sorbitol-containing formulation was about −22.5° C. One lyophilization cycle was designed for all three formulations in this example to provide sufficient drying process below the glass transition temperatures. Exemplary lyophilization program for the formulations and exemplary cycle traces are shown in Table 8 and FIG. 7 respectively.

TABLE 8
Exemplary lyophilization program for 100 mg/ml TRU-015

| | | | |
|---|---|---|---|
| Step | | | Total cycle |
| # | Step description | Pressure, mT | time, hrs |

Freezing

| | | | |
|---|---|---|---|
| 1 | Hold at 5° C. for 45 min | Atmosphere | 1.00 |
| 2 | Ramp to −40° C. in 90 min | Atmosphere | 2.50 |
| 3 | Hold at −40° for 60 min | Atmosphere | 3.50 |
| 4 | Ramp to −10 in 60 min | Atmosphere | 4.50 |
| 5 | Hold at −10° C. for 240 min | Atmosphere | 8.50 |
| 6 | Ramp to −40° C. in 60 min | Atmosphere | 9.50 |
| 7 | Hold at −40° C. for 30 min | Atmosphere | 10.00 |
| 8 | Vacuum initiating | 65 | 10.50 |

Primary drying

| | | | |
|---|---|---|---|
| 9 | Ramp to −20° C. in 40 min | 65 | 11.17 |
| 10 | Hold at −20° C. for 2520 min | 65 | 53.17 |

Secondary drying

| | | | |
|---|---|---|---|
| 11 | Ramp from −20° C. to 25° C. | 65 | 56.92 |
| | in 225 min | | |
| 12 | Hold at 25° C. for 360 min | 65 | 62.92 |

This cycle provided acceptable cake appearance for all three formulations. Residual moisture was low; glass transition temperatures were high, allowing high temperature storage during accelerated stability study. The characteristics of exemplary lyophilized TRU-015 formulations are summarized in Table 9.

TABLE 9
Characteristics of exemplary lyophilized powder
of 100 mg/ml TRU-015 SQ formulations. All formulations
contain 20 mM histidine as a buffer.

| | | | Reconstitution time, s | |
|---|---|---|---|---|
| | | Glass | Without | With 0.01% |
| | Residual | transition, | Poly- | Poly- |
| Formulation | moisture, % | ° C. | sorbate-80 | sorbate-80 |
| 10% sucrose | 0.37 ± 0.01 | 85.9 | 180 | 179 |
| (SH formulation) | | | | |
| 5% sucrose + 1% | 0.18 ± 0.06 | 61.3 | 560 | 548 |
| glycine | | and | | |
| (SGH | | 97.5 | | |
| formulation) | | | | |
| 5% sucrose + | 0.17 ± 0.01 | 70.5 | 551 | 735 |
| 2.4% sorbitol | | | | |
| (SSH | | | | |
| formulation) | | | | |

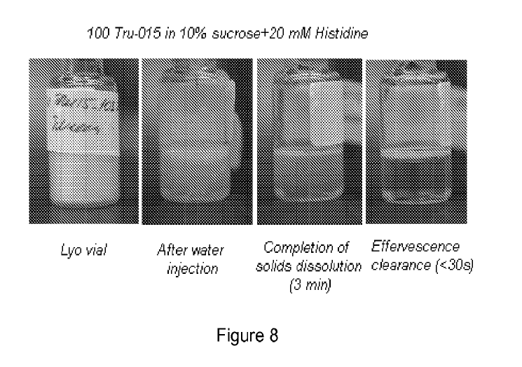

Reconstitution of 100 mg/ml TRU-015 in 10% sucrose-20 mM histidine formulation is shown in FIG. 8. Total reconstitution time from the beginning of water injection to the moment when solution has been cleared from effervescence was not more than 5 minutes.

The effect of Polysorbate-80 (“Tween”) on reconstitution of SQ solution was also studied. Three SQ (e.g., 100 mg/ml protein concentration) formulations were co-lyophilized with 0.01% Tween and without Tween.

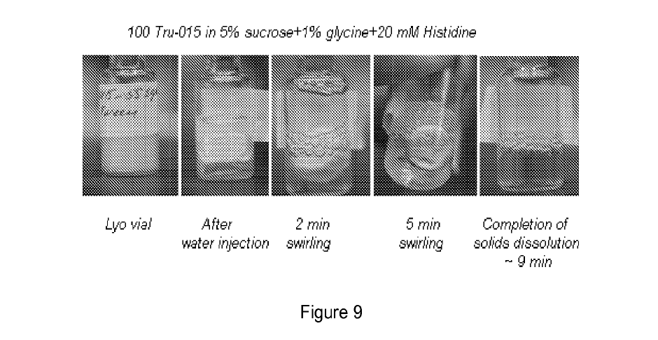

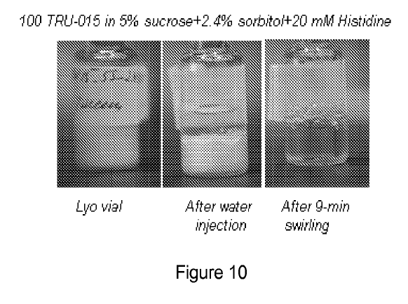

Polysorbate-80 aided in clearing solutions from effervescence after reconstitution. A 10% sucrose based formulation demonstrated reasonable reconstitution time compared to glycine and sorbitol containing formulations. FIGS. 8-10 show exemplary data illustrating the effect different sucrose concentrations may have on reconstituting the lyophilized product with protein at a concentration of 100 mg/ml. Exemplary stability data of SQ formulations are shown in Table 10.

TABLE 10
Exemplary stability data of TRU-015 in three SQ formulations

| | | HMW, % | | | | | |
|---|---|---|---|---|---|---|---|
| | Storage | | 1.5 | 3 | 6 | 9 | 12 |
| Formulations | temperature | T₀ | months | months | months | months | months |
| 10% sucrose + | 4° C. | 3.5 | 3.7 | 3.6 | 3.6 | 3.7 | 3.7 |
| 20 mM histidine + | 25° C. | | 3.8 | 3.8 | 4.0 | 4.2 | 4.2 |
| 0.01% Tween 80 | 40° C. | | 4.3 | 4.4 | 4.9 | 5.4 | — |
| 5% sucrose + | 4° C. | 3.3 | 3.4 | 3.3 | 3.5 | — | 3.8 |
| 1% glycine + | 25° C. | | 3.9 | 4.0 | 4.3 | — | 4.9 |
| 20 mM histidine + | 40° C. | | 4.9 | 5.6 | 6.3 | — | — |
| 0.01% Tween 80 | | | | | | | |
| 5% sucrose + 2.4% | 4° C. | 3.1 | 3.4 | 3.2 | 3.4 | — | 3.5 |
| sorbitol + 20 mM | 25° C. | | 3.7 | 3.5 | 3.9 | — | 4.4 |
| histidine + 0.01% | 40° C. | | 4.5 | 4.6 | 5.5 | — | — |
| Tween 80 | | | | | | | |

### Example 6

### TRU-015 Formulations for Subcutaneous Administration at a Protein Concentration of 200 mg/ml

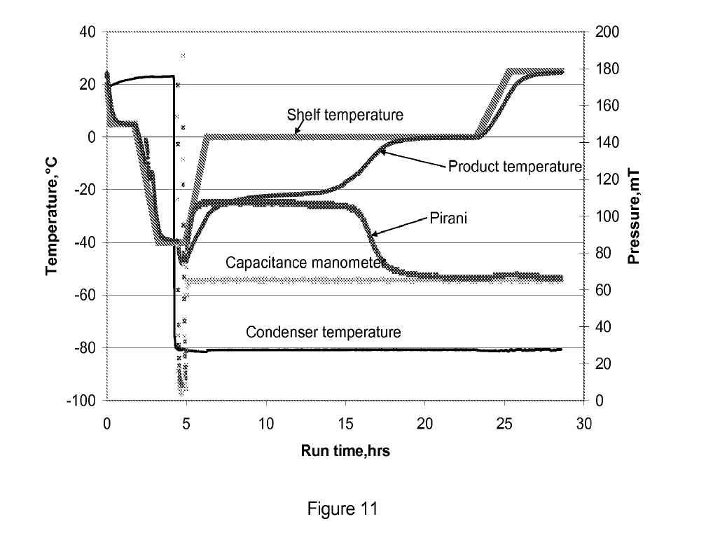

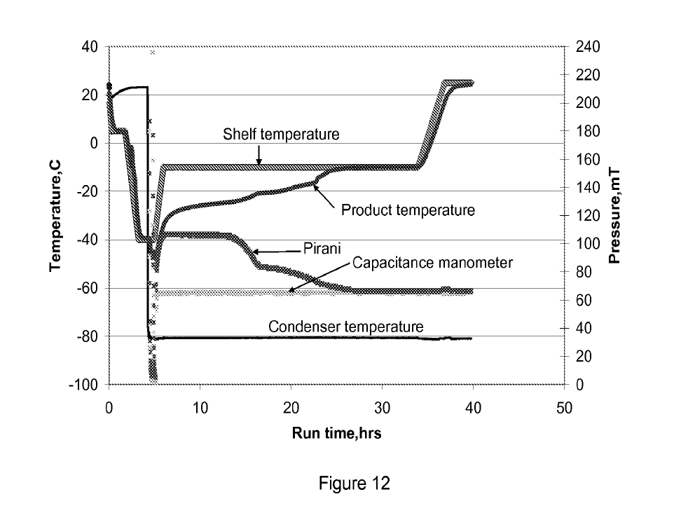

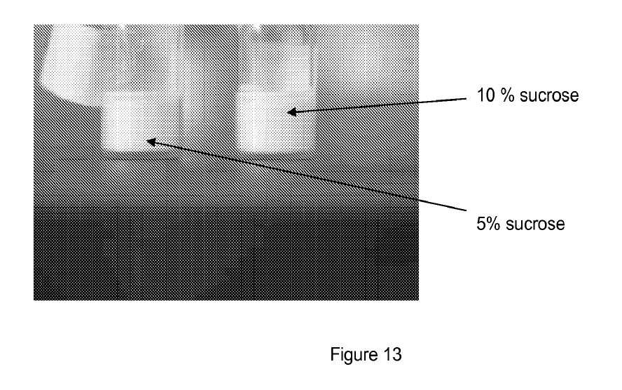

In this experiment, formulations were developed to facilitate delivery of a protein dosage of about 200 mg/vial via subcutaneous administration. Thus, all formulations used in this experiment contained a protein concentration of approximately 200 mg/ml. Exemplary formulations were developed with low (5%) and high (10%) sucrose concentrations as a lyoprotectant (stabilizer). Both formulations contain 10 mM histidine, and 0.01% Polysorbate-80. The low sucrose formulation was predicted to have a lower viscosity. The high sucrose formulation, however, may be more stable at room temperature. The glass transition of the 5% sucrose-based formulation was −21° C. The formulation with 10% sucrose has a glass transition of −26° C. Different lyophilization programs were developed for the two formulations, and exemplary process steps are shown in Tables 11 and 12. Exemplary lyophilization cycles for each process are shown in FIGS. 11 and 12. The cake appearance was acceptable for both formulations and is shown in FIG. 13.

TABLE 11
Exemplary lyophilization program for 200 mg/ml TRU-015
in 5% sucrose, 10 mM histidine, 0.01% Polysorbate 80

| | | | |
|---|---|---|---|
| Step | | | Total cycle |
| # | Step description | Pressure, mT | time, hrs |

Freezing

| | | | |
|---|---|---|---|
| 1 | Ramp 1° C./min to 5° C. | Atmosphere | 0.25 |
| 2 | Hold at 5° C. for 90 min | Atmosphere | 1.75 |
| 3 | Ramp 0.5° C./min to −40° C. | Atmosphere | 3.25 |
| 4 | Hold at −40° for 60 min | Atmosphere | 4.25 |
| 5 | Vacuum initiating | 65 | 4.75 |

Primary drying

| | | | |
|---|---|---|---|
| 6 | Hold at −40° C. for 30 min | 65 | 5.25 |
| 7 | Ramp 0.5° C./min to 0° C. | 65 | 6.58 |
| 8 | Hold at 0° C. for 1020 min | 65 | 23.58 |

Secondary drying

| | | | |
|---|---|---|---|
| 9 | Ramp 0.2° C./min to 25° C. | 65 | 25.67 |
| 10 | Hold at 25° C. for 240 min | 65 | 29.67 |

TABLE 12
Exemplary lyophilization program for 200 mg/ml TRU-015
in 10% sucrose, 10 mM histidine, 0.01% Polysorbate 80

| | | | |
|---|---|---|---|
| Step | | | Total cycle |
| # | Step description | Pressure, mT | time, hrs |

Freezing

| | | | |
|---|---|---|---|
| 1 | Ramp 1° C./min to 5° C. | Atmosphere | 0.25 |
| 2 | Hold at 5° C. for 90 min | Atmosphere | 1.75 |
| 3 | Ramp 0.5° C./min to −40° C. | Atmosphere | 3.25 |
| 4 | Hold at −40° for 60 min | Atmosphere | 4.25 |
| 5 | Vacuum initiating | 65 | 4.75 |

Primary drying

| | | | |
|---|---|---|---|
| 6 | Hold at −40° C. for 30 min | 65 | 5.25 |
| 7 | Ramp 0.5° C./min to −10° C. | 65 | 6.25 |
| 8 | Hold at −10° C. for 1680 min | 65 | 34.25 |

Secondary drying

| | | | |
|---|---|---|---|
| 9 | Ramp 0.2° C./min to 25° C. | 65 | 37.17 |
| 10 | Hold at 25° C. for 240 min | 65 | 41.17 |

TABLE 13
Exemplary stability data for TRU-015
before and after lyophilization.

| | Pre-lyo | HMW, % |
|---|---|---|
| Formulation | HMW,% | T₀ |
| 10% sucrose + 10 mM | 1.1 | 1.9 |
| histidine + 0.01% | | |
| Polysorbate 80 | | |
| 5% sucrose + 10 mM | 1.1 | 2.0 |
| histidine + 0.01% | | |
| Polysorbate 80 | | |

Data from this experiment shows that TRU-015 can withstand lyophilization stresses using formulations described herein even at a protein concentration as high as 200 mg/ml.

### Example 7

### Lyophilization Process for SBI-087 Formulation

In this example, a formulation was designed for the lyophilization of SBI-087 at a concentration of 50 mg/ml. The formulation contains 5% sucrose, 10 mM methionine, 10 mM histidine and 0.01% polysorbate 80 at pH 6.0. An exemplary lyophilization program is shown in Table 14.

TABLE 14
Exemplary lyophilization program for SBI-087 (baseline cycle).

| | | | |
|---|---|---|---|
| Step | | | Total cycle |
| # | Step description | Pressure, mT | time, hrs |

Freezing

| | | | |
|---|---|---|---|
| 1 | Ramp to 5° C. in 15 min | Atmosphere | 0.25 |
| 2 | Hold at 5° C. for 60 min | Atmosphere | 1.25 |
| 3 | Ramp from 5° C. to −40° C. | Atmosphere | 2.75 |
| | in 90 min | | |
| 4 | Hold at −40° C. for 240 min | Atmosphere | 6.75 |
| 5 | Vacuum initiating | 100 | 7.15 |

Primary drying

| | | | |
|---|---|---|---|
| 6 | Ramp from −40° C. to 0° C. | 100 | 8.48 |
| | in 80 min | | |
| 7 | Hold at 0° C. for 1500 min | 100 | 33.48 |

Secondary drying

| | | | |
|---|---|---|---|
| 8 | Ramp from 0° C. to 25° C. | 100 | 35.57 |
| | in 125 min | | |
| 9 | Hold at 25° C. for 240 min | 100 | 39.57 |

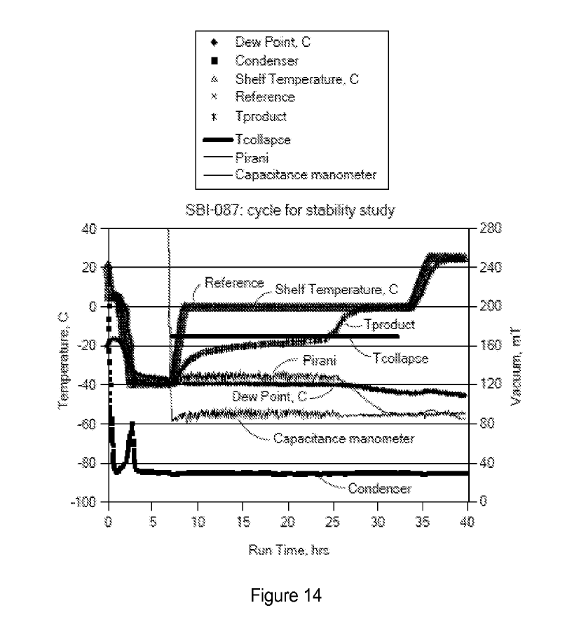

Exemplary process parameters and experimental data are also shown in FIG. 14.

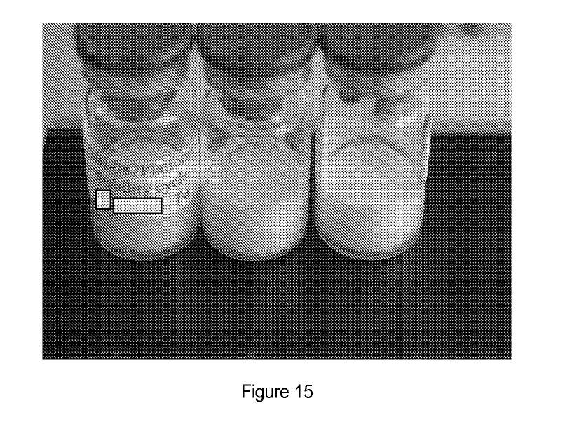

As can be seen in FIG. 14, the product temperature during lyophilization in this case did not exceed the collapse temperature of −15° C. for the SBI-087 formulation. Some cake shrinkage was observed after lyophilization (FIG. 15). However, the cake appearance of lyophilized material was acceptable.

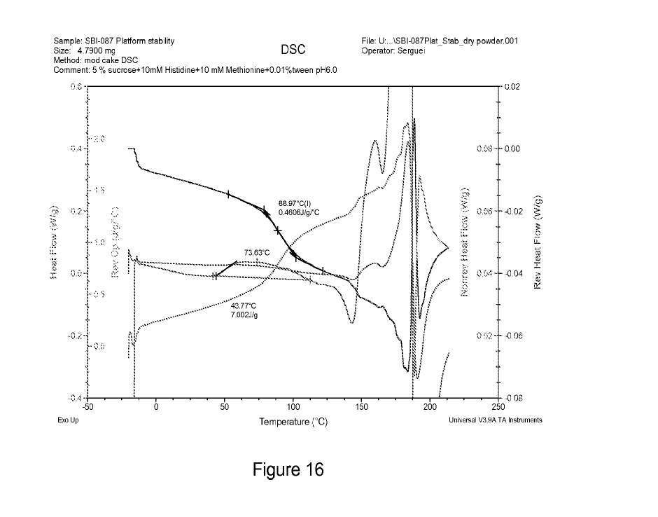

The residual moisture of lyophilized material was 0.37±0.01%. An exemplary Differential Scanning calorimeter (“DSC”) scan is shown in FIG. 16.

The onset of exothermic event occurred at approximately 44° C. The glass transition temperature was approximately 89° C. Based on the lyophilized product properties, and even considering some moisture transfer to the material during storage, it is expected that the lyophilized product can be stored at room temperature without phase transitions.

### Example 8

### Effect of Polysorbate 80 on Reconstitution of SBI-087

In this example a surfactant, Polysorbate 80, was added to the formulation to evaluate its effect on reconstitution. The reconstituted solution of SBI-087 cleared within 1 minute after solids dissolved in solution that contained polysorbate 80. The solution in vials without surfactant remained turbid for at least 1 hour. No difference in protein quality between materials with and without polysorbate 80 was detected. Therefore, without wishing to be bound by any theories, it was contemplated that the “opalescence” was attributed to the air bubbles that were quickly dissipated in the presence of polysorbate.

### Example 9

### Effect of Methionine on Stability of SBI-087

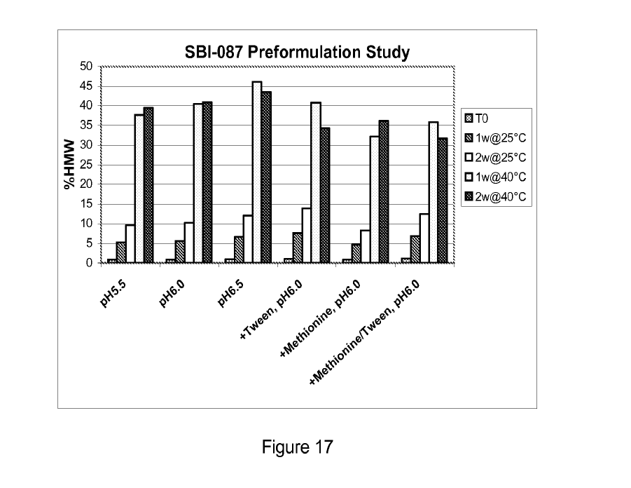

A liquid stability study was performed to confirm the appropriate pH and excipient at elevated temperature. The base formulation is 10 mM histidine, 5% sucrose. The effect of pH (ranging from 5.5 to 6.5), and addition of 0.01% polysorbate 80 and 10 mM Methionine on high molecular weight species (“HMW”) formation were tested. As shown in FIG. 17, an optimum pH for SBI-087 may be in the range of pH 5.5-6.0. In addition, methionine may be beneficial for reducing HMW formation.

### Example 10

### Robustness Study for SBI-087

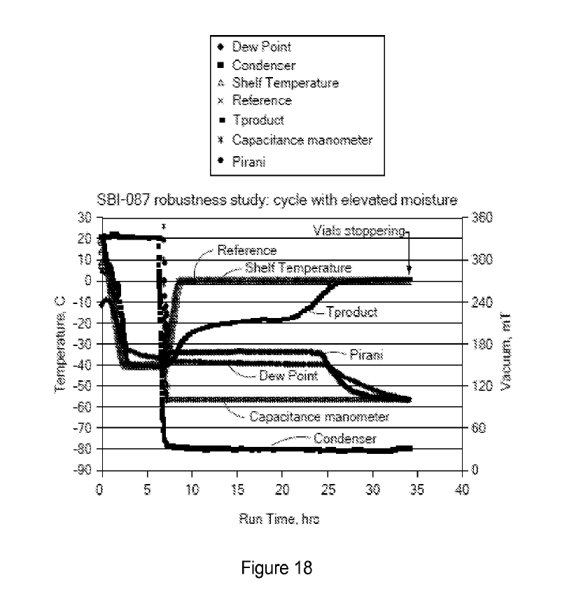

To assess the robustness of the formulation to cycle deviations, additional studies were performed on SBI-087 in the formulation as described in Example 7 (i.e., 5% sucrose, 10 mM histidine, 10 mM methionine and 0.01% polysorbate 80, and 50 mg/ml protein concentration at a pH of 6.0). Due to unpredicted process deviations, the residual moisture in the lyophilized material could potentially increase to a level above the normal average moisture level. Therefore, a suitable formulation should provide enough “resistance” to the increase in mobility due to moisture increase. In order to show that this formulation provides sufficient stability, the lyophilization cycle of Table 14 was performed with one exception: at the end of primary drying, vials were stoppered in order to leave the lyophilized samples with the higher than normal moisture content. An exemplary lyophilization cycle is shown in FIG. 18. The cake appearance of lyophilized material was similar to that in FIG. 15.

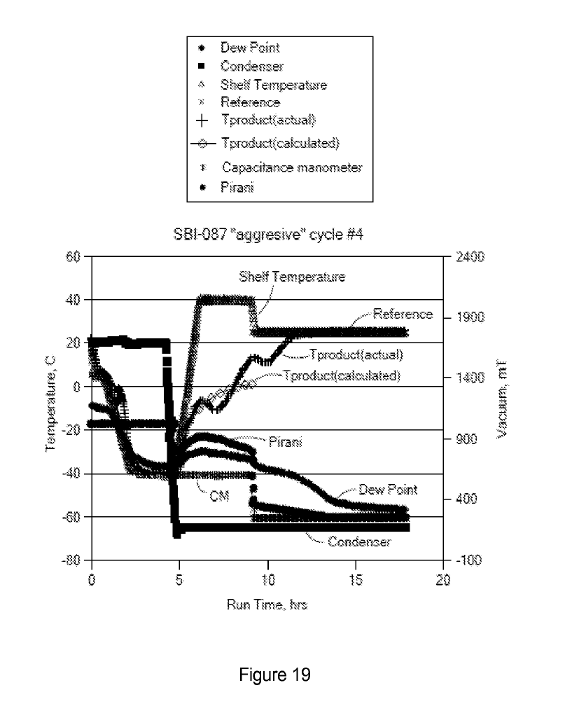

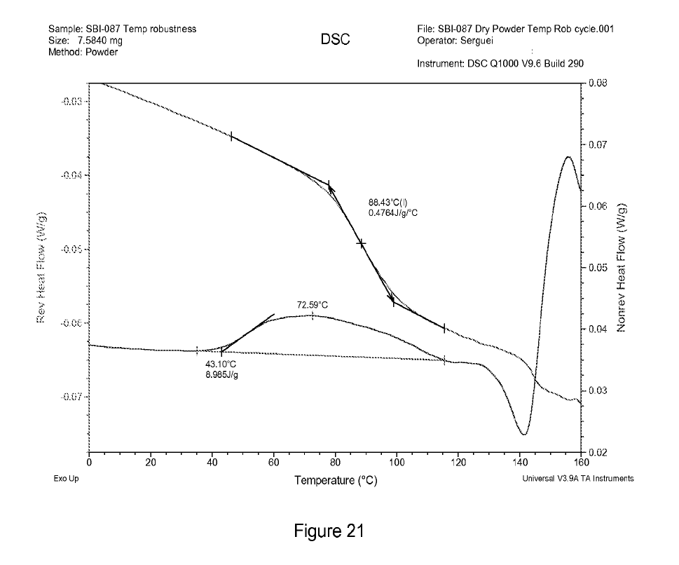

It is not unusual to experience pressure and shelf temperature deviations during commercial lyophilization. Those deviations, always unpredictable, could result in a product temperature increase to the collapse temperature or even exceed it. To test for these process deviations, several “aggressive” cycles were performed at elevated shelf temperature and pressure during primary drying. The design of these cycles was to reach and exceed the collapse temperature during primary drying and assess the resulting product quality. Exemplary lyophilization cycle parameters are shown in Table 15. An example of aggressive cycle (cycle if 4) is also shown in FIG. 19. An example of DSC for SBI-087 dry powder is shown in FIG. 21

TABLE 15
Exemplary process parameters for the “aggressive” lyophilization cycles.

| | Process parameters | | Product | Collapse | |
|---|---|---|---|---|---|
| Cycle | Shelf | | temperature, | temperature, | Visual |
| # | temperature, ° C. | Pressure, mT | ° C. | ° C. | observations |
| 1 | 25 | 100 | −10 | −15 | Slight |
| | | | | | collapse at the |
| | | | | | bottom of vial |
| 2 | 25 | 250 | Between −7 | −15 | Notable |
| | | | and −11 | | collapse at the |
| | | | | | bottom of vial |
| 3 | 25 | 600 mT for 210 min | Between −9 | −15 | Notable |
| | | 500 mT for 30 min | and −12 | | collapse at the |
| | | 450 mT for 30 min | | | bottom of |
| | | 400 mT for 30 min | | | vials |
| | | 350 mT for 30 min | | | |
| | | 300 mT for 30 min | | | |
| | | 250 mT for 240 min | | | |
| 4 | 40 | 600 mT | Between −6 | −15 | Severe |
| | | | and −11 | | collapse at the |
| | | | | | bottom of |
| | | | | | vials (~half |
| | | | | | of cake) |

Note:
1. Freezing step was the same as shown in Table 14.
2. Product temperature is the value of temperature before the thermocouple has lost contact with the ice.

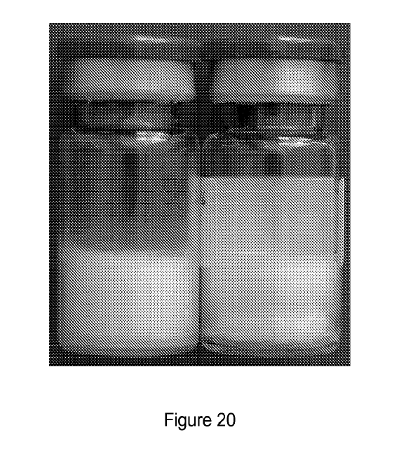

As shown in FIG. 19, the product temperature quickly increased above the collapse temperature to approximately −6° C. and then dropped to the minimum value of −11° C. Calculated product temperature profile indicates that product temperature could potentially exceed the melting point of ice. The collapse of cake structure resulted in loss of contact between the material and the bottom of the vial. Therefore, the heat flux from the bottom of the vial to the product is likely to be reduced, shown as a temperature dip during primary drying. The evidence of collapse from this example can be seen in FIG. 20.

Exemplary residual moisture values and exemplary thermal characteristics of SBI-087 dry powder samples from the robustness cycles are shown in Table 16.

TABLE 16
Exemplary residual moisture and DSC data for SBI-087: comparison
between lyophilization cycles (N/A—not available)

| | Residual moisture, | Tg, | Onset of exothermic |
|---|---|---|---|
| Cycle | % | ° C. | event, ° C. |
| Elevated moisture | 1.25 ± 0.09 | 70.6 | 41.8 |
| cycle (FIG. 18) | | | |
| “Aggressive” | 0.36 ± 0.01 | 88.4 | 43.9 |
| cycle #1 | | | |
| “Aggressive” | 0.62 ± 0.03 | N/A | N/A |
| cycle #2 | | | |
| “Aggressive” | 0.66 ± 0.08 | N/A | N/A |
| cycle #3 | | | |
| “Aggressive” | 0.76 ± 0.02 | N/A | N/A |
| cycle #4 | | | |

NA—not available

An increase in moisture con tent during the elevated moisture cycle resulted in an 18-degree decrease in glass transition temperature. The onset of exothermic event also decreased. However, all glass transitions for examined materials are still higher than storage temperature indicating a low mobility in the amorphous phase. Glass transition temperatures of materials from “aggressive” cycles 2-4 are expected to be within the range 71° C. to 88° C. based on moisture data. Furthermore, based on moisture and DSC data, it is predicted that examined process deviations should not notably affect the rate of degradation during storage at 4° C. Exemplary stability data support this prediction are shown in Table 17.

TABLE 17
Exemplary Stability of lyophilized SBI-087 material
manufactured using different cycles

| | | HMW % | | | | | | | | |
|---|---|---|---|---|---|---|---|---|---|---|
| Lyophilization | Storage | | 1.5 | 3 | 4.5 | 6 | 9 | 12 | 18 | 24 |
| cycle | temperature | T₀ | mo. | mo. | mo. | mo. | mo. | mo. | mo. | mo. |
| Baseline | 4° C. | 2.8 | 2.8 | 2.5 | 2.8 | 3.3 | 2.8 | 2.8 | 2.2 | 3.0 |
| cycle | 25° C. | | 2.8 | 2.7 | 2.9 | 3.6 | 3.2 | 3.2 | 2.7 | 3.6 |
| (Table 1) | 40° C. | | 3.2 | 3.3 | 3.6 | 4.4 | 4.4 | 4.4 | 4.6 | 5.6 |
| Elevated | 4° C. | 1.3 | 1.3 | 1.3 | | | | | | |
| moisture | 25° C. | | 1.3 | 1.4 | | | | | | |
| cycle | 40° C. | | 1.5 | 1.6 | | | | | | |
| (FIG. 5) | | | | | | | | | | |
| “Aggressive” | 4° C. | 1.5 | 1.5 | 1.4 | | | | | | |
| cycle #1 | 25° C. | | 1.6 | 1.6 | | | | | | |
| (Table 2) | 40° C. | | 1.8 | 2.0 | | | | | | |
| “Aggressive” | 4° C. | 3.2 | | | 2.8 | | 2.8 | | | |
| cycle #4 | 25° C. | | | | 2.9 | | 2.9 | | | |
| | 40° C. | | | | 3.4 | | 3.4 | | | |

### Example 11

### Kits with Pre-Filled Diluent Syringe

In this example, kits containing lyophilized SMIP™ protein product and pre-filled diluent syringe are developed for the convenience of reconstitution and administration. A kit with pre-filled diluent syringe typically includes a vial with lyophilized protein, a pre-filled diluent syringe containing reconstitution buffer sterile water for injection, a vial adapter and a syringe plunger rod. The kit may include an instruction manual for use. A pre-filled diluent syringe kit may be used according to the following steps.

First, the vials of lyophilized SMIP™ proteins and the pre-filled diluent syringe are allowed to reach room temperature. Then the plastic flip-top cap from the vial containing the lyophilized protein is removed to expose the central portions of the rubber stopper. The top of the vial is wiped with an antiseptic swab or cloth. After cleaning, the rubber stopper should not be contacted with any surface or person to minimize the chances of contamination. Care should be taken throughout the procedure to minimize the risk of contamination.

Next, the cover from the plastic vial adapter package is removed by peeling it back. Then the vial adapter is placed over the vial and pressed until the adapter spike in the adapter penetrates the vial stopper. Next, the plunger rod is threaded to the diluent syringe plunger, patients or physicians should avoid contact with the shaft of the plunger rod while threading the plunger rod to the plunger to minimize the risk of contamination. Next, the plastic, tamper-resistant, tip cap on the diluent syringe is broken off by snapping the perforation in the cap. Contact with the inside of the cap of the syringe tip should be avoided. The cap is then placed on its top on a clean surface in a location where it is unlikely to become contaminated. The cap can be replaced if the reconstituted solution will not be administered immediately.

Next, the packaging of the adapter is lifted away from the adapter and discarded. The vial should be placed on a flat surface. Next the diluent syringe is connected to the vial adapter by threading the tip into the adapter opening until secure. Next, the plunger rod is depressed to inject all of the diluent into the protein vial. Without removing the syringe, the contents of the vial are gently swirled or mixed until the powder is dissolved. The solution is then inspected for any undissolved powder. The solution should then be clear and colorless. Additional vials containing lyophilized SMIP™ protein can be reconstituted in the same manner, if more than one vial is to be administered in one injection.

The vial is then inverted and the solution slowly drawn into the syringe. If more than one vial of SMIP™ protein is to be administered, the syringe should be removed from the vial, leaving the vial adapter attached to the vial without drawing the reconstituted solution into it. A separate large luer lock syringe can be attached and the reconstituted contents drawn into it. This procedure can be repeated for each vial.

The syringe can be detached from the vial adapter by gently pulling and turning the syringe counter-clockwise. The vial is then discarded with the adapter still attached. Typically, the reconstituted SMIP™ protein should be administered within approximately 3 hours when stored at room temperature.

EXEMPLARY SMIP™ SEQUENCES
Italics: Linker sequence
Underline: CDR sequences

| Construct Name | | | |
|---|---|---|---|
| | VK3 | VH5 | |
| 18011 | 2Lm19- | 2H5m3 | EIVLTQSPATLSLSPGERATLSCRASQSVSYIV |
| | 3 | | WYQQKPGQAPRLLIYAPSNLASGIPARFSGS |
| | | | GSGTDFTLTISSLEPEDFAVYYCQQWSFNPPT |
| | | | FGQGTKVEIKDGGGSGGGGSGGGGTGEVQLV |
| | | | QSGAEVKKPGESLKISCKGSGYSFTSYNMHW |
| | | | VRQMPGKGLEWMGAIYPGNGDTSYNQKFKG |
| | | | QVTISADKSISTAYLQWSSLKASDTAMYYCAR |
| | | | SYYSNSYWYFDLWGRGTLVTVSS |
| | | | (SEQ ID NO: 1) |
| 18008 | 2Lm5 | 2H5 | EIVLTQSPATLSLSPGERATLSCRASQSVSYMH |
| | | | WYQQKPGQAPRLLIYAPSNLASGIPARFSGSGS |
| | | | GTDFTLTISSLEPEDFAVYYCQQWSFNPPTFGQ |
| | | | GTKVEIKDGGGSGGGGSGGGGTGEVQLVQSGA |
| | | | EVKKPGESLKISCKGSGYSFTSYNMHWVRQMP |
| | | | GKGLEWMGAIYPGNGDTSYNQKFKGQVTISA |
| | | | DKSISTAYLQWSSLKASDTAMYYCAR |
| | | | VVYYSNSYWYFDLWGRGTLVTVSS |
| | | | (SEQ ID NO: 2) |
| 18010 | 2Lm19- | 2H5 | EIVLTQSPATLSLSPGERATLSCRASQSVSYIV |
| | 3 | | WYQQKPGQAPRLLIYAPSNLASGIPARFSGSG |
| | | | SGTDFTLTISSLEPEDFAVYYCQQWSFNPPTF |
| | | | GQGTKVEIKDGGGSGGGGSGGGGTGEVQLV |
| | | | QSGAEVKKPGESLKISCKGSGYSFTSYNMHW |
| | | | VRQMPGKGLEWMGAIYPGNGDTSYNQKFKG |
| | | | QVTISADKSISTAYLQWSSLKASDTAMYYCAR |
| | | | VVYYSNSYWYFDLWGRGTLVTVSS |
| | | | (SEQ ID NO: 3) |
| 18009 | 2Lm5 | 2H5m3 | EIVLTQSPATLSLSPGERATLSCRASQSVSYIV |
| | | | WYQQKPGQAPRLLIYAPSNLASGIPARFSGS |
| | | | GSGTDFTLTISSLEPEDFAVYYCQQWSFNPPT |
| | | | FGQGTKVEIKDGGGSGGGGSGGGGTGEVQLV |
| | | | QSGAEVKKPGESLKISCKGSGYSFTSYNMHW |
| | | | VRQMPGKGLEWMGAIYPGNGDTSYNQKFKG |
| | | | QVTISADKSISTAYLQWSSLKASDTAMYYCA |
| | | | RVVYYSNSYWYFDLWGRGTLVTVSS |
| | | | (SEQ ID NO: 4) |
| 2Lm5 2H3m3 | 2Lm5 | 2H3m3 | EIVLTQSPATLSLSPGERATLSCRASQSVSSYMH |
| | | | WYQQKPGQAPRLLIYAPSNLASGIPARFSGSGS |
| | | | GTDFTLTISSLEPEDFAVYYCQQWSFNPPTFG |
| | | | QGTKVEIKDGGGSGGGGSGGGGTGEVQLLES |
| | | | GGGLVQPGGSLRLSCAASGFTFSSYNMHWVR |
| | | | QAPGKGLEWVSAIYPGNGDTSYNQKFKGRFT |
| | | | ISRDNSKNTLYLQMNSLRAEDTAVYYCA |
| | | | KSYYSNSYWYFDLWGRGTLVTVSS |
| | | | (SEQ ID NO: 5) |
| | VK3 | VH1 | |
| | 2L | 2Hm | EIVLTQSPATLSLSPGERATLSCRASSSVSSYMHW |
| | | | YQQKPGQAPRLLIYAPSNLASGIPARFSGSGSGTD |
| | | | FTLTISSLEPEDFAVYYCQQWSFNPPTFGQGTKV |
| | | | EIKDGGGSGGGGSGGGGSSQVQLVQSGAEVKKP |
| | | | GASVKVSCKASGYTFTSYNMHWVRQAPGQGLE |
| | | | WMGAIYPGNGDTSYNQKFKGRVTMTRDTSTST |
| | | | VYMELSSLRSEDTAVYYCARSVYYSN.YWYFDL |
| | | | WGRGTLVTVSS |
| | | | (SEQ ID NO: 6) |
| | 2Lm | 2Hm | EIVLTQSPATLSLSPGERATLSCRASSSVSYMIW |
| | | | YQQKPGQAPRLLIYAISNLASGIPARFSGSGSGT |
| | | | DFTLTISSLEPEDFAVYYCQQWISNPPTFGQGTK |
| | | | VEIKDGGGSGGGGSGGGGSSQVQLVQSGAEVK |
| | | | KPGASVKVSCKASGYTFTSYNMHWVRQAPGQ |
| | | | GLEWMGAIYPGNGDTSYNQKFKGRVTMTRDT |
| | | | STSTVYMELSSLRSEDTAVYYCAR |
| | | | SVYYSN.YWYFDLWGRGTLVTVSS |
| | | | (SEQ ID NO: 7) |
| | 2Lm | 2H | EIVLTQSPATLSLSPGERATLSCRASSSVSYMIW |
| | | | YQQKPGQAPRLLIYAISNLASGIPARFSGSGSGT |
| | | | DFTLTISSLEPEDFAVYYCQQWISNPPTFGQGTK |
| | | | VEIKDGGGSGGGGSGGGGSSQVQLVQSGAEVK |
| | | | KPGASVKVSCKASGYTFTSYNMHWVRQAPGQ |
| | | | GLEWMGAIYPGNGDTSYNQKFKGRVTMTRDT |
| | | | STSTVYMELSSLRSEDTAVYYCAR |
| | | | VVYYSNSYWYFDLWGRGTLVTVSS |
| | | | (SEQ ID NO: 8) |
| | 2Lm1 | 2Hm | EIVLTQSPATLSLSPGERATLSCRASQSSVSYMH |
| | | | WYQQKPGQAPRLLIYAPSNLASGIPARFSGSGS |
| | | | GTDFTLTISSLEPEDFAVYYCQQWISNPPTFGQG |
| | | | TKVEIKDGGGSGGGGSGGGGSSQVQLVQSGAE |
| | | | VKKPGASVKVSCKASGYTFTSYNMHWVRQAP |
| | | | GQGLEWMGAIYPGNGDTSYNQKFKGRVTMTRD |
| | | | TSTSTVYMELSSLRSEDTAVYYCAR |
| | | | SVYYSN.YWYFDLWGRGTLVTVSS |
| | | | (SEQ ID NO: 9) |
| | 2Lm1 | 2H | EIVLTQSPATLSLSPGERATLSCRASQSSVSYMH |
| | | | WYQQKPGQAPRLLIYAPSNLASGIPARFSGSGSG |
| | | | TDFTLTISSLEPEDFAVYYCQQWISNPPTFGQGTK |
| | | | VEIKDGGGSGGGGSGGGGSSQVQLVQSGAEVKKP |
| | | | GASVKVSCKASGYTFTSYNMHWVRQAPGQGLEW |
| | | | MGAIYPGNGDTSYNQKFKGRVTMTRDTSTSTVY |
| | | | MELSSLRSEDTAVYYCARVVYYSNSYWYFDLW |
| | | | GRGTLVTVSS |
| | | | (SEQ ID NO: 10) |
| | 2Lm2 | 2Hm | EIVLTQSPATLSLSPGERATLSCRASQSVSYMIW |
| | | | YQQKPGQAPRLLIYAISNLASGIPARFSGSGSGT |
| | | | DFTLTISSLEPEDFAVYYCQQWSFNPPTFGQGTK |
| | | | VEIKDGGGSGGGGSGGGGSSQVQLVQSGAEVK |
| | | | KPGASVKVSCKASGYTFTSYNMHWVRQA |
| | | | PGQGLEWMGAIYPGNGDTSYNQKFKGRV |
| | | | TMTRDTSTSTVYMELSSLRSEDTAVYYCA |
| | | | RSVYYSN.YWYFDLWGRGTLVTVSS |
| | | | (SEQ ID NO: 11) |
| | 2Lm3 | 2Hm | EIVLTQSPATLSLSPGERATLSCRASSSVSYMI |
| | | | WYQQKPGQAPRLLIYAISNLASGIPARFSGSG |
| | | | SGTDFTLTISSLEPEDFAVYYCQQWTSNPPTF |
| | | | GQGTKVEIKDGGGSGGGGSGGGGSSQVQLV |
| | | | QSGAEVKKPGASVKVSCKASGYTFTSYNMH |
| | | | WVRQAPGQGLEWMGAIYPGNGDTSYNQKFKG |
| | | | RVTMTRDTSTSTVYMELSSLRSEDTAVYYCA |
| | | | RSVYYSN.YWYFDLWGRGTLVTVSS |
| | | | (SEQ ID NO: 12) |
| | 2Lm4 | 2Hm | EIVLTQSPATLSLSPGERATLSCRASQSVSSYMH |
| | | | WYQQKPGQAPRLLIYAPSNLASGIPARFSGSGS |
| | | | GTDFTLTISSLEPEDFAVYYCQQWTSNPPTFGQ |
| | | | GTKVEIKDGGGSGGGGSGGGGSSQVQLVQSGA |
| | | | EVKKPGASVKVSCKASGYTFTSYNMHWVRQA |
| | | | PGQGLEWMGAIYPGNGDTSYNQKFKGRVTMT |
| | | | RDTSTSTVYMELSSLRSEDTAVYYCAR |
| | | | SVYYSN.YWYFDLWGRGTLVTVSS |
| | | | (SEQ ID NO: 13) |
| | 2Lm5 | 2Hm | EIVLTQSPATLSLSPGERATLSCRASQSVSYMH |
| | | | WYQQKPGQAPRLLIYAPSNLASGIPARFSGSG |
| | | | SGTDFTLTISSLEPEDFAVYYCQQWSFNPPTFG |
| | | | QGTKVEIKDGGGSGGGGSGGGGSSQVQLVQS |
| | | | GAEVKKPGASVKVSCKASGYTFTSYNMHWV |
| | | | RQAPGQGLEWMGAIYPGNGDTSYNQKFKGRV |
| | | | TMTRDTSTSTVYMELSSLRSEDTAVYYCA |
| | | | RSVYYSN.YWYFDLWGRGTLVTVSS |
| | | | (SEQ ID NO: 14) |
| | 2Lm5-1 | 2Hm3 | EIVLTQSPATLSLSPGERATLSCRASQSVSYMH |
| | | | WYQQKPGQAPRLLIYAPSNLASGIPARFSGSGS |
| | | | GTDFTLTISSLEPEDFAVYYCQQWSFNPPTFGQ |
| | | | GTKVEIKDGGGSGGGGSGGGGSSQVQLVQSGA |
| | | | EVKKPGASVKVSCKASGYTFTSYNMHWVRQA |
| | | | PGQGLEWMGAIYPGNGDTSYNQKFKGRVTMT |
| | | | RDTSTSTVYMELSSLRSEDTAVYYCAR |
| | | | S.YYSNSYWYFDLWGRGTLVTVSS |
| | | | (SEQ ID NO: 15) |
| | 2Lm5-2 | 2Hm4 | EIVLTQSPATLSLSPGERATLSCRASQSVSYMH |
| | | | WYQQKPGQAPRLLIYAPSNLASGIPARFSGSGS |
| | | | GTDFTLTISSLEPEDFAVYYCQQWSFNPPTFGQ |
| | | | GTKVEIKDGGGSGGGGSGGGGSSQVQLVQSGA |
| | | | EVKKPGASVKVSCKASGYTFTSYNMHWVRQA |
| | | | PGQGLEWMGAIYPGNGDTSYNQKFKGRVTMT |
| | | | RDTSTSTVYMELSSLRSEDTAVYYCAR |
| | | | V.YYSNSYWYFDLWGRGTLVTVSS |
| | | | (SEQ ID NO: 16) |
| | 2Lm5-3 | 2Hm5 | EIVLTQSPATLSLSPGERATLSCRASQSVSYMH |
| | | | WYQQKPGQAPRLLIYAPSNLASGIPARFSGSGS |
| | | | GTDFTLTISSLEPEDFAVYYCQQWSFNPPTFGQ |
| | | | GTKVEIKDGGGSGGGGSGGGGSSQVQLVQSGA |
| | | | EVKKPGASVKVSCKASGYTFTSYNMHWVRQA |
| | | | PGQGLEWMGAIYPGNGDTSYNQKFKGRVTMT |
| | | | RDTSTSTVYMELSSLRSEDTAVYYCAR |
| | | | SVYY.NSYWYFDLWGRGTLVTVSS |
| | | | (SEQ ID NO: 17) |
| | 2Lm6 | 2Hm | EIVLTQSPATLSLSPGERATLSCRASQSVSYMH |
| | | | WYQQKPGQAPRLLIYAPSNLASGIPARFSGSG |
| | | | SGTDFTLTISSLEPEDFAVYYCQQWTSNPPTF |
| | | | GQGTKVEIKDGGGSGGGGSGGGGSSQVQLV |
| | | | QSGAEVKKPGASVKVSCKASGYTFTSYNMH |
| | | | WVRQAPGQGLEWMGAIYPGNGDTSYNQKFKG |
| | | | RVTMTRDTSTSTVYMELSSLRSEDTAVYYCAR |
| | | | SVYYSN.YWYFDLWGRGTLVTVSS |
| | | | (SEQ ID NO: 18) |
| | 2Lm6-1 | 2Hm3 | EIVLTQSPATLSLSPGERATLSCRASQSVSYMH |
| | | | WYQQKPGQAPRLLIYAPSNLASGIPARFSGSGS |
| | | | GTDFTLTISSLEPEDFAVYYCQQWTSNPPTFGQ |
| | | | GTKVEIKDGGGSGGGGSGGGGSSQVQLVQSGA |
| | | | EVKKPGASVKVSCKASGYTFTSYNMHWVRQA |
| | | | PGQGLEWMGAIYPGNGDTSYNQKFKGRVTMT |
| | | | RDTSTSTVYMELSSLRSEDTAVYYCAR |
| | | | S.YYSNSYWYFDLWGRGTLVTVSS |
| | | | (SEQ ID NO: 19) |
| | 2Lm6-2 | 2Hm4 | EIVLTQSPATLSLSPGERATLSCRASQSVSYMH |
| | | | WYQQKPGQAPRLLIYAPSNLASGIPARFSGSGS |
| | | | GTDFTLTISSLEPEDFAVYYCQQWTSNPPTFGQ |
| | | | GTKVEIKDGGGSGGGGSGGGGSSQVQLVQSGA |
| | | | EVKKPGASVKVSCKASGYTFTSYNMHWVRQA |
| | | | PGQGLEWMGAIYPGNGDTSYNQKFKGRVTMT |
| | | | RDTSTSTVYMELSSLRSEDTAVYYCAR |
| | | | V.YYSNSYWYFDLWGRGTLVTVSS |
| | | | (SEQ ID NO: 20) |
| | 2Lm6-3 | 2Hm5 | EIVLTQSPATLSLSPGERATLSCRASQSVSYMH |
| | | | WYQQKPGQAPRLLIYAPSNLASGIPARFSGSG |
| | | | SGTDFTLTISSLEPEDFAVYYCQQWTSNPPTFG |
| | | | QGTKVEIKDGGGSGGGGSGGGGSSQVQLVQS |
| | | | GAEVKKPGASVKVSCKASGYTFTSYNMHWV |
| | | | RQAPGQGLEWMGAIYPGNGDTSYNQKFKGR |
| | | | VTMTRDTSTSTVYMELSSLRSEDTAVYYCAR |
| | | | SVYY.NSYWYFDLWGRGTLVTVSS |
| | | | (SEQ ID NO: 21) |
| | 2Lm7 | 2Hm | EIVLTQSPATLSLSPGERATLSCRASSSVSYMH |
| | | | WYQQKPGQAPRLLIYATSNLASGIPARFSGSG |
| | | | SGTDFTLTISSLEPEDFAVYYCQQWTSNPPTFG |
| | | | QGTKVEIKDGGGSGGGGSGGGGSSQVQLVQS |
| | | | GAEVKKPGASVKVSCKASGYTFTSYNMHWV |
| | | | RQAPGQGLEWMGAIYPGNGDTSYNQKFKGR |
| | | | VTMTRDTSTSTVYMELSSLRSEDTAVYYCAR |
| | | | SVYYSN.YWYFDLWGRGTLVTVSS |
| | | | (SEQ ID NO: 22) |
| | 2Lm8 | 2Hm | EIVLTQSPATLSLSPGERATLSCRASSSVSYMI |
| | | | WYQQKPGQAPRLLIYAISNLASGIPARFSGSG |
| | | | SGTDFTLTISSLEPEDFAVYYCQQWISNPYTF |
| | | | GQGTKVEIKDGGGSGGGGSGGGGSSQVQLV |
| | | | QSGAEVKKPGASVKVSCKASGYTFTSYNMH |
| | | | WVRQAPGQGLEWMGAIYPGNGDTSYNQKFKG |
| | | | RVTMTRDTSTSTVYMELSSLRSEDTAVYYCA |
| | | | RSVYYSN.YWYFDLWGRGTLVTVSS |
| | | | (SEQ ID NO: 23) |
| | 2Lm9 | 2Hm | EIVLTQSPATLSLSPGERATLSCRASSSVSYMI |
| | | | WYQQKPGQAPRLLIYAISNLASGIPARFSGSG |
| | | | SGTDFTLTISSLEPEDFAVYYCQQWISNPFTFG |
| | | | QGTKVEIKDGGGSGGGGSGGGGSSQVQLVQS |
| | | | GAEVKKPGASVKVSCKASGYTFTSYNMHWV |
| | | | RQAPGQGLEWMGAIYPGNGDTSYNQKFKGR |
| | | | VTMTRDTSTSTVYMELSSLRSEDTAVYYCAR |
| | | | SVYYSN.YWYFDLWGRGTLVTVSS |
| | | | (SEQ ID NO: 24) |
| | 2Lm10 | 2Hm | EIVLTQSPATLSLSPGERATLSCRASSSVSYMI |
| | | | WYQQKPGQAPRLLIYAISNLASGIPARFSGSG |
| | | | SGTDFTLTISSLEPEDFAVYYCQQWISNPLTFG |
| | | | QGTKVEIKDGGGSGGGGSGGGGSSQVQLVQS |
| | | | GAEVKKPGASVKVSCKASGYTFTSYNMHWV |
| | | | RQAPGQGLEWMGAIYPGNGDTSYNQKFKGR |
| | | | VTMTRDTSTSTVYMELSSLRSEDTAVYYCA |
| | | | RSVYYSN.YWYFDLWGRGTLVTVSS |
| | | | (SEQ ID NO: 25) |
| | 2Lm11 | 2Hm | EIVLTQSPATLSLSPGERATLSCRASSSVSYMI |
| | | | WYQQKPGQAPRLLIYAISNLASGIPARFSGSG |
| | | | SGTDFTLTISSLEPEDFAVYYCQQWISNPITFG |
| | | | QGTKVEIKDGGGSGGGGSGGGGSSQVQLVQS |
| | | | GAEVKKPGASVKVSCKASGYTFTSYNMHWV |
| | | | RQAPGQGLEWMGAIYPGNGDTSYNQKFKGR |
| | | | VTMTRDTSTSTVYMELSSLRSEDTAVYYCAR |
| | | | SVYYSN.YWYFDLWGRGTLVTVSS |
| | | | (SEQ ID NO: 26) |
| | 2Lm12 | 2Hm | EIVLTQSPATLSLSPGERATLSCRASQSVSYMH |
| | | | WYQQKPGQAPRLLIYATSNLASGIPARFSGSG |
| | | | SGTDFTLTISSLEPEDFAVYYCQQWSFNPPTFG |
| | | | QGTKVEIKDGGGSGGGGSGGGGSSQVQLVQS |
| | | | GAEVKKPGASVKVSCKASGYTFTSYNMHWV |
| | | | RQAPGQGLEWMGAIYPGNGDTSYNQKFKGR |
| | | | VTMTRDTSTSTVYMELSSLRSEDTAVYYCAR |
| | | | SVYYSN.YWYFDLWGRGTLVTVSS |
| | | | (SEQ ID NO: 27) |
| | 2Lm13 | 2Hm | EIVLTQSPATLSLSPGERATLSCRASQSVSYMH |
| | | | WYQQKPGQAPRLLIYAPSNLASGIPARFSGSGS |
| | | | GTDFTLTISSLEPEDFAVYYCQQWISNPPTFGQG |
| | | | TKVEIKDGGGSGGGGSGGGGSSQVQLVQSGAE |
| | | | VKKPGASVKVSCKASGYTFTSYNMHWVRQAP |
| | | | GQGLEWMGAIYPGNGDTSYNQKFKGRVTMTR |
| | | | DTSTSTVYMELSSLRSEDTAVYYCAR |
| | | | SVYYSN.YWYFDLWGRGTLVTVSS |
| | | | (SEQ ID NO: 28) |
| | 2Lm14 | 2Hm | EIVLTQSPATLSLSPGERATLSCRASQSVSYMH |
| | | | WYQQKPGQAPRLLIYATSNLASGIPARFSGSGS |
| | | | GTDFTLTISSLEPEDFAVYYCQQWISNPPTFGQ |
| | | | GTKVEIKDGGGSGGGGSGGGGSSQVQLVQSGA |
| | | | EVKKPGASVKVSCKASGYTFTSYNMHWVRQA |
| | | | PGQGLEWMGAIYPGNGDTSYNQKFKGRVTMT |
| | | | RDTSTSTVYMELSSLRSEDTAVYYCAR |
| | | | SVYYSN.YWYFDLWGRGTLVTVSS |
| | | | (SEQ ID NO: 29) |
| | 2Lm15 | 2Hm | EIVLTQSPATLSLSPGERATLSCRASQSVSYIHW |
| | | | YQQKPGQAPRLLIYAPSNLASGIPARFSGSGSG |
| | | | TDFTLTISSLEPEDFAVYYCQQWISNPPTFGQG |
| | | | TKVEIKDGGGSGGGGSGGGGSSQVQLVQSGA |
| | | | EVKKPGASVKVSCKASGYTFTSYNMHWVRQ |
| | | | APGQGLEWMGAIYPGNGDTSYNQKFKGRVT |
| | | | MTRDTSTSTVYMELSSLRSEDTAVYYCAR |
| | | | SVYYSN.YWYFDLWGRGTLVTVSS |
| | | | (SEQ ID NO: 30) |
| | 2Lm16 | 2Hm3 | EIVLTQSPATLSLSPGERATLSCRASSSVSYMH |
| | | | WYQQKPGQAPRLLIYAPSNLASGIPARFSGSG |
| | | | SGTDFTLTISSLEPEDFAVYYCQQWSFNPPTFG |
| | | | QGTKVEIKDGGGSGGGGSGGGGSSQVQLVQS |
| | | | GAEVKKPGASVKVSCKASGYTFTSYNMHWV |
| | | | RQAPGQGLEWMGAIYPGNGDTSYNQKFKGR |
| | | | VTMTRDTSTSTVYMELSSLRSEDTAVYYCAR |
| | | | S.YYSNSYWYFDLWGRGTLVTVSS |
| | | | (SEQ ID NO: 31) |
| | 2Lm17-3 | 2Hm3 | EIVLTQSPATLSLSPGERATLSCRASQSVSYLS |
| | | | WYQQKPGQAPRLLIYAPSNLASGIPARFSGSG |
| | | | SGTDFTLTISSLEPEDFAVYYCQQWSFNPPTFG |
| | | | QGTKVEIKDGGGSGGGGSGGGGSSQVQLVQS |
| | | | GAEVKKPGASVKVSCKASGYTFTSYNMHWV |
| | | | RQAPGQGLEWMGAIYPGNGDTSYNQKFKGR |
| | | | VTMTRDTSTSTVYMELSSLRSEDTAVYYCA |
| | | | S,YYSNSYWYFDLWGRGTLVTVSS |
| | | | (SEQ ID NO: 32) |
| | 2Lm17-42 | Hm3 | EIVLTQSPATLSLSPGERATLSCRASQSVSYLT |
| | | | WYQQKPGQAPRLLIYAPSNLASGIPARFSGSG |
| | | | SGTDFTLTISSLEPEDFAVYYCQQWSFNPPTFG |
| | | | QGTKVEIKDGGGSGGGGSGGGGSSQVQLVQS |
| | | | GAEVKKPGASVKVSCKASGYTFTSYNMHWV |
| | | | RQAPGQGLEWMGAIYPGNGDTSYNQKFKGR |
| | | | VTMTRDTSTSTVYMELSSLRSEDTAVYYCAR |
| | | | S.YYSNSYWYFDLWGRGTLVTVSS |
| | | | (SEQ ID NO: 33) |
| | 2Lm17-6 | 2Hm3 | EIVLTQSPATLSLSPGERATLSCRASQSVSYLY |
| | | | WYQQKPGQAPRLLIYAPSNLASGIPARFSGSGS |
| | | | GTDFTLTISSLEPEDFAVYYCQQWSFNPPTFGQ |
| | | | GTKVEIKDGGGSGGGGSGGGGSSQVQLVQSGA |
| | | | EVKKPGASVKVSCKASGYTFTSYNMHWVRQA |
| | | | PGQGLEWMGAIYPGNGDTSYNQKFKGRVTMT |
| | | | RDTSTSTVYMELSSLRSEDTAVYYCAR |
| | | | S.YYSNSYWYFDLWGRGTLVTVSS |
| | | | (SEQ ID NO: 34) |
| | 2Lm17-8 | 2Hm3 | EIVLTQSPATLSLSPGERATLSCRASQSVSYLH |
| | | | WYQQKPGQAPRLLIYAPSNLASGIPARFSGSG |
| | | | SGTDFTLTISSLEPEDFAVYYCQQWSFNPPTFG |
| | | | QGTKVEIKDGGGSGGGGSGGGGSSQVQLVQS |
| | | | GAEVKKPGASVKVSCKASGYTFTSYNMHWV |
| | | | RQAPGQGLEWMGAIYPGNGDTSYNQKFKGR |
| | | | VTMTRDTSTSTVYMELSSLRSEDTAVYYCAR |
| | | | S.YYSNSYWYFDLWGRGTLVTVSS |
| | | | (SEQ ID NO: 35) |
| | 2Lm17- | 2Hm3 | EIVLTQSPATLSLSPGERATLSCRASQSVSYLN |
| | 12 | | WYQQKPGQAPRLLIYAPSNLASGIPARFSGSG |
| | | | SGTDFTLTISSLEPEDFAVYYCQQWSFNPPTF |
| | | | GQGTKVEIKDGGGSGGGGSGGGGSSQVQLV |
| | | | QSGAEVKKPGASVKVSCKASGYTFTSYNMH |
| | | | WVRQAPGQGLEWMGAIYPGNGDTSYNQKFK |
| | | | GRVTMTRDTSTSTVYMELSSLRSEDTAVYYCA |
| | | | RS.YYSNSYWYFDLWGRGTLVTVSS |
| | | | (SEQ ID NO: 36) |
| | 2Lm17- | 2Hm3 | EIVLTQSPATLSLSPGERATLSCRASQSVSYLA |
| | 14 | | WYQQKPGQAPRLLIYAPSNLASGIPARFSGSG |
| | | | SGTDFTLTISSLEPEDFAVYYCQQWSFNPPTFG |
| | | | QGTKVEIKDGGGSGGGGSGGGGSSQVQLVQS |
| | | | GAEVKKPGASVKVSCKASGYTFTSYNMHWVR |
| | | | QAPGQGLEWMGAIYPGNGDTSYNQKFKGRVT |
| | | | MTRDTSTSTVYMELSSLRSEDTAVYYCAR |
| | | | S.YYSNSYWYFDLWGRGTLVTVSS |
| | | | (SEQ ID NO: 37) |
| | 2Lm18-2 | 2Hm3 | EIVLTQSPATLSLSPGERATLSCRASSSVSYLA |
| | | | WYQQKPGQAPRLLIYAPSNLASGIPARFSGSG |
| | | | SGTDFTLTISSLEPEDFAVYYCQQWSFNPPTFG |
| | | | QGTKVEIKDGGGSGGGGSGGGGSSQVQLVQS |
| | | | GAEVKKPGASVKVSCKASGYTFTSYNMHWV |
| | | | RQAPGQGLEWMGAIYPGNGDTSYNQKFKGR |
| | | | VTMTRDTSTSTVYMELSSLRSEDTAVYYCAR |
| | | | S.YYSNSYWYFDLWGRGTLVTVSS |
| | | | (SEQ ID NO: 38) |
| | 2Lm18-3 | 2Hm3 | EIVLTQSPATLSLSPGERATLSCRASSSVSYLN |
| | | | WYQQKPGQAPRLLIYAPSNLASGIPARFSGS |
| | | | GSGTDFTLTISSLEPEDFAVYYCQQWSFNPPT |
| | | | FGQGTKVEIKDGGGSGGGGSGGGGSSQVQLV |
| | | | QSGAEVKKPGASVKVSCKASGYTFTSYNMH |
| | | | WVRQAPGQGLEWMGAIYPGNGDTSYNQKFKG |
| | | | RVTMTRDTSTSTVYMELSSLRSEDTAVYYCAR |
| | | | S.YYSNSYWYFDLWGRGTLVTVSS |
| | | | (SEQ ID NO: 39) |
| | 2Lm18-4 | 2Hm3 | EIVLTQSPATLSLSPGERATLSCRASSSVSYLD |
| | | | WYQQKPGQAPRLLIYAPSNLASGIPARFSGSG |
| | | | SGTDFTLTISSLEPEDFAVYYCQQWSFNPPTFG |
| | | | QGTKVEIKDGGGSGGGGSGGGGSSQVQLVQS |
| | | | GAEVKKPGASVKVSCKASGYTFTSYNMHWV |
| | | | RQAPGQGLEWMGAIYPGNGDTSYNQKFKGR |
| | | | VTMTRDTSTSTVYMELSSLRSEDTAVYYCAR |
| | | | S.YYSNSYWYFDLWGRGTLVTVSS |
| | | | (SEQ ID NO: 40) |
| | 2Lm18-5 | 2Hm3 | EIVLTQSPATLSLSPGERATLSCRASSSVSYLSW |
| | | | YQQKPGQAPRLLIYAPSNLASGIPARFSGSGSG |
| | | | TDFTLTISSLEPEDFAVYYCQQWSFNPPTFGQG |
| | | | TKVEIKDGGGSGGGGSGGGGSSQVQLVQSGAE |
| | | | VKKPGASVKVSCKASGYTFTSYNMHWVRQAP |
| | | | GQGLEWMGAIYPGNGDTSYNQKFKGRVTMTR |
| | | | DTSTSTVYMELSSLRSEDTAVYYCAR |
| | | | S.YYSNSYWYFDLWGRGTLVTVSS |
| | | | (SEQ ID NO: 41) |
| | 2Lm18- | 2Hm3 | EIVLTQSPATLSLSPGERATLSCRASSSVSYLHW |
| | 14 | | YQQKPGQAPRLLIYAPSNLASGIPARFSGSGSGT |
| | | | DFTLTISSLEPEDFAVYYCQQWSFNPPTFGQGTK |
| | | | VEIKDGGGSGGGGSGGGGSSQVQLVQSGAEVKK |
| | | | PGASVKVSCKASGYTFTSYNMHWVRQAPGQGL |
| | | | EWMGAIYPGNGDTSYNQKFKGRVTMTRDTSTST |
| | | | VYMELSSLRSEDTAVYYCAR |
| | | | S.YYSNSYWYFDLWGRGTLVTVSS |
| | | | (SEQ ID NO: 42) |
| | 2Lm19-1 | 2Hm3 | EIVLTQSPATLSLSPGERATLSCRASQSVSYIDW |
| | | | YQQKPGQAPRLLIYAPSNLASGIPARFSGSGSGT |
| | | | DFTLTISSLEPEDFAVYYCQQWSFNPPTFGQGTK |
| | | | VEIKDGGGSGGGGSGGGGSSQVQLVQSGAEVK |
| | | | KPGASVKVSCKASGYTFTSYNMHWVRQAPGQG |
| | | | LEWMGAIYPGNGDTSYNQKFKGRVTMTRDTST |
| | | | STVYMELSSLRSEDTAVYYCAR |
| | | | S.YYSNSYWYFDLWGRGTLVTVSS |
| | | | (SEQ ID NO: 43) |
| | 2Lm19-2 | 2Hm3 | EIVLTQSPATLSLSPGERATLSCRASQSVSYISW |
| | | | YQQKPGQAPRLLIYAPSNLASGIPARFSGSGSG |
| | | | TDFTLTISSLEPEDFAVYYCQQWSFNPPTFGQG |
| | | | TKVEIKDGGGSGGGGSGGGGSSQVQLVQSGAE |
| | | | VKKPGASVKVSCKASGYTFTSYNMHWVRQAP |
| | | | GQGLEWMGAIYPGNGDTSYNQKFKGRVTMTR |
| | | | DTSTSTVYMELSSLRSEDTAVYYCAR |
| | | | S.YYSNSYWYFDLWGRGTLVTVSS |
| | | | (SEQ ID NO: 44) |
| | 2Lm19-3 | 2Hm3 | EIVLTQSPATLSLSPGERATLSCRASQSVSYIVW |
| | | | YQQKPGQAPRLLIYAPSNLASGIPARFSGSGSGT |
| | | | DFTLTISSLEPEDFAVYYCQQWSFNPPTFGQGT |
| | | | KVEIKDGGGSGGGGSGGGGSSQVQLVQSGAEV |
| | | | KKPGASVKVSCKASGYTFTSYNMHWVRQAPG |
| | | | QGLEWMGAIYPGNGDTSYNQKFKGRVTMTRD |
| | | | TSTSTVYMELSSLRSEDTAVYYCAR |
| | | | S.YYSNSYWYFDLWGRGTLVTVSS |
| | | | (SEQ ID NO: 45) |
| | 2Lm19-4 | 2Hm3 | EIVLTQSPATLSLSPGERATLSCRASQSVSYIAW |
| | | | YQQKPGQAPRLLIYAPSNLASGIPARFSGSGSG |
| | | | TDFTLTISSLEPEDFAVYYCQQWSFNPPTFGQG |
| | | | TKVEIKDGGGSGGGGSGGGGSSQVQLVQSGAE |
| | | | VKKPGASVKVSCKASGYTFTSYNMHWVRQAP |
| | | | GQGLEWMGAIYPGNGDTSYNQKFKGRVTMTR |
| | | | DTSTSTVYMELSSLRSEDTAVYYCAR |
| | | | S.YYSNSYWYFDLWGRGTLVTVSS |
| | | | (SEQ ID NO: 46) |
| | 2Lm19-7 | 2Hm3 | EIVLTQSPATLSLSPGERATLSCRASQSVSYITW |
| | | | YQQKPGQAPRLLIYAPSNLASGIPARFSGSGSG |
| | | | TDFTLTISSLEPEDFAVYYCQQWSFNPPTFGQG |
| | | | TKVEIKDGGGSGGGGSGGGGSSQVQLVQSGAE |
| | | | VKKPGASVKVSCKASGYTFTSYNMHWVRQAP |
| | | | GQGLEWMGAIYPGNGDTSYNQKFKGRVTMTR |
| | | | DTSTSTVYMELSSLRSEDTAVYYCAR |
| | | | S.YYSNSYWYFDLWGRGTLVTVSS |
| | | | (SEQ ID NO: 47) |
| | 2Lm19-9 | 2Hm3 | EIVLTQSPATLSLSPGERATLSCRASQSVSYIIW |
| | | | YQQKPGQAPRLLIYAPSNLASGIPARFSGSGSG |
| | | | TDFTLTISSLEPEDFAVYYCQQWSFNPPTFGQG |
| | | | TKVEIKDGGGSGGGGSGGGGSSQVQLVQSGAE |
| | | | VKKPGASVKVSCKASGYTFTSYNMHWVRQAP |
| | | | GQGLEWMGAIYPGNGDTSYNQKFKGRVTMTR |
| | | | DTSTSTVYMELSSLRSEDTAVYYCAR |
| | | | S.YYSNSYWYFDLWGRGTLVTVSS |
| | | | (SEQ ID NO: 48) |
| | 2Lm19- | 2Hm3 | EIVLTQSPATLSLSPGERATLSCRASQSVSYIPW |
| | 12 | | YQQKPGQAPRLLIYAPSNLASGIPARFSGSGSG |
| | | | TDFTLTISSLEPEDFAVYYCQQWSFNPPTFGQG |
| | | | TKVEIKDGGGSGGGGSGGGGSSQVQLVQSGAE |
| | | | VKKPGASVKVSCKASGYTFTSYNMHWVRQAP |
| | | | GQGLEWMGAIYPGNGDTSYNQKFKGRVTMTR |
| | | | DTSTSTVYMELSSLRSEDTAVYYCAR |
| | | | S.YYSNSYWYFDLWGRGTLVTVSS |
| | | | (SEQ ID NO: 49) |
| | 2Lm19- | 2Hm3 | EIVLTQSPATLSLSPGERATLSCRASQSVSYINW |
| | 14 | | YQQKPGQAPRLLIYAPSNLASGIPARFSGSGSG |
| | | | TDFTLTISSLEPEDFAVYYCQQWSFNPPTFGQG |
| | | | TKVEIKDGGGSGGGGSGGGGSSQVQLVQSGAE |
| | | | VKKPGASVKVSCKASGYTFTSYNMHWVRQAP |
| | | | GQGLEWMGAIYPGNGDTSYNQKFKGRVTMTR |
| | | | DTSTSTVYMELSSLRSEDTAVYYCAR |
| | | | S.YYSNSYWYFDLWGRGTLVTVSS |
| | | | (SEQ ID NO: 50) |
| | 2Lm20-1 | 2Hm3 | EIVLTQSPATLSLSPGERATLSCRASSSVSYISW |
| | | | YQQKPGQAPRLLIYAPSNLASGIPARFSGSGSG |
| | | | TDFTLTISSLEPEDFAVYYCQQWSFNPPTFGQG |
| | | | TKVEIKDGGGSGGGGSGGGGSSQVQLVQSGAE |
| | | | VKKPGASVKVSCKASGYTFTSYNMHWVRQAP |
| | | | GQGLEWMGAIYPGNGDTSYNQKFKGRVTMTR |
| | | | DTSTSTVYMELSSLRSEDTAVYYCAR |
| | | | S.YYSNSYWYFDLWGRGTLVTVSS |
| | | | (SEQ ID NO: 51) |
| | 2Lm20-2 | 2Hm3 | EIVLTQSPATLSLSPGERATLSCRASSSVSYIAW |
| | | | YQQKPGQAPRLLIYAPSNLASGIPARFSGSGSG |
| | | | TDFTLTISSLEPEDFAVYYCQQWSFNPPTFGQG |
| | | | TKVEIKDGGGSGGGGSGGGGSSQVQLVQSGAE |
| | | | VKKPGASVKVSCKASGYTFTSYNMHWVRQAP |
| | | | GQGLEWMGAIYPGNGDTSYNQKFKGRVTMTR |
| | | | DTSTSTVYMELSSLRSEDTAVYYCAR |
| | | | S.YYSNSYWYFDLWGRGTLVTVSS |
| | | | (SEQ ID NO: 52) |
| | 2Lm20-4 | 2Hm3 | EIVLTQSPATLSLSPGERATLSCRASSSVSYIVW |
| | | | YQQKPGQAPRLLIYAPSNLASGIPARFSGSGSG |
| | | | TDFTLTISSLEPEDFAVYYCQQWSFNPPTFGQG |
| | | | TKVEIKDGGGSGGGGSGGGGSSQVQLVQSGAE |
| | | | VKKPGASVKVSCKASGYTFTSYNMHWVRQAP |
| | | | GQGLEWMGAIYPGNGDTSYNQKFKGRVTMTR |
| | | | DTSTSTVYMELSSLRSEDTAVYYCAR |
| | | | S.YYSNSYWYFDLWGRGTLVTVSS |
| | | | (SEQ ID NO: 53) |
| | 2Lm20-8 | 2Hm3 | EIVLTQSPATLSLSPGERATLSCRASSSVNYIYW |
| | | | YQQKPGQAPRLLIYAPSNLASGIPARFSGSGSG |
| | | | TDFTLTISSLEPEDFAVYYCQQWSFNPPTFGQG |
| | | | TKVEIKDGGGSGGGGSGGGGSSQVQLVQSGAE |
| | | | VKKPGASVKVSCKASGYTFTSYNMHWVRQAP |
| | | | GQGLEWMGAIYPGNGDTSYNQKFKGRVTMTR |
| | | | DTSTSTVYMELSSLRSEDTAVYYCAR |
| | | | S.YYSNSYWYFDLWGRGTLVTVSS |
| | | | (SEQ ID NO: 54) |
| | 2Lm20- | 2Hm3 | EIVLTQSPATLSLSPGERATLSCRASSSVSYIDW |
| | 11 | | YQQKPGQAPRLLIYAPSNLASGIPARFSGSGSG |
| | | | TDFTLTISSLEPEDFAVYYCQQWSFNPPTFGQG |
| | | | TKVEIKDGGGSGGGGSGGGGSSQVQLVQSGAE |
| | | | VKKPGASVKVSCKASGYTFTSYNMHWVRQAP |
| | | | GQGLEWMGAIYPGNGDTSYNQKFKGRVTMTR |
| | | | DTSTSTVYMELSSLRSEDTAVYYCAR |
| | | | S.YYSNSYWYFDLWGRGTLVTVSS |
| | | | (SEQ ID NO: 55) |
| | 2Lm20- | 2Hm3 | EIVLTQSPATLSLSPGERATLSCRASSSVSYIIW |
| | 12 | | YQQKPGQAPRLLIYAPSNLASGIPARFSGSGSG |
| | | | TDFTLTISSLEPEDFAVYYCQQWSFNPPTFGQG |
| | | | TKVEIKDGGGSGGGGSGGGGSSQVQLVQSGAE |
| | | | VKKPGASVKVSCKASGYTFTSYNMHWVRQAP |
| | | | GQGLEWMGAIYPGNGDTSYNQKFKGRVTMTR |
| | | | DTSTSTVYMELSSLRSEDTAVYYCAR |
| | | | S.YYSNSYWYFDLWGRGTLVTVSS |
| | | | (SEQ ID NO: 56) |
| | 2Lm20- | 2Hm3 | EIVLTQSPATLSLSPGERATLSCRASSSVSYIYW |
| | 13 | | YQQKPGQAPRLLIYAPSNLASGIPARFSGSGSG |
| | | | TDFTLTISSLEPEDFAVYYCQQWSFNPPTFGQG |
| | | | TKVEIKDGGGSGGGGSGGGGSSQVQLVQSGAE |
| | | | VKKPGASVKVSCKASGYTFTSYNMHWVRQAP |
| | | | GQGLEWMGAIYPGNGDTSYNQKFKGRVTMTR |
| | | | DTSTSTVYMELSSLRSEDTAVYYCAR |
| | | | S.YYSNSYWYFDLWGRGTLVTVSS |
| | | | (SEQ ID NO: 57) |
| | 2Lm5 | 2H5m3 | EIVLTQSPATLSLSPGERATLSCRASQSVSYMH |
| | (18009) | | WYQQKPGQAPRLLIYAPSNLASGIPARFSGS |
| | | | GSGTDFTLTISSLEPEDFAVYYCQQWSFNPPT |
| | | | FGQGTKVEIKDGGGSGGGGSGGGGTGEVQLV |
| | | | QSGAEVKKPGESLKISCKGSGYSFTSYNMHW |
| | | | VRQMPGKGLEWMGAIYPGNGDTSYNQKFKG |
| | | | QVTISADKSISTAYLQWSSLKASDTAMYYCA |
| | | | RVVYYSNSYWYFDLWGRGTLVTVSS |
| | | | (SEQ ID NO: 58) |
| | 2Lm5 | 2H3m3 | EIVLTQSPATLSLSPGERATLSCRASQSVSYMH |
| | (2Lm5 | | WYQQKPGQAPRLLIYAPSNLASGIPARFSGSGS |
| | 2H3m3) | | GTDFTLTISSLEPEDFAVYYCQQWSFNPPTFG |
| | | | QGTKVEIKDGGGSGGGGSGGGGTGEVQLLES |
| | | | GGGLVQPGGSLRLSCAASGFTFSSYNMHWVR |
| | | | QAPGKGLEWVSAIYPGNGDTSYNQKFKGRFT |
| | | | ISRDNSKNTLYLQMNSLRAEDTAVYYCA |
| | | | KSYYSNSYWYFDLWGRGTLVTVSS |
| | | | (SEQ ID NO: 59) |
| | IgG1 | Hinge | DQEPKSCDKTHTSPPSS |
| | | CSSS | (SEQ ID NO: 60) |
| | IgG1 | Hinge | DQEPKSCDKTHTCPPCP |
| | | WT | (SEQ ID NO: 61) |
| | IgG1 | Hinge | DQEPKSCDKTHTSPPCS |
| | | CSCS | (SEQ ID NO: 62) |
| | IgG1 | Hinge | DQEPKSSDKTHTCPPCS |
| | | SCCS | (SEQ ID NO: 63) |
| | IgG1 | Hinge | DQEPKSSDKTHTCPPCP |
| | | SCCP | (SEQ ID NO: 64) |
| | IgG1 | CH2CH | APELLGGPSVFLFPPKPKDTLMISRTPEVTCVVVDVSHEDPEVKF |
| | | 3 | N |
| | | WT | WYVDGVEVHNAKTKPREEQYNSTYRVVSVLTVLHQDWLNGKE |
| | | | YKCKVSNKALPAPIEKTISKAKGQPREPQVYTLPPSRDELTKNQV |
| | | | SLTCLVKGFYPSDIAVEWESNGQPENNYKTTPPVLDSDGSFFLYS |
| | | | KLTVDKSRWQQGNVFSCSVMHEALHNHYTQKSLSLSPGK |
| | | | (SEQ ID NO: 65) |
| | IgG1 | CH2CH | APELLGGPSVFLFPPKPKDTLMISRTPEVTCVVVDVSHEDPEVKF |
| | | 3 | N |
| | | P331S | WYVDGVEVHNAKTKPREEQYNSTYRVVSVLTVLHQDWLNGKE |
| | | | YKCKVSNKALPASIEKTISKAKGQPREPQVYTLPPSRDELTKNQV |
| | | | SLTCLVKGFYPSDIAVEWESNGQPENNYKTTPPVLDSDGSFFLYS |
| | | | KLTVDKSRWQQGNVFSCSVMHEALHNHYTQKSLSLSPGK |
| | | | (SEQ ID NO: 66) |

Exemplary Full Length

| | |
|---|---|
| SEQ ID NO: 67 | EIVLTQSPATLSLSPGERATLSCRASQSVSYIVWYQQKPGQAPRL |
| | LIYAPSNLASGIPARFSGSGSGTDFTLTISSLEPEDFAVYYCQQWS |
| | FNPPTFGQGTKVEIKDGGGSGGGGSGGGGTGEVQLVQSGAEVK |
| | KPGESLKISCKGSGYSFTSYNMHWVRQMPGKGLEWMGAIYPGN |
| | GDTSYNQKFKGQVTISADKSISTAYLQWSSLKASDTAMYYCARS |
| | YYSNSYWYFDLWGRGTLVTVSSDQEPKSSDKTHTCPPCPAPELL |
| | GGPSVFLFPPKPKDTLMISRTPEVTCVVVDVSHEDPEVKFNWYV |
| | DGVEVHNAKTKPREEQYNSTYRVVSVLTVLHQDWLNGKEYKC |
| | KVSNKALPASIEKTISKAKGQPREPQVYTLPPSRDELTKNQVSLT |
| | CLVKGFYPSDIAVEWESNGQPENNYKTTPPVLDSDGSFFLYSKLT |
| | VDKSRWQQGNVFSCSVMHEALHNHYTQKSLSLSPGK |
| SEQ ID NO: 68 | EIVLTQSPATLSLSPGERATLSCRASSSVSYIVWYQQKPGQAPRLL |
| | IYAPSNLASGIPARFSGSGSGTDFTLTISSLEPEDFAVYYCQQWSF |
| | NPPTFGQGTKVEIKDGGGSGGGGSGGGGSSQVQLVQSGAEVKK |
| | PGASVKVSCKASGYTFTSYNMHWVRQAPGQGLEWMGAIYPGN |
| | GDTSYNQKFKGRVTMTRDTSTSTVYMELSSLRSEDTAVYYCARS |
| | .YYSNSYWYFDLWGRGTLVTVSSDQEPKSSDKTHTCPPCPAPELL |
| | GGPSVFLFPPKPKDTLMISRTPEVTCVVVDVSHEDPEVKFNWYV |
| | DGVEVHNAKTKPREEQYNSTYRVVSVLTVLHQDWLNGKEYKC |
| | KVSNKALPASIEKTISKAKGQPREPQVYTLPPSRDELTKNQVSLT |
| | CLVKGFYPSDIAVEWESNGQPENNYKTTPPVLDSDGSFFLYSKLT |
| | VDKSRWQQGNVFSCSVMHEALHNHYTQKSLSLSPGK |
| SEQ ID NO: 69 | QIVLSQSPAILSASPGEKVTMTCRASSSVSYMHWYQQKPGSSPKP |
| | WIYAPSNLASGVPARFSGSGSGTSYSLTISRVEAEDAATYYCQQ |
| | WSFNPPTFGAGTKLELKDGGGSGGGGSGGGGSSQAYLQQSGAE |
| | SVRPGASVKMSCKASGYTFTSYNMHWVKQTPRQGLEWIGAIYP |
| | GNGDTSYNQKFKGKATLTVDKSSSTAYMQLSSLTSEDSAVYFCA |
| | RVVYYSNSYWYFDVWGTGTTVTVSDQEPKSCDKTHTSPPCSAP |
| | ELLGGPSVFLFPPKPKDTLMISRTPEVTCVVVDVSHEDPEVKFNW |
| | YVDGVEVHNAKTKPREEQYNSTYRVVSVLTVLHQDWLNGKEY |
| | KCKVSNKALPAPIEKTISKAKGQPREPQVYTLPPSRDELTKNQVS |
| | LTCLVKGFYPSDIAVEWESNGQPENNYKTTPPVLDSDGSFFLYSK |
| | LTVDKSRWQQGNVFSCSVMHEALHNHYTQKSLSLSPGK |
| SEQ ID NO: 70 | EIVLTQSPATLSLSPGERATLSCRASQSVSYIVWYQQKPGQAPRL |
| | LIYAPSNLASGIPARFSGSGSGTDFTLTISSLEPEDFAVYYCQQWS |
| | FNPPTFGQGTKVEIKDGGGSGGGGSGGGGTGEVQLVQSGAEVK |
| | KPGESLKISCKGSGYSFTSYNMHWVRQMPGKGLEWMGAIYPGN |
| | GDTSYNQKFKGQVTISADKSISTAYLQWSSLKASDTAMYYCARS |
| | YYSNSYWYFDLWGRGTLVTVSSDQEPKSSDKTHTCPPCPAPELL |
| | GGPSVFLFPPKPKDTLMISRTPEVTCVVVDVSHEDPEVKFNWYV |
| | DGVEVHNAKTKPREEQYNSTYRVVSVLTVLHQDWLNGKEYKC |
| | KVSNKALPAPIEKTISKAKGQPREPQVYTLPPSRDELTKNQVSLT |
| | CLVKGFYPSDIAVEWESNGQPENNYKTTPPVLDSDGSFFLYSKLT |
| | VDKSRWQQGNVFSCSVMHEALHNHYTQKSLSLSPGK |
| SEQ ID NO: 71 | EIVLTQSPATLSLSPGERATLSCRASSSVSYIVWYQQKPGQAPRLL |
| | IYAPSNLASGIPARFSGSGSGTDFTLTISSLEPEDFAVYYCQQWSF |
| | NPPTFGQGTKVEIKDGGGSGGGGSGGGGSSQVQLVQSGAEVKK |
| | PGASVKVSCKASGYTFTSYNMHWVRQAPGQGLEWMGAIYPGN |
| | GDTSYNQKFKGRVTMTRDTSTSTVYMELSSLRSEDTAVYYCARS |
| | .YYSNSYWYFDLWGRGTLVTVSSDQEPKSSDKTHTCPPCPAPELL |
| | GGPSVFLFPPKPKDTLMISRTPEVTCVVVDVSHEDPEVKFNWYV |
| | DGVEVHNAKTKPREEQYNSTYRVVSVLTVLHQDWLNGKEYKC |
| | KVSNKALPAPIEKTISKAKGQPREPQVYTLPPSRDELTKNQVSLT |
| | CLVKGFYPSDIAVEWESNGQPENNYKTTPPVLDSDGSFFLYSKLT |
| | VDKSRWQQGNVFSCSVMHEALHNHYTQKSLSLSPGK |
| SEQ ID NO: 72 | EIVLTQSPATLSLSPGERATLSCRASSSVSYIDWYQQKPGQAPRLL |
| | IYAPSNLASGIPARFSGSGSGTDFTLTISSLEPEDFAVYYCQQWSF |
| | NPPTFGQGTKVEIKDGGGSGGGGSGGGGSSQVQLVQSGAEVKK |
| | PGASVKVSCKASGYTFTSYNMHWVRQAPGQGLEWMGAIYPGN |
| | GDTSYNQKFKGRVTMTRDTSTSTVYMELSSLRSEDTAVYYCARS |
| | YYSNSYWYFDLWGRGTLVTVSSDQEPKSCDKTHTSPPSSAPELL |
| | GGPSVFLFPPKPKDTLMISRTPEVTCVVVDVSHEDPEVKFNWYV |
| | DGVEVHNAKTKPREEQYNSTYRVVSVLTVLHQDWLNGKEYKC |
| | KVSNKALPAPIEKTISKAKGQPREPQVYTLPPSRDELTKNQVSLT |
| | CLVKGFYPSDIAVEWESNGQPENNYKTTPPVLDSDGSFFLYSKLT |
| | VDKSRWQQGNVFSCSVMHEALHNHYTQKSLSLSPGK |
| SEQ ID NO: 73 | EIVLTQSPATLSLSPGERATLSCRASSSVSYIVWYQQKPGQAPRLL |
| | IYAPSNLASGIPARFSGSGSGTDFTLTISSLEPEDFAVYYCQQWSF |
| | NPPTFGQGTKVEIKDGGGSGGGGSGGGGSSQVQLVQSGAEVKK |
| | PGASVKVSCKASGYTFTSYNMHWVRQAPGQGLEWMGAIYPGN |
| | GDTSYNQKFKGRVTMTRDTSTSTVYMELSSLRSEDTAVYYCARS |
| | YYSNSYWYFDLWGRGTLVTVSSDQEPKSSDKTHTCPPCPAPELL |
| | GGPSVFLFPPKPKDTLMISRTPEVTCVVVDVSHEDPEVKFNWYV |
| | DGVEVHNAKTKPREEQYNSTYRVVSVLTVLHQDWLNGKEYKC |
| | KVSNKALPAPIEKTISKAKGQPREPQVYTLPPSRDELTKNQVSLT |
| | CLVKGFYPSDIAVEWESNGQPENNYKTTPPVLDSDGSFFLYSKLT |
| | VDKSRWQQGNVFSCSVMHEALHNHYTQKSLSLSPGK |
| SEQ ID NO: 74 | EIVLTQSPATLSLSPGERATLSCRASQSVSYIVWYQQKPGQAPRL |
| | LIYAPSNLASGIPARFSGSGSGTDFTLTISSLEPEDFAVYYCQQWS |
| | FNPPTFGQGTKVEIKDGGGSGGGGSGGGGTGEVQLVQSGAEVK |
| | KPGESLKISCKGSGYSFTSYNMHWVRQMPGKGLEWMGAIYPGN |
| | GDTSYNQKFKGQVTISADKSISTAYLQWSSLKASDTAMYYCARV |
| | VYYSNSYWYFDLWGRGTLVTVSSDQEPKSCDKTHTSPPCSAPEL |
| | LGGPSVFLFPPKPKDTLMISRTPEVTCVVVDVSHEDPEVKFNWY |
| | VDGVEVHNAKTKPREEQYNSTYRVVSVLTVLHQDWLNGKEYK |
| | CKVSNKALPASIEKTISKAKGQPREPQVYTLPPSRDELTKNQVSL |
| | TCLVKGFYPSDIAVEWESNGQPENNYKTTPPVLDSDGSFFLYSKL |
| | TVDKSRWQQGNVFSCSVMHEALHNHYTQKSLSLSPGK |
| SEQ ID NO: 75 | EIVLTQSPATLSLSPGERATLSCRASSSVSYMIWYQQKPGQAPRL |
| | LIYAISNLASGIPARFSGSGSGTDFTLTISSLEPEDFAVYYCQQWIS |
| | NPLTFGQGTKVEIKDGGGSGGGGSGGGGSSQVQLVQSGAEVKK |
| | PGASVKVSCKASGYTFTSYNMHWVRQAPGQGLEWMGAIYPGN |
| | GDTSYNQKFKGRVTMTRDTSTSTVYMELSSLRSEDTAVYYCARS |
| | VYYSN.YWYFDLWGRGTLVTVSSDQEPKSCDKTHTCPPCPAPEL |
| | LGGPSVFLFPPKPKDTLMISRTPEVTCVVVDVSHEDPEVKFNWY |
| | VDGVEVHNAKTKPREEQYNSTYRVVSVLTVLHQDWLNGKEYK |
| | CKVSNKALPAPIEKTISKAKGQPREPQVYTLPPSRDELTKNQVSL |
| | TCLVKGFYPSDIAVEWESNGQPENNYKTTPPVLDSDGSFFLYSKL |
| | TVDKSRWQQGNVFSCSVMHEALHNHYTQKSLSLSPGK |
| SEQ ID NO: 76 | EIVLTQSPATLSLSPGERATLSCRASSSVSYIIWYQQKPGQAPRLLI |
| | YAPSNLASGIPARFSGSGSGTDFTLTISSLEPEDFAVYYCQQWSFN |
| | PPTFGQGTKVEIKDGGGSGGGGSGGGGSSQVQLVQSGAEVKKP |
| | GASVKVSCKASGYTFTSYNMHWVRQAPGQGLEWMGAIYPGNG |
| | DTSYNQKFKGRVTMTRDTSTSTVYMELSSLRSEDTAVYYCARSY |
| | YSNSYWYFDLWGRGTLVTVSSDQEPKSCDKTHTSPPSSAPELLG |
| | GPSVFLFPPKPKDTLMISRTPEVTCVVVDVSHEDPEVKFNWYVD |
| | GVEVHNAKTKPREEQYNSTYRVVSVLTVLHQDWLNGKEYKCK |
| | VSNKALPASIEKTISKAKGQPREPQVYTLPPSRDELTKNQVSLTCL |
| | VKGFYPSDIAVEWESNGQPENNYKTTPPVLDSDGSFFLYSKLTV |
| | DKSRWQQGNVFSCSVMHEALHNHYTQKSLSLSPGK |

### EQUIVALENTS

The foregoing has been a description of certain non-limiting embodiments of the invention. Those skilled in the art will recognize, or be able to ascertain using no more than routine experimentation, many equivalents to the specific embodiments of the invention described herein. Those of ordinary skill in the art will appreciate that various changes and modifications to this description may be made without departing from the spirit or scope of the present invention, as defined in the following claims.

In the claims articles such as “a,”, “an” and “the” may mean one or more than one unless indicated to the contrary or otherwise evident from the context. Claims or descriptions that include “or” between one or more members of a group are considered satisfied if one, more than one, or all of the group members are present in, employed in, or otherwise relevant to a given product or process unless indicated to the contrary or otherwise evident from the context. The invention includes embodiments in which exactly one member of the group is present in, employed in, or otherwise relevant to a given product or process. The invention also includes embodiments in which more than one, or all of the group members are present in, employed in, or otherwise relevant to a given product or process. Furthermore, it is to be understood that the invention encompasses all variations, combinations, and permutations in which one or more limitations, elements, clauses, descriptive terms, etc., from one or more of the claims or from relevant portions of the description is introduced into another claim. For example, any claim that is dependent on another claim can be modified to include one or more limitations found in any other claim that is dependent on the same base claim. Furthermore, where the claims recite a composition, it is to be understood that methods of using the composition for any of the purposes disclosed herein are included, and methods of making the composition according to any of the methods of making disclosed herein or other methods known in the art are included, unless otherwise indicated or unless it would be evident to one of ordinary skill in the art that a contradiction or inconsistency would arise. In addition, the invention encompasses compositions made according to any of the methods for preparing compositions disclosed herein.

Where elements are presented as lists, e.g., in Markush group format, it is to be understood that each subgroup of the elements is also disclosed, and any element(s) can be removed from the group. It is also noted that the term “comprising” is intended to be open and permits the inclusion of additional elements or steps. It should be understood that, in general, where the invention, or aspects of the invention, is/are referred to as comprising particular elements, features, steps, etc., certain embodiments of the invention or aspects of the invention consist, or consist essentially of, such elements, features, steps, etc. For purposes of simplicity those embodiments have not been specifically set forth in haec verba herein. Thus for each embodiment of the invention that comprises one or more elements, features, steps, etc., the invention also provides embodiments that consist or consist essentially of those elements, features, steps, etc.

Where ranges are given, endpoints are included. Furthermore, it is to be understood that unless otherwise indicated or otherwise evident from the context and/or the understanding of one of ordinary skill in the art, values that are expressed as ranges can assume any specific value within the stated ranges in different embodiments of the invention, to the tenth of the unit of the lower limit of the range, unless the context clearly dictates otherwise. It is also to be understood that unless otherwise indicated or otherwise evident from the context and/or the understanding of one of ordinary skill in the art, values expressed as ranges can assume any subrange within the given range, wherein the endpoints of the subrange are expressed to the same degree of accuracy as the tenth of the unit of the lower limit of the range.

In addition, it is to be understood that any particular embodiment of the present invention may be explicitly excluded from any one or more of the claims. Any embodiment, element, feature, application, or aspect of the compositions and/or methods of the invention can be excluded from any one or more claims. For purposes of brevity, all of the embodiments in which one or more elements, features, purposes, or aspects is excluded are not set forth explicitly herein.

### Incorporation by Reference

All publications and patent documents cited in this application are incorporated by reference in their entirety for all purposes to the same extent as if the contents of each individual publication or patent document were incorporated herein.

What is claimed is:

1-62. (canceled)

63. A formulation comprising a lyophilized mixture of a small modular immunopharmaceutical protein wherein less than 7% of the lyophilized small modular immunopharmaceutical protein exists in aggregated form.

64. The formulation of claim 63, further comprising a bulking agent selected from the group consisting of sucrose, mannitol, glycine, sodium chloride, dextran, trehalose and combinations thereof and/or a buffering agent selected from the group consisting of histidine, sodium acetate, citrate, phosphate, succinate, Tris and combinations thereof.

65. The formulation of claim 64, wherein the formulation further comprises a stabilizing agent selected from the group consisting of sucrose, sorbitol, mannitol, glycine, trehalose and combinations thereof.

66. The formulation of claim 65, wherein the formulation further comprises an isotonicity agent selected from the group consisting of glycine, sorbitol, sucrose, mannitol, sodium chloride, dextrose, arginine and combinations thereof.

67. The formulation of claim 65, wherein the formulation further comprises a surfactant selected from the group consisting of Polysorbate 20, Polysorbate 80, poloxamers, Triton and combinations thereof.

68. The formulation of claim 63 comprising a lyophilized mixture of a small modular immunopharmaceutical protein, sucrose, histidine, methionine, and Polysorbate 80.

69. The formulation of claim 68, comprising about 50 mg/ml small modular immunopharmaceutical protein, about 10 mM histidine, about 10 mM methionine, about 5% sucrose, and about 0.01% Polysorbate 80.

70. The formulation of claim 68, comprising about 100 mg/ml small modular immunopharmaceutical protein, about 20 mM histidine, about 10 mM methionine, about 10% sucrose, and about 0.01% Polysorbate 80.

71. The formulation of claim 68, wherein the small modular immunopharmaceutical protein comprises a binding domain that specifically targets CD20 comprising an amino acid sequence having at least 80% identity to any one of SEQ ID NOs: 1-59 and 67-76.

72. The formulation of claim 68, wherein the lyophilized small modular immunopharmaceutical protein is stable at room temperature.

73. A kit comprising a container which holds the formulation of claim 68.

74. A reconstituted formulation comprising the formulation of claim 68 reconstituted with a diluent, wherein the small modular immunopharmaceutical protein is present in the reconstituted formulation at a concentration within a range from 25 mg/ml to 400 mg/ml.

75. The reconstituted formulation of claim 74 for intravenous, subcutaneous, or intramuscular administration.

76. Use of the reconstituted formulation of claim 74 for treating a patient.

77. A formulation for lyophilization comprising a small modular immunopharmaceutical protein, a non-reducing sugar, and a buffering agent.

78. The formulation of claim 77, wherein the buffering agent is at a concentration of approximately 10 to 20 mM.

79. The formulation of claim 77, further comprising methionine.

80. The formulation of claim 77, wherein the non-reducing sugar is sucrose at a concentration between 1% and 10%.

81. The formulation of claim 80, wherein the formulation further comprises a surfactant selected from the group consisting of Polysorbate 20, Polysorbate 80, poloxamers, Triton and combinations thereof.

82. The formulation of claim 81, wherein the sucrose is at a concentration ranging between approximately 5% and 10%, further comprising histidine at a concentration ranging between approximately 10 mM and 20 mM, further comprising methionine is a concentration ranging between approximately 10 mM and 20 mM, and wherein Polysorbate 80 is at a concentration ranging between approximately 0.001% and 0.1%.

83. The formulation of claim 82, wherein the small modular immunopharmaceutical protein is at a concentration ranging between approximately 25 mg/ml and 400 mg/ml.

84. The formulation of claim 83, wherein the small modular immunopharmaceutical protein is at a concentration of approximately 100 mg/ml, sucrose is at a concentration of approximately 10%, histidine is at a concentration of approximately 20 mM, and Polysorbate-80 is at a concentration of approximately 0.01%.

85. The formulation of claim 83, wherein the small modular immunopharmaceutical protein is at a concentration of approximately 50 mg/ml, sucrose is at a concentration of approximately 5%, methionine is at a concentration of approximately 10 mM, histidine is at a concentration of approximately 20 mM, and Polysorbate-80 is at a concentration of approximately 0.01%.

86. The formulation of claim 84, wherein the formulation has a pH of 6.0.

87. The formulation of claim 77, wherein the small modular immunopharmaceutical protein comprises a binding domain that specifically targets CD20 and comprises an amino acid sequence having at least 80% identity to any one of SEQ ID NOs: 1-59 and 67-76.

88. A method of storing a small modular immunopharmaceutical protein comprising: lyophilizing a formulation comprising a small modular immunopharmaceutical protein according to claim 77; and storing the lyophilized formulation at a temperature at or lower than room temperature.


SEQUENCE LISTING (76 sequences)

SEQ ID NO: 1 - protein, 243 aa, Artificial Sequence
  SMP18011
EIVLTQSPATLSLSPGERATLSCRASQSVSYIVWYQQKPGQAPRLLIYAPSNLASGIPARFSGSGSGTDFTLTISSLEPEDFAVYYCQQWSFNPPTFGQGTKVEIKDGGGSGGGGSGGGGTGEVQLVQSGAEVKKPGESLKISCKGSGYSFTSYNMHWVRQMPGKGLEWMGAIYPGNGDTSYNQKFKGQVTISADKSISTAYLQWSSLKASDTAMYYCARSYYSNSYWYFDLWGRGTLVTVSS

SEQ ID NO: 2 - protein, 244 aa, Artificial Sequence
  SMP18008
EIVLTQSPATLSLSPGERATLSCRASQSVSYMHWYQQKPGQAPRLLIYAPSNLASGIPARFSGSGSGTDFTLTISSLEPEDFAVYYCQQWSFNPPTFGQGTKVEIKDGGGSGGGGSGGGGTGEVQLVQSGAEVKKPGESLKISCKGSGYSFTSYNMHWVRQMPGKGLEWMGAIYPGNGDTSYNQKFKGQVTISADKSISTAYLQWSSLKASDTAMYYCARVVYYSNSYWYFDLWGRGTLVTVSS

SEQ ID NO: 3 - protein, 244 aa, Artificial Sequence
  SMP18010
EIVLTQSPATLSLSPGERATLSCRASQSVSYIVWYQQKPGQAPRLLIYAPSNLASGIPARFSGSGSGTDFTLTISSLEPEDFAVYYCQQWSFNPPTFGQGTKVEIKDGGGSGGGGSGGGGTGEVQLVQSGAEVKKPGESLKISCKGSGYSFTSYNMHWVRQMPGKGLEWMGAIYPGNGDTSYNQKFKGQVTISADKSISTAYLQWSSLKASDTAMYYCARVVYYSNSYWYFDLWGRGTLVTVSS

SEQ ID NO: 4 - protein, 244 aa, Artificial Sequence; identical to SEQ ID NO: 3
  SMP18009

SEQ ID NO: 5 - protein, 244 aa, Artificial Sequence
  SMIP 2Lm5 2H3m3
EIVLTQSPATLSLSPGERATLSCRASQSVSSYMHWYQQKPGQAPRLLIYAPSNLASGIPARFSGSGSGTDFTLTISSLEPEDFAVYYCQQWSFNPPTFGQGTKVEIKDGGGSGGGGSGGGGTGEVQLLESGGGLVQPGGSLRLSCAASGFTFSSYNMHWVRQAPGKGLEWVSAIYPGNGDTSYNQKFKGRFTISRDNSKNTLYLQMNSLRAEDTAVYYCAKSYYSNSYWYFDLWGRGTLVTVSS

SEQ ID NO: 6 - protein, 244 aa, Artificial Sequence
  SMIP 2L 2Hm
EIVLTQSPATLSLSPGERATLSCRASSSVSSYMHWYQQKPGQAPRLLIYAPSNLASGIPARFSGSGSGTDFTLTISSLEPEDFAVYYCQQWSFNPPTFGQGTKVEIKDGGGSGGGGSGGGGSSQVQLVQSGAEVKKPGASVKVSCKASGYTFTSYNMHWVRQAPGQGLEWMGAIYPGNGDTSYNQKFKGRVTMTRDTSTSTVYMELSSLRSEDTAVYYCARSVYYSNYWYFDLWGRGTLVTVSS

SEQ ID NO: 7 - protein, 243 aa, Artificial Sequence
  SMIP 2Lm 2Hm
EIVLTQSPATLSLSPGERATLSCRASSSVSYMIWYQQKPGQAPRLLIYAISNLASGIPARFSGSGSGTDFTLTISSLEPEDFAVYYCQQWISNPPTFGQGTKVEIKDGGGSGGGGSGGGGSSQVQLVQSGAEVKKPGASVKVSCKASGYTFTSYNMHWVRQAPGQGLEWMGAIYPGNGDTSYNQKFKGRVTMTRDTSTSTVYMELSSLRSEDTAVYYCARSVYYSNYWYFDLWGRGTLVTVSS

SEQ ID NO: 8 - protein, 244 aa, Artificial Sequence
  SMIP 2Lm 2H
EIVLTQSPATLSLSPGERATLSCRASSSVSYMIWYQQKPGQAPRLLIYAISNLASGIPARFSGSGSGTDFTLTISSLEPEDFAVYYCQQWISNPPTFGQGTKVEIKDGGGSGGGGSGGGGSSQVQLVQSGAEVKKPGASVKVSCKASGYTFTSYNMHWVRQAPGQGLEWMGAIYPGNGDTSYNQKFKGRVTMTRDTSTSTVYMELSSLRSEDTAVYYCARVVYYSNSYWYFDLWGRGTLVTVSS

SEQ ID NO: 9 - protein, 244 aa, Artificial Sequence
  SMIP 2Lm1 2Hm
EIVLTQSPATLSLSPGERATLSCRASQSSVSYMHWYQQKPGQAPRLLIYAPSNLASGIPARFSGSGSGTDFTLTISSLEPEDFAVYYCQQWISNPPTFGQGTKVEIKDGGGSGGGGSGGGGSSQVQLVQSGAEVKKPGASVKVSCKASGYTFTSYNMHWVRQAPGQGLEWMGAIYPGNGDTSYNQKFKGRVTMTRDTSTSTVYMELSSLRSEDTAVYYCARSVYYSNYWYFDLWGRGTLVTVSS

SEQ ID NO: 10 - protein, 245 aa, Artificial Sequence
  SMIP 2Lm1 2H
EIVLTQSPATLSLSPGERATLSCRASQSSVSYMHWYQQKPGQAPRLLIYAPSNLASGIPARFSGSGSGTDFTLTISSLEPEDFAVYYCQQWISNPPTFGQGTKVEIKDGGGSGGGGSGGGGSSQVQLVQSGAEVKKPGASVKVSCKASGYTFTSYNMHWVRQAPGQGLEWMGAIYPGNGDTSYNQKFKGRVTMTRDTSTSTVYMELSSLRSEDTAVYYCARVVYYSNSYWYFDLWGRGTLVTVSS

SEQ ID NO: 11 - protein, 243 aa, Artificial Sequence
  SMIP 2Lm2 2Hm
EIVLTQSPATLSLSPGERATLSCRASQSVSYMIWYQQKPGQAPRLLIYAISNLASGIPARFSGSGSGTDFTLTISSLEPEDFAVYYCQQWSFNPPTFGQGTKVEIKDGGGSGGGGSGGGGSSQVQLVQSGAEVKKPGASVKVSCKASGYTFTSYNMHWVRQAPGQGLEWMGAIYPGNGDTSYNQKFKGRVTMTRDTSTSTVYMELSSLRSEDTAVYYCARSVYYSNYWYFDLWGRGTLVTVSS

SEQ ID NO: 12 - protein, 243 aa, Artificial Sequence
  SMIP 2Lm3 2Hm
EIVLTQSPATLSLSPGERATLSCRASSSVSYMIWYQQKPGQAPRLLIYAISNLASGIPARFSGSGSGTDFTLTISSLEPEDFAVYYCQQWTSNPPTFGQGTKVEIKDGGGSGGGGSGGGGSSQVQLVQSGAEVKKPGASVKVSCKASGYTFTSYNMHWVRQAPGQGLEWMGAIYPGNGDTSYNQKFKGRVTMTRDTSTSTVYMELSSLRSEDTAVYYCARSVYYSNYWYFDLWGRGTLVTVSS

SEQ ID NO: 13 - protein, 244 aa, Artificial Sequence
  SMIP 2Lm4 2Hm
EIVLTQSPATLSLSPGERATLSCRASQSVSSYMHWYQQKPGQAPRLLIYAPSNLASGIPARFSGSGSGTDFTLTISSLEPEDFAVYYCQQWTSNPPTFGQGTKVEIKDGGGSGGGGSGGGGSSQVQLVQSGAEVKKPGASVKVSCKASGYTFTSYNMHWVRQAPGQGLEWMGAIYPGNGDTSYNQKFKGRVTMTRDTSTSTVYMELSSLRSEDTAVYYCARSVYYSNYWYFDLWGRGTLVTVSS

SEQ ID NO: 14 - protein, 243 aa, Artificial Sequence
  SMIP 2Lm5 2Hm
EIVLTQSPATLSLSPGERATLSCRASQSVSYMHWYQQKPGQAPRLLIYAPSNLASGIPARFSGSGSGTDFTLTISSLEPEDFAVYYCQQWSFNPPTFGQGTKVEIKDGGGSGGGGSGGGGSSQVQLVQSGAEVKKPGASVKVSCKASGYTFTSYNMHWVRQAPGQGLEWMGAIYPGNGDTSYNQKFKGRVTMTRDTSTSTVYMELSSLRSEDTAVYYCARSVYYSNYWYFDLWGRGTLVTVSS

SEQ ID NO: 15 - protein, 243 aa, Artificial Sequence
  SMIP 2Lm5-1 2Hm3
EIVLTQSPATLSLSPGERATLSCRASQSVSYMHWYQQKPGQAPRLLIYAPSNLASGIPARFSGSGSGTDFTLTISSLEPEDFAVYYCQQWSFNPPTFGQGTKVEIKDGGGSGGGGSGGGGSSQVQLVQSGAEVKKPGASVKVSCKASGYTFTSYNMHWVRQAPGQGLEWMGAIYPGNGDTSYNQKFKGRVTMTRDTSTSTVYMELSSLRSEDTAVYYCARSYYSNSYWYFDLWGRGTLVTVSS

SEQ ID NO: 16 - protein, 243 aa, Artificial Sequence
  SMIP 2Lm5-2 2Hm4
EIVLTQSPATLSLSPGERATLSCRASQSVSYMHWYQQKPGQAPRLLIYAPSNLASGIPARFSGSGSGTDFTLTISSLEPEDFAVYYCQQWSFNPPTFGQGTKVEIKDGGGSGGGGSGGGGSSQVQLVQSGAEVKKPGASVKVSCKASGYTFTSYNMHWVRQAPGQGLEWMGAIYPGNGDTSYNQKFKGRVTMTRDTSTSTVYMELSSLRSEDTAVYYCARVYYSNSYWYFDLWGRGTLVTVSS

SEQ ID NO: 17 - protein, 243 aa, Artificial Sequence
  SMIP 2Lm5-3 2Hm5
EIVLTQSPATLSLSPGERATLSCRASQSVSYMHWYQQKPGQAPRLLIYAPSNLASGIPARFSGSGSGTDFTLTISSLEPEDFAVYYCQQWSFNPPTFGQGTKVEIKDGGGSGGGGSGGGGSSQVQLVQSGAEVKKPGASVKVSCKASGYTFTSYNMHWVRQAPGQGLEWMGAIYPGNGDTSYNQKFKGRVTMTRDTSTSTVYMELSSLRSEDTAVYYCARSVYYNSYWYFDLWGRGTLVTVSS

SEQ ID NO: 18 - protein, 243 aa, Artificial Sequence
  SMIP 2Lm6 2Hm
EIVLTQSPATLSLSPGERATLSCRASQSVSYMHWYQQKPGQAPRLLIYAPSNLASGIPARFSGSGSGTDFTLTISSLEPEDFAVYYCQQWTSNPPTFGQGTKVEIKDGGGSGGGGSGGGGSSQVQLVQSGAEVKKPGASVKVSCKASGYTFTSYNMHWVRQAPGQGLEWMGAIYPGNGDTSYNQKFKGRVTMTRDTSTSTVYMELSSLRSEDTAVYYCARSVYYSNYWYFDLWGRGTLVTVSS

SEQ ID NO: 19 - protein, 243 aa, Artificial Sequence
  SMIP 2Lm6-1 2Hm3
EIVLTQSPATLSLSPGERATLSCRASQSVSYMHWYQQKPGQAPRLLIYAPSNLASGIPARFSGSGSGTDFTLTISSLEPEDFAVYYCQQWTSNPPTFGQGTKVEIKDGGGSGGGGSGGGGSSQVQLVQSGAEVKKPGASVKVSCKASGYTFTSYNMHWVRQAPGQGLEWMGAIYPGNGDTSYNQKFKGRVTMTRDTSTSTVYMELSSLRSEDTAVYYCARSYYSNSYWYFDLWGRGTLVTVSS

SEQ ID NO: 20 - protein, 243 aa, Artificial Sequence
  SMIP 2Lm6-2 2Hm4
EIVLTQSPATLSLSPGERATLSCRASQSVSYMHWYQQKPGQAPRLLIYAPSNLASGIPARFSGSGSGTDFTLTISSLEPEDFAVYYCQQWTSNPPTFGQGTKVEIKDGGGSGGGGSGGGGSSQVQLVQSGAEVKKPGASVKVSCKASGYTFTSYNMHWVRQAPGQGLEWMGAIYPGNGDTSYNQKFKGRVTMTRDTSTSTVYMELSSLRSEDTAVYYCARVYYSNSYWYFDLWGRGTLVTVSS

SEQ ID NO: 21 - protein, 243 aa, Artificial Sequence
  SMIP 2Lm6-3 2Hm5
EIVLTQSPATLSLSPGERATLSCRASQSVSYMHWYQQKPGQAPRLLIYAPSNLASGIPARFSGSGSGTDFTLTISSLEPEDFAVYYCQQWTSNPPTFGQGTKVEIKDGGGSGGGGSGGGGSSQVQLVQSGAEVKKPGASVKVSCKASGYTFTSYNMHWVRQAPGQGLEWMGAIYPGNGDTSYNQKFKGRVTMTRDTSTSTVYMELSSLRSEDTAVYYCARSVYYNSYWYFDLWGRGTLVTVSS

SEQ ID NO: 22 - protein, 243 aa, Artificial Sequence
  SMIP 2Lm7 2Hm
EIVLTQSPATLSLSPGERATLSCRASSSVSYMHWYQQKPGQAPRLLIYATSNLASGIPARFSGSGSGTDFTLTISSLEPEDFAVYYCQQWTSNPPTFGQGTKVEIKDGGGSGGGGSGGGGSSQVQLVQSGAEVKKPGASVKVSCKASGYTFTSYNMHWVRQAPGQGLEWMGAIYPGNGDTSYNQKFKGRVTMTRDTSTSTVYMELSSLRSEDTAVYYCARSVYYSNYWYFDLWGRGTLVTVSS

SEQ ID NO: 23 - protein, 243 aa, Artificial Sequence
  SMIP 2Lm8 2Hm
EIVLTQSPATLSLSPGERATLSCRASSSVSYMIWYQQKPGQAPRLLIYAISNLASGIPARFSGSGSGTDFTLTISSLEPEDFAVYYCQQWISNPYTFGQGTKVEIKDGGGSGGGGSGGGGSSQVQLVQSGAEVKKPGASVKVSCKASGYTFTSYNMHWVRQAPGQGLEWMGAIYPGNGDTSYNQKFKGRVTMTRDTSTSTVYMELSSLRSEDTAVYYCARSVYYSNYWYFDLWGRGTLVTVSS

SEQ ID NO: 24 - protein, 243 aa, Artificial Sequence
  SMIP 2Lm9 2Hm
EIVLTQSPATLSLSPGERATLSCRASSSVSYMIWYQQKPGQAPRLLIYAISNLASGIPARFSGSGSGTDFTLTISSLEPEDFAVYYCQQWISNPFTFGQGTKVEIKDGGGSGGGGSGGGGSSQVQLVQSGAEVKKPGASVKVSCKASGYTFTSYNMHWVRQAPGQGLEWMGAIYPGNGDTSYNQKFKGRVTMTRDTSTSTVYMELSSLRSEDTAVYYCARSVYYSNYWYFDLWGRGTLVTVSS

SEQ ID NO: 25 - protein, 243 aa, Artificial Sequence
  SMIP 2Lm10 2Hm
EIVLTQSPATLSLSPGERATLSCRASSSVSYMIWYQQKPGQAPRLLIYAISNLASGIPARFSGSGSGTDFTLTISSLEPEDFAVYYCQQWISNPLTFGQGTKVEIKDGGGSGGGGSGGGGSSQVQLVQSGAEVKKPGASVKVSCKASGYTFTSYNMHWVRQAPGQGLEWMGAIYPGNGDTSYNQKFKGRVTMTRDTSTSTVYMELSSLRSEDTAVYYCARSVYYSNYWYFDLWGRGTLVTVSS

SEQ ID NO: 26 - protein, 243 aa, Artificial Sequence
  SMIP 2Lm11 2Hm
EIVLTQSPATLSLSPGERATLSCRASSSVSYMIWYQQKPGQAPRLLIYAISNLASGIPARFSGSGSGTDFTLTISSLEPEDFAVYYCQQWISNPITFGQGTKVEIKDGGGSGGGGSGGGGSSQVQLVQSGAEVKKPGASVKVSCKASGYTFTSYNMHWVRQAPGQGLEWMGAIYPGNGDTSYNQKFKGRVTMTRDTSTSTVYMELSSLRSEDTAVYYCARSVYYSNYWYFDLWGRGTLVTVSS

SEQ ID NO: 27 - protein, 243 aa, Artificial Sequence
  SMIP 2Lm12 2Hm
EIVLTQSPATLSLSPGERATLSCRASQSVSYMHWYQQKPGQAPRLLIYATSNLASGIPARFSGSGSGTDFTLTISSLEPEDFAVYYCQQWSFNPPTFGQGTKVEIKDGGGSGGGGSGGGGSSQVQLVQSGAEVKKPGASVKVSCKASGYTFTSYNMHWVRQAPGQGLEWMGAIYPGNGDTSYNQKFKGRVTMTRDTSTSTVYMELSSLRSEDTAVYYCARSVYYSNYWYFDLWGRGTLVTVSS

SEQ ID NO: 28 - protein, 243 aa, Artificial Sequence
  SMIP 2Lm13 2Hm
EIVLTQSPATLSLSPGERATLSCRASQSVSYMHWYQQKPGQAPRLLIYAPSNLASGIPARFSGSGSGTDFTLTISSLEPEDFAVYYCQQWISNPPTFGQGTKVEIKDGGGSGGGGSGGGGSSQVQLVQSGAEVKKPGASVKVSCKASGYTFTSYNMHWVRQAPGQGLEWMGAIYPGNGDTSYNQKFKGRVTMTRDTSTSTVYMELSSLRSEDTAVYYCARSVYYSNYWYFDLWGRGTLVTVSS

SEQ ID NO: 29 - protein, 243 aa, Artificial Sequence
  SMIP 2Lm14 2Hm
EIVLTQSPATLSLSPGERATLSCRASQSVSYMHWYQQKPGQAPRLLIYATSNLASGIPARFSGSGSGTDFTLTISSLEPEDFAVYYCQQWISNPPTFGQGTKVEIKDGGGSGGGGSGGGGSSQVQLVQSGAEVKKPGASVKVSCKASGYTFTSYNMHWVRQAPGQGLEWMGAIYPGNGDTSYNQKFKGRVTMTRDTSTSTVYMELSSLRSEDTAVYYCARSVYYSNYWYFDLWGRGTLVTVSS

SEQ ID NO: 30 - protein, 243 aa, Artificial Sequence
  SMIP 2Lm15 2Hm
EIVLTQSPATLSLSPGERATLSCRASQSVSYIHWYQQKPGQAPRLLIYAPSNLASGIPARFSGSGSGTDFTLTISSLEPEDFAVYYCQQWISNPPTFGQGTKVEIKDGGGSGGGGSGGGGSSQVQLVQSGAEVKKPGASVKVSCKASGYTFTSYNMHWVRQAPGQGLEWMGAIYPGNGDTSYNQKFKGRVTMTRDTSTSTVYMELSSLRSEDTAVYYCARSVYYSNYWYFDLWGRGTLVTVSS

SEQ ID NO: 31 - protein, 243 aa, Artificial Sequence
  SMIP 2Lm16 2Hm3
EIVLTQSPATLSLSPGERATLSCRASSSVSYMHWYQQKPGQAPRLLIYAPSNLASGIPARFSGSGSGTDFTLTISSLEPEDFAVYYCQQWSFNPPTFGQGTKVEIKDGGGSGGGGSGGGGSSQVQLVQSGAEVKKPGASVKVSCKASGYTFTSYNMHWVRQAPGQGLEWMGAIYPGNGDTSYNQKFKGRVTMTRDTSTSTVYMELSSLRSEDTAVYYCARSYYSNSYWYFDLWGRGTLVTVSS

SEQ ID NO: 32 - protein, 242 aa, Artificial Sequence
  SMIP 2Lm17-3 2Hm3
EIVLTQSPATLSLSPGERATLSCRASQSVSYLSWYQQKPGQAPRLLIYAPSNLASGIPARFSGSGSGTDFTLTISSLEPEDFAVYYCQQWSFNPPTFGQGTKVEIKDGGGSGGGGSGGGGSSQVQLVQSGAEVKKPGASVKVSCKASGYTFTSYNMHWVRQAPGQGLEWMGAIYPGNGDTSYNQKFKGRVTMTRDTSTSTVYMELSSLRSEDTAVYYCASYYSNSYWYFDLWGRGTLVTVSS

SEQ ID NO: 33 - protein, 243 aa, Artificial Sequence
  SMIP 2Lm17-4 2Hm3
EIVLTQSPATLSLSPGERATLSCRASQSVSYLTWYQQKPGQAPRLLIYAPSNLASGIPARFSGSGSGTDFTLTISSLEPEDFAVYYCQQWSFNPPTFGQGTKVEIKDGGGSGGGGSGGGGSSQVQLVQSGAEVKKPGASVKVSCKASGYTFTSYNMHWVRQAPGQGLEWMGAIYPGNGDTSYNQKFKGRVTMTRDTSTSTVYMELSSLRSEDTAVYYCARSYYSNSYWYFDLWGRGTLVTVSS

SEQ ID NO: 34 - protein, 243 aa, Artificial Sequence
  SMIP 2Lm17-6 2Hm3
EIVLTQSPATLSLSPGERATLSCRASQSVSYLYWYQQKPGQAPRLLIYAPSNLASGIPARFSGSGSGTDFTLTISSLEPEDFAVYYCQQWSFNPPTFGQGTKVEIKDGGGSGGGGSGGGGSSQVQLVQSGAEVKKPGASVKVSCKASGYTFTSYNMHWVRQAPGQGLEWMGAIYPGNGDTSYNQKFKGRVTMTRDTSTSTVYMELSSLRSEDTAVYYCARSYYSNSYWYFDLWGRGTLVTVSS

SEQ ID NO: 35 - protein, 243 aa, Artificial Sequence
  SMIP 2Lm17-8 2Hm3
EIVLTQSPATLSLSPGERATLSCRASQSVSYLHWYQQKPGQAPRLLIYAPSNLASGIPARFSGSGSGTDFTLTISSLEPEDFAVYYCQQWSFNPPTFGQGTKVEIKDGGGSGGGGSGGGGSSQVQLVQSGAEVKKPGASVKVSCKASGYTFTSYNMHWVRQAPGQGLEWMGAIYPGNGDTSYNQKFKGRVTMTRDTSTSTVYMELSSLRSEDTAVYYCARSYYSNSYWYFDLWGRGTLVTVSS

SEQ ID NO: 36 - protein, 243 aa, Artificial Sequence
  SMIP 2Lm17-12 2Hm3
EIVLTQSPATLSLSPGERATLSCRASQSVSYLNWYQQKPGQAPRLLIYAPSNLASGIPARFSGSGSGTDFTLTISSLEPEDFAVYYCQQWSFNPPTFGQGTKVEIKDGGGSGGGGSGGGGSSQVQLVQSGAEVKKPGASVKVSCKASGYTFTSYNMHWVRQAPGQGLEWMGAIYPGNGDTSYNQKFKGRVTMTRDTSTSTVYMELSSLRSEDTAVYYCARSYYSNSYWYFDLWGRGTLVTVSS

SEQ ID NO: 37 - protein, 243 aa, Artificial Sequence
  SMIP 2L17-14 2Hm3
EIVLTQSPATLSLSPGERATLSCRASQSVSYLAWYQQKPGQAPRLLIYAPSNLASGIPARFSGSGSGTDFTLTISSLEPEDFAVYYCQQWSFNPPTFGQGTKVEIKDGGGSGGGGSGGGGSSQVQLVQSGAEVKKPGASVKVSCKASGYTFTSYNMHWVRQAPGQGLEWMGAIYPGNGDTSYNQKFKGRVTMTRDTSTSTVYMELSSLRSEDTAVYYCARSYYSNSYWYFDLWGRGTLVTVSS

SEQ ID NO: 38 - protein, 243 aa, Artificial Sequence
  SMIP 2Lm18-2 2Hm3
EIVLTQSPATLSLSPGERATLSCRASSSVSYLAWYQQKPGQAPRLLIYAPSNLASGIPARFSGSGSGTDFTLTISSLEPEDFAVYYCQQWSFNPPTFGQGTKVEIKDGGGSGGGGSGGGGSSQVQLVQSGAEVKKPGASVKVSCKASGYTFTSYNMHWVRQAPGQGLEWMGAIYPGNGDTSYNQKFKGRVTMTRDTSTSTVYMELSSLRSEDTAVYYCARSYYSNSYWYFDLWGRGTLVTVSS

SEQ ID NO: 39 - protein, 243 aa, Artificial Sequence
  SMIP 2Lm18-3 2Hm3
EIVLTQSPATLSLSPGERATLSCRASSSVSYLNWYQQKPGQAPRLLIYAPSNLASGIPARFSGSGSGTDFTLTISSLEPEDFAVYYCQQWSFNPPTFGQGTKVEIKDGGGSGGGGSGGGGSSQVQLVQSGAEVKKPGASVKVSCKASGYTFTSYNMHWVRQAPGQGLEWMGAIYPGNGDTSYNQKFKGRVTMTRDTSTSTVYMELSSLRSEDTAVYYCARSYYSNSYWYFDLWGRGTLVTVSS

SEQ ID NO: 40 - protein, 243 aa, Artificial Sequence
  SMIP 2Lm18-4 2Hm3
EIVLTQSPATLSLSPGERATLSCRASSSVSYLDWYQQKPGQAPRLLIYAPSNLASGIPARFSGSGSGTDFTLTISSLEPEDFAVYYCQQWSFNPPTFGQGTKVEIKDGGGSGGGGSGGGGSSQVQLVQSGAEVKKPGASVKVSCKASGYTFTSYNMHWVRQAPGQGLEWMGAIYPGNGDTSYNQKFKGRVTMTRDTSTSTVYMELSSLRSEDTAVYYCARSYYSNSYWYFDLWGRGTLVTVSS

SEQ ID NO: 41 - protein, 243 aa, Artificial Sequence
  SMIP 2Lm18-5 2Hm3
EIVLTQSPATLSLSPGERATLSCRASSSVSYLSWYQQKPGQAPRLLIYAPSNLASGIPARFSGSGSGTDFTLTISSLEPEDFAVYYCQQWSFNPPTFGQGTKVEIKDGGGSGGGGSGGGGSSQVQLVQSGAEVKKPGASVKVSCKASGYTFTSYNMHWVRQAPGQGLEWMGAIYPGNGDTSYNQKFKGRVTMTRDTSTSTVYMELSSLRSEDTAVYYCARSYYSNSYWYFDLWGRGTLVTVSS

SEQ ID NO: 42 - protein, 243 aa, Artificial Sequence
  SMIP 2L18-4 2Hm3
EIVLTQSPATLSLSPGERATLSCRASSSVSYLHWYQQKPGQAPRLLIYAPSNLASGIPARFSGSGSGTDFTLTISSLEPEDFAVYYCQQWSFNPPTFGQGTKVEIKDGGGSGGGGSGGGGSSQVQLVQSGAEVKKPGASVKVSCKASGYTFTSYNMHWVRQAPGQGLEWMGAIYPGNGDTSYNQKFKGRVTMTRDTSTSTVYMELSSLRSEDTAVYYCARSYYSNSYWYFDLWGRGTLVTVSS

SEQ ID NO: 43 - protein, 243 aa, Artificial Sequence
  SMIP 2Lm19-1 2Hm3
EIVLTQSPATLSLSPGERATLSCRASQSVSYIDWYQQKPGQAPRLLIYAPSNLASGIPARFSGSGSGTDFTLTISSLEPEDFAVYYCQQWSFNPPTFGQGTKVEIKDGGGSGGGGSGGGGSSQVQLVQSGAEVKKPGASVKVSCKASGYTFTSYNMHWVRQAPGQGLEWMGAIYPGNGDTSYNQKFKGRVTMTRDTSTSTVYMELSSLRSEDTAVYYCARSYYSNSYWYFDLWGRGTLVTVSS

SEQ ID NO: 44 - protein, 243 aa, Artificial Sequence
  SMIP 2Lm19-2 2Hm3
EIVLTQSPATLSLSPGERATLSCRASQSVSYISWYQQKPGQAPRLLIYAPSNLASGIPARFSGSGSGTDFTLTISSLEPEDFAVYYCQQWSFNPPTFGQGTKVEIKDGGGSGGGGSGGGGSSQVQLVQSGAEVKKPGASVKVSCKASGYTFTSYNMHWVRQAPGQGLEWMGAIYPGNGDTSYNQKFKGRVTMTRDTSTSTVYMELSSLRSEDTAVYYCARSYYSNSYWYFDLWGRGTLVTVSS

SEQ ID NO: 45 - protein, 243 aa, Artificial Sequence
  SMIP 2Lm19-3 2Hm3
EIVLTQSPATLSLSPGERATLSCRASQSVSYIVWYQQKPGQAPRLLIYAPSNLASGIPARFSGSGSGTDFTLTISSLEPEDFAVYYCQQWSFNPPTFGQGTKVEIKDGGGSGGGGSGGGGSSQVQLVQSGAEVKKPGASVKVSCKASGYTFTSYNMHWVRQAPGQGLEWMGAIYPGNGDTSYNQKFKGRVTMTRDTSTSTVYMELSSLRSEDTAVYYCARSYYSNSYWYFDLWGRGTLVTVSS

SEQ ID NO: 46 - protein, 243 aa, Artificial Sequence
  SMIP 2Lm19-4 2Hm3
EIVLTQSPATLSLSPGERATLSCRASQSVSYIAWYQQKPGQAPRLLIYAPSNLASGIPARFSGSGSGTDFTLTISSLEPEDFAVYYCQQWSFNPPTFGQGTKVEIKDGGGSGGGGSGGGGSSQVQLVQSGAEVKKPGASVKVSCKASGYTFTSYNMHWVRQAPGQGLEWMGAIYPGNGDTSYNQKFKGRVTMTRDTSTSTVYMELSSLRSEDTAVYYCARSYYSNSYWYFDLWGRGTLVTVSS

SEQ ID NO: 47 - protein, 243 aa, Artificial Sequence
  SMIP 2Lm19-7 2Hm3
EIVLTQSPATLSLSPGERATLSCRASQSVSYITWYQQKPGQAPRLLIYAPSNLASGIPARFSGSGSGTDFTLTISSLEPEDFAVYYCQQWSFNPPTFGQGTKVEIKDGGGSGGGGSGGGGSSQVQLVQSGAEVKKPGASVKVSCKASGYTFTSYNMHWVRQAPGQGLEWMGAIYPGNGDTSYNQKFKGRVTMTRDTSTSTVYMELSSLRSEDTAVYYCARSYYSNSYWYFDLWGRGTLVTVSS

SEQ ID NO: 48 - protein, 220 aa, Artificial Sequence
  SMIP 2Lm19-9 2Hm3
EIVLTQSPATLSLSPGERATLSCRASQSVSYIIWYQQKPGQAPRLLIYAPSNLASGIPARFSGSGSGTDFTLTISSLEPEDFAVYYCQQWSFNPPTFGQGTKVEIKDGGGSGGGGSGGGGSSQVQLVQSGAEVKKPGASVKVSCKASGYTFTSYNMHWVRQAPGQGLEWMGAIYPGNGDTSYNQKFKGRVTMTRDTSTSTVYMELSSLRSEDTAVYYCAR

SEQ ID NO: 49 - protein, 243 aa, Artificial Sequence
  SMIP 2Lm19-12 2Hm3
EIVLTQSPATLSLSPGERATLSCRASQSVSYIPWYQQKPGQAPRLLIYAPSNLASGIPARFSGSGSGTDFTLTISSLEPEDFAVYYCQQWSFNPPTFGQGTKVEIKDGGGSGGGGSGGGGSSQVQLVQSGAEVKKPGASVKVSCKASGYTFTSYNMHWVRQAPGQGLEWMGAIYPGNGDTSYNQKFKGRVTMTRDTSTSTVYMELSSLRSEDTAVYYCARSYYSNSYWYFDLWGRGTLVTVSS

SEQ ID NO: 50 - protein, 243 aa, Artificial Sequence
  SMIP 2Lm19-14 2Hm3
EIVLTQSPATLSLSPGERATLSCRASQSVSYINWYQQKPGQAPRLLIYAPSNLASGIPARFSGSGSGTDFTLTISSLEPEDFAVYYCQQWSFNPPTFGQGTKVEIKDGGGSGGGGSGGGGSSQVQLVQSGAEVKKPGASVKVSCKASGYTFTSYNMHWVRQAPGQGLEWMGAIYPGNGDTSYNQKFKGRVTMTRDTSTSTVYMELSSLRSEDTAVYYCARSYYSNSYWYFDLWGRGTLVTVSS

SEQ ID NO: 51 - protein, 243 aa, Artificial Sequence
  SMIP 2Lm20-1 2Hm3
EIVLTQSPATLSLSPGERATLSCRASSSVSYISWYQQKPGQAPRLLIYAPSNLASGIPARFSGSGSGTDFTLTISSLEPEDFAVYYCQQWSFNPPTFGQGTKVEIKDGGGSGGGGSGGGGSSQVQLVQSGAEVKKPGASVKVSCKASGYTFTSYNMHWVRQAPGQGLEWMGAIYPGNGDTSYNQKFKGRVTMTRDTSTSTVYMELSSLRSEDTAVYYCARSYYSNSYWYFDLWGRGTLVTVSS

SEQ ID NO: 52 - protein, 243 aa, Artificial Sequence
  SMIP 2Lm20-2 2Hm3
EIVLTQSPATLSLSPGERATLSCRASSSVSYIAWYQQKPGQAPRLLIYAPSNLASGIPARFSGSGSGTDFTLTISSLEPEDFAVYYCQQWSFNPPTFGQGTKVEIKDGGGSGGGGSGGGGSSQVQLVQSGAEVKKPGASVKVSCKASGYTFTSYNMHWVRQAPGQGLEWMGAIYPGNGDTSYNQKFKGRVTMTRDTSTSTVYMELSSLRSEDTAVYYCARSYYSNSYWYFDLWGRGTLVTVSS

SEQ ID NO: 53 - protein, 243 aa, Artificial Sequence
  SMIP 2Lm20-4 2Hm3
EIVLTQSPATLSLSPGERATLSCRASSSVSYIVWYQQKPGQAPRLLIYAPSNLASGIPARFSGSGSGTDFTLTISSLEPEDFAVYYCQQWSFNPPTFGQGTKVEIKDGGGSGGGGSGGGGSSQVQLVQSGAEVKKPGASVKVSCKASGYTFTSYNMHWVRQAPGQGLEWMGAIYPGNGDTSYNQKFKGRVTMTRDTSTSTVYMELSSLRSEDTAVYYCARSYYSNSYWYFDLWGRGTLVTVSS

SEQ ID NO: 54 - protein, 243 aa, Artificial Sequence
  SMIP 2Lm20-8 2Hm3
EIVLTQSPATLSLSPGERATLSCRASSSVNYIYWYQQKPGQAPRLLIYAPSNLASGIPARFSGSGSGTDFTLTISSLEPEDFAVYYCQQWSFNPPTFGQGTKVEIKDGGGSGGGGSGGGGSSQVQLVQSGAEVKKPGASVKVSCKASGYTFTSYNMHWVRQAPGQGLEWMGAIYPGNGDTSYNQKFKGRVTMTRDTSTSTVYMELSSLRSEDTAVYYCARSYYSNSYWYFDLWGRGTLVTVSS

SEQ ID NO: 55 - protein, 243 aa, Artificial Sequence
  SMIP 2Lm20-11 2Hm3
EIVLTQSPATLSLSPGERATLSCRASSSVSYIDWYQQKPGQAPRLLIYAPSNLASGIPARFSGSGSGTDFTLTISSLEPEDFAVYYCQQWSFNPPTFGQGTKVEIKDGGGSGGGGSGGGGSSQVQLVQSGAEVKKPGASVKVSCKASGYTFTSYNMHWVRQAPGQGLEWMGAIYPGNGDTSYNQKFKGRVTMTRDTSTSTVYMELSSLRSEDTAVYYCARSYYSNSYWYFDLWGRGTLVTVSS

SEQ ID NO: 56 - protein, 243 aa, Artificial Sequence
  SMIP 2Lm20-12 2Hm3
EIVLTQSPATLSLSPGERATLSCRASSSVSYIIWYQQKPGQAPRLLIYAPSNLASGIPARFSGSGSGTDFTLTISSLEPEDFAVYYCQQWSFNPPTFGQGTKVEIKDGGGSGGGGSGGGGSSQVQLVQSGAEVKKPGASVKVSCKASGYTFTSYNMHWVRQAPGQGLEWMGAIYPGNGDTSYNQKFKGRVTMTRDTSTSTVYMELSSLRSEDTAVYYCARSYYSNSYWYFDLWGRGTLVTVSS

SEQ ID NO: 57 - protein, 243 aa, Artificial Sequence
  SMIP 2Lm20-13 2Hm3
EIVLTQSPATLSLSPGERATLSCRASSSVSYIYWYQQKPGQAPRLLIYAPSNLASGIPARFSGSGSGTDFTLTISSLEPEDFAVYYCQQWSFNPPTFGQGTKVEIKDGGGSGGGGSGGGGSSQVQLVQSGAEVKKPGASVKVSCKASGYTFTSYNMHWVRQAPGQGLEWMGAIYPGNGDTSYNQKFKGRVTMTRDTSTSTVYMELSSLRSEDTAVYYCARSYYSNSYWYFDLWGRGTLVTVSS

SEQ ID NO: 58 - protein, 244 aa, Artificial Sequence; identical to SEQ ID NO: 2
  SMIP 2Lm5(18009) 2H5m3

SEQ ID NO: 59 - protein, 243 aa, Artificial Sequence
  2Lm5 (2Lm5-2H3m3) 2H3m3
EIVLTQSPATLSLSPGERATLSCRASQSVSYMHWYQQKPGQAPRLLIYAPSNLASGIPARFSGSGSGTDFTLTISSLEPEDFAVYYCQQWSFNPPTFGQGTKVEIKDGGGSGGGGSGGGGTGEVQLLESGGGLVQPGGSLRLSCAASGFTFSSYNMHWVRQAPGKGLEWVSAIYPGNGDTSYNQKFKGRFTISRDNSKNTLYLQMNSLRAEDTAVYYCAKSYYSNSYWYFDLWGRGTLVTVSS

SEQ ID NO: 60 - protein, 17 aa, Artificial Sequence
  Hinge CSSS
DQEPKSCDKTHTSPPSS

SEQ ID NO: 61 - protein, 17 aa, Artificial Sequence
  Hinge WT
DQEPKSCDKTHTCPPCP

SEQ ID NO: 62 - protein, 17 aa, Artificial Sequence
  Hinge CSCS
DQEPKSCDKTHTSPPCS

SEQ ID NO: 63 - protein, 17 aa, Artificial Sequence
  Hinge SCCS
DQEPKSSDKTHTCPPCS

SEQ ID NO: 64 - protein, 17 aa, Artificial Sequence
  Hinge SCCP
DQEPKSSDKTHTCPPCP

SEQ ID NO: 65 - protein, 217 aa, Artificial Sequence
  Ch2CH3 WT
APELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 66 - protein, 217 aa, Artificial Sequence
  CH2CH3 P331S
APELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPASIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 67 - protein, 477 aa, Artificial Sequence
  Exemplary Full Length SMIP
EIVLTQSPATLSLSPGERATLSCRASQSVSYIVWYQQKPGQAPRLLIYAPSNLASGIPARFSGSGSGTDFTLTISSLEPEDFAVYYCQQWSFNPPTFGQGTKVEIKDGGGSGGGGSGGGGTGEVQLVQSGAEVKKPGESLKISCKGSGYSFTSYNMHWVRQMPGKGLEWMGAIYPGNGDTSYNQKFKGQVTISADKSISTAYLQWSSLKASDTAMYYCARSYYSNSYWYFDLWGRGTLVTVSSDQEPKSSDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPASIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 68 - protein, 477 aa, Artificial Sequence
  Exemplary Full Length SMIP
EIVLTQSPATLSLSPGERATLSCRASSSVSYIVWYQQKPGQAPRLLIYAPSNLASGIPARFSGSGSGTDFTLTISSLEPEDFAVYYCQQWSFNPPTFGQGTKVEIKDGGGSGGGGSGGGGSSQVQLVQSGAEVKKPGASVKVSCKASGYTFTSYNMHWVRQAPGQGLEWMGAIYPGNGDTSYNQKFKGRVTMTRDTSTSTVYMELSSLRSEDTAVYYCARSYYSNSYWYFDLWGRGTLVTVSSDQEPKSSDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPASIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 69 - protein, 477 aa, Artificial Sequence
  Exemplary Full Length SMIP
QIVLSQSPAILSASPGEKVTMTCRASSSVSYMHWYQQKPGSSPKPWIYAPSNLASGVPARFSGSGSGTSYSLTISRVEAEDAATYYCQQWSFNPPTFGAGTKLELKDGGGSGGGGSGGGGSSQAYLQQSGAESVRPGASVKMSCKASGYTFTSYNMHWVKQTPRQGLEWIGAIYPGNGDTSYNQKFKGKATLTVDKSSSTAYMQLSSLTSEDSAVYFCARVVYYSNSYWYFDVWGTGTTVTVSDQEPKSCDKTHTSPPCSAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 70 - protein, 477 aa, Artificial Sequence
  Exemplary Full Length SMIP
EIVLTQSPATLSLSPGERATLSCRASQSVSYIVWYQQKPGQAPRLLIYAPSNLASGIPARFSGSGSGTDFTLTISSLEPEDFAVYYCQQWSFNPPTFGQGTKVEIKDGGGSGGGGSGGGGTGEVQLVQSGAEVKKPGESLKISCKGSGYSFTSYNMHWVRQMPGKGLEWMGAIYPGNGDTSYNQKFKGQVTISADKSISTAYLQWSSLKASDTAMYYCARSYYSNSYWYFDLWGRGTLVTVSSDQEPKSSDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 71 - protein, 477 aa, Artificial Sequence
  Exemplary Full Length SMIP
EIVLTQSPATLSLSPGERATLSCRASSSVSYIVWYQQKPGQAPRLLIYAPSNLASGIPARFSGSGSGTDFTLTISSLEPEDFAVYYCQQWSFNPPTFGQGTKVEIKDGGGSGGGGSGGGGSSQVQLVQSGAEVKKPGASVKVSCKASGYTFTSYNMHWVRQAPGQGLEWMGAIYPGNGDTSYNQKFKGRVTMTRDTSTSTVYMELSSLRSEDTAVYYCARSYYSNSYWYFDLWGRGTLVTVSSDQEPKSSDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 72 - protein, 477 aa, Artificial Sequence
  Exemplary Full Length SMIP
EIVLTQSPATLSLSPGERATLSCRASSSVSYIDWYQQKPGQAPRLLIYAPSNLASGIPARFSGSGSGTDFTLTISSLEPEDFAVYYCQQWSFNPPTFGQGTKVEIKDGGGSGGGGSGGGGSSQVQLVQSGAEVKKPGASVKVSCKASGYTFTSYNMHWVRQAPGQGLEWMGAIYPGNGDTSYNQKFKGRVTMTRDTSTSTVYMELSSLRSEDTAVYYCARSYYSNSYWYFDLWGRGTLVTVSSDQEPKSCDKTHTSPPSSAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 73 - protein, 477 aa, Artificial Sequence; identical to SEQ ID NO: 71
  Exemplary Full Length SMIP

SEQ ID NO: 74 - protein, 478 aa, Artificial Sequence
  Exemplary Full Length SMIP
EIVLTQSPATLSLSPGERATLSCRASQSVSYIVWYQQKPGQAPRLLIYAPSNLASGIPARFSGSGSGTDFTLTISSLEPEDFAVYYCQQWSFNPPTFGQGTKVEIKDGGGSGGGGSGGGGTGEVQLVQSGAEVKKPGESLKISCKGSGYSFTSYNMHWVRQMPGKGLEWMGAIYPGNGDTSYNQKFKGQVTISADKSISTAYLQWSSLKASDTAMYYCARVVYYSNSYWYFDLWGRGTLVTVSSDQEPKSCDKTHTSPPCSAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPASIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 75 - protein, 477 aa, Artificial Sequence
  Exemplary Full Length SMIP
EIVLTQSPATLSLSPGERATLSCRASSSVSYMIWYQQKPGQAPRLLIYAISNLASGIPARFSGSGSGTDFTLTISSLEPEDFAVYYCQQWISNPLTFGQGTKVEIKDGGGSGGGGSGGGGSSQVQLVQSGAEVKKPGASVKVSCKASGYTFTSYNMHWVRQAPGQGLEWMGAIYPGNGDTSYNQKFKGRVTMTRDTSTSTVYMELSSLRSEDTAVYYCARSVYYSNYWYFDLWGRGTLVTVSSDQEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 76 - protein, 477 aa, Artificial Sequence
  Exemplary Full Length SMIP
EIVLTQSPATLSLSPGERATLSCRASSSVSYIIWYQQKPGQAPRLLIYAPSNLASGIPARFSGSGSGTDFTLTISSLEPEDFAVYYCQQWSFNPPTFGQGTKVEIKDGGGSGGGGSGGGGSSQVQLVQSGAEVKKPGASVKVSCKASGYTFTSYNMHWVRQAPGQGLEWMGAIYPGNGDTSYNQKFKGRVTMTRDTSTSTVYMELSSLRSEDTAVYYCARSYYSNSYWYFDLWGRGTLVTVSSDQEPKSCDKTHTSPPSSAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPASIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPGK